(19) United States
(12) Patent Application Publication
(10) Pub. No.: US 2015/0152395 A1
(43) Pub. Date: Jun. 4, 2015

(54) CHIMERIC LUCIFERASES

(71) Applicant: Connecticut College, New London, CT (US)

(72) Inventor: Bruce Branchini, New London, CT (US)

(73) Assignee: CONNECTICUT COLLEGE, New London, CT (US)

(21) Appl. No.: 14/551,377

(22) Filed: Nov. 24, 2014

Related U.S. Application Data
(63) Continuation of application No. PCT/US2013/042178, filed on May 22, 2013.
(60) Provisional application No. 61/753,606, filed on Jan. 17, 2013.
(60) Provisional application No. 61/651,497, filed on May 24, 2012.

(51) Int. Cl. C12N 9/02; C12Q 1/66

(52) U.S. Cl. CPC C12N 9/0069; C12Q 1/66; C07K 2319/61; C12Y 113/12007

(57) ABSTRACT

Described herein are novel chimeric luciferase molecules with enhanced properties, and methods of using these chimeric luciferase molecules.

### CROSS-REFERENCE TO RELATED APPLICATIONS

This application claims priority to U.S. Provisional Application Ser. No. 61/651,497 filed May 24, 2012, and U.S. Provisional Application Ser. No. 61/753,606 filed Jan. 17, 2013, the entire contents of which is incorporated herein.

### SEQUENCE LISTING

The instant application contains a Sequence Listing which has been submitted in ASCII format via EFS-Web and is hereby incorporated by reference in its entirety. Said ASCII copy, created on Jun. 19, 2013, is named 121499-00120_SL.txt and is 208,104 bytes in size.

### BACKGROUND

The use of reporter molecules or labels to qualitatively or quantitatively molecular events is well established. They are found in assays for medical diagnosis, for the detection of toxins and other substances in industrial environments, and for basic and applied research in biology, biomedicine, and biochemistry. Such assays include immunoassays, nucleic acid probe hybridization assays, and assays in which a reporter enzyme or other protein is produced by expression under control of a particular promoter. Reporter molecules, or labels in such assay systems, have included radioactive isotopes, fluorescent agents, enzymes and chemiluminescent agents.

Light-emitting systems have been known and isolated from many luminescent organisms including bacteria, protozoa, coelenterates, molluscs, fish, millipedes, flies, fungi, worms, crustaceans, and beetles, particularly click beetles of genus Pyrophorus and the fireflies of the genera Photinus, Photuris, and Luciola. In many of these organisms, enzymes catalyze monooxygenations and utilize the resulting free energy to excite a molecule to a high energy state. Visible light is emitted when the excited molecule spontaneously returns to the ground state. This emitted light is called “bioluminescence” or “luminescence.” The North American firefly Photinus pyralis, known for its flash of yellow-green light, houses one of the most efficient bioluminescent systems ever studied.

Luciferase genes are widely used as genetic reporters due to the non-radioactive nature, sensitivity, and linear range of luminescence assays. Consequently, luciferase assays of gene activity are used in virtually every experimental biological system, including both prokaryotic and eukaryotic cell cultures, transgenic plants and animals, and cell-free expression systems. Similarly, luciferase assays of ATP are highly sensitive.

Luciferases generate light via the oxidation of enzyme-specific substrates, called luciferins. For firefly luciferase and all other beetle luciferases, this is done in the presence of magnesium ions, oxygen, and ATP. For anthozoan luciferases, including Renilla luciferase, only oxygen is required along with the luciferin. Generally, in luminescence assays of genetic activity, reaction substrates and other luminescence-activating reagents are introduced into a biological system suspected of expressing a reporter enzyme. Resultant luminescence, if any, is then measured using a luminometer or any suitable radiant energy-measuring device. The assay is very rapid and sensitive, and provides gene expression data quickly and easily, without the need for radioactive reagents. Reporter assays other than for genetic activity are performed analogously.

Achieving greater light intensity from the assay reagent is made difficult because of an inherent trade-off with the stability of the luminescent signal. While chimeric proteins containing various luciferases joined to non-bioluminescent proteins have been made and used in a variety of bioanalytical applications, there have been few reports of chimeric luciferases produced from regions of beetle luciferase sequences. Moreover, in such studies, there was no data presented to indicate a greater overall production of light representative of an enzyme that is more catalytically active than wild-type Luciferase.

Accordingly, a luciferase enzyme that offers enhanced emission sensitivity and stability, qualities that are challenging to achieve simultaneously, would be especially useful in dual-color reporter assays or in in vivo imaging and reporter gene assays.

### SUMMARY OF THE INVENTION

The present invention is based on the unexpected finding that novel chimeric P. pyralis luciferases demonstrate unusually enhanced properties compared to recombinant P. pyralis luciferase, as well as native luciferase (Luc) isolated from firefly lanterns as well as Luciola italica luciferase.

Accordingly, the present invention provides in a first aspect a chimeric firefly luciferase comprising an N-terminal amino acid domain from a first firefly luciferase and a C-terminal amino acid domain from a second firefly luciferase.

In another aspect, the invention features a firefly luciferase comprising an N-terminal amino acid domain from a first firefly luciferase and a C-terminal amino acid domain from a second firefly luciferase, wherein the N-terminal amino acid domain is from Photinius pyralis (P. pyralis; Ppy) luciferase and the C-terminal amino acid domain is from Luciola italica (L. italica; Lit) luciferase.

In other embodiments, the firefly luciferase further comprises a linker peptide. In related embodiments, the linker peptide is a tripeptide linker. In other related embodiments, the linker peptide comprises ArgLeuLys or ArgTyrLys. In still further embodiments, the linker peptide further comprises a mutation. In other exemplary embodiments, the linker peptide comprises residues 437-439 of SEQ ID NO:4.

In preferred embodiments, the L italica luciferase comprises the amino acid sequence of SEQ ID NO:2 or SEQ ID NO:6. In further preferred embodiments, SEQ ID NO:2 is encoded by the nucleic acid sequence of SEQ ID NO: 1. In other further preferred embodiments, SEQ ID NO:6 is encoded by the nucleic acid sequence of SEQ ID NO: 5.

In preferred embodiments, the C-terminal amino acid domain comprises the amino acid sequence of SEQ ID NO:2 or SEQ ID NO:6, and the N-terminal amino acid domain is from a second firefly luciferase. In further preferred embodiments, the N-terminal amino acid domain is from P. pyralis luciferase.

In preferred embodiments, the P. pyralis luciferase comprises the amino acid sequence of SEQ ID NO:4. In further preferred embodiments, SEQ ID NO:4 is encoded by the nucleic acid sequence of SEQ ID NO:3.

In preferred embodiments, the N-terminal amino acid domain comprises the amino acid sequence of SEQ ID NO:4 and the C-terminal amino acid domain is from a second firefly luciferase. In related preferred embodiments, the C-terminal amino acid domain is from L. italica luciferase.

In other preferred embodiments, the P. pyralis luciferase comprises an N-terminal extension peptide. In related preferred embodiments, the N-terminal extension peptide is selected from the group consisting of: GPLGS (SEQ ID NO: 25) and HisTag.

In still other preferred embodiments, the P. pyralis luciferase comprises an N-terminal extension peptide. In related preferred embodiments, the N-terminal extension peptide is selected from the group consisting of: GPLGS (SEQ ID NO: 25) and HisTag.

In preferred embodiments, the N-terminal domain comprises residues 1-436 of SEQ ID NO: 4, and the C-terminal domain comprises residues 442-548 of SEQ ID NO:6.

In other preferred embodiments, the firefly luciferase comprises the nucleic acid sequence of SEQ ID NO:7. In still other preferred embodiments, the firefly luciferase comprises the amino acid sequence of SEQ ID NO:8.

The present invention also features a firefly luciferase comprising the amino acid sequence of SEQ ID NO:4.

In preferred embodiments, the firefly luciferase further comprises one or more amino acid changes selected from the group consisting of: A450P, I457V, L472V, D475S, D476E, A482G, L487M, H489K, A503N, T507V, T508N, A509H, K511R and V517R.

In other preferred embodiments, the firefly luciferase further comprises one or more amino acid changes selected from the group consisting of: A450P, I457V, L472V, D475S, D476E, A482G, L487M, H489K, A503N, T507V, T508N, A509H, K511R, V517R, L530I, K534V, 1540K, A542P and K543Q.

In another exemplary embodiment, the firefly luciferase comprises one or more amino acid changes selected from the group consisting of I457V, A482G, H489K, A503N and K543Q. In a related exemplary embodiment, the firefly luciferase comprises the amino acid changes I457V, A482G, H489K, A503N and K543Q. In further related embodiments, the firefly luciferase comprises the amino acid sequence of SEQ ID NO 10. In another related embodiment, SEQ ID NO:10 is encoded by the nucleic acid sequence of SEQ ID NO:9.

In still other preferred embodiments, the firefly luciferase further comprises the amino acid change F465R.

In preferred embodiments, the firefly luciferase further comprises an amino acid change I232A/E354K. In other preferred embodiments, the firefly luciferase further comprises an amino acid change I351V/E354K. In other preferred embodiments, the firefly luciferase further comprises an amino acid change I232A/I351V/E354K. In other preferred embodiments, the firefly luciferase further comprises an amino acid change I351V/E354K/F465R. In other preferred embodiments, the firefly luciferase further comprises the amino acid change S284T. In other preferred embodiments, the firefly luciferase further comprises the amino acid change S284T/F465R. In other preferred embodiments, the firefly luciferase further comprises the amino acid change S284T/I351V/E354K. In other preferred embodiments, the firefly luciferase further comprises the amino acid change I232A/S284T/I351V/E354I. In other preferred embodiments, the firefly luciferase further comprises the amino acid change S284T/I351V/E354K/F465R. In other preferred embodiments, the firefly luciferase further comprises the amino acid change I232A/S284T/I351V/E354K/F465R.

In further preferred embodiments, the firefly luciferase further comprises one or more amino acid changes selected from the group consisting of: F465R, I232A, E354K, I351V, I232A, S284T, E354I, T214A, A215L, and F295L.

In preferred embodiments, the firefly luciferase further comprises the amino acid change T214A/A215L/I232A/V241I/G246A/F250S/F295L/E354K.

In other preferred embodiments, the firefly luciferase further comprises the amino acid change T214A/A215L/I232A/S284T/F295L/E354K.

In further preferred embodiments, the firefly luciferase further comprises one or more amino acid changes selected from the group consisting of: T214A, A215L, I232A, V241I, G246A, F250S, F295L, E354K, S284T and I351V.

In other aspects, the invention features a firefly luciferase comprising the amino acid sequence of SEQ ID NO: 22, with one or more amino acid changes selected from the group consisting of Ile457Val, Ala482Gly, His489Lys, Ala503Asn, Lys543Gln and Ile351Val. In one embodiment, the firefly luciferase comprising the amino acid sequence of SEQ ID NO:22, comprises the amino acid change Ile457Val/Ala482Gly/His489Lys/Ala503Asn/Lys543Gln/Ile351Val. In a related embodiment, the firefly luciferase comprises the amino acid sequence of SEQ ID NO 12. In another related embodiment, SEQ ID NO:12 is encoded by the nucleic acid sequence of SEQ ID NO:11.

In another aspect, the invention features a firefly luciferase comprising the amino acid sequence of SEQ ID NO: 24, with one or more amino acid changes selected from the group consisting of Ile457Val, Ala482Gly, His489Lys, Ala503Asn, Lys543Gln and Ile351Val. In one embodiment, the firefly luciferase comprising the amino acid sequence of SEQ ID NO:24, comprises the amino acid change Ile457Val/Ala482Gly/His489Lys/Ala503Asn/Lys543Gln/Ile351Val. In a related embodiment, the firefly luciferase comprises the amino acid sequence of SEQ ID NO 14. In another related embodiment, SEQ ID NO:14 is encoded by the nucleic acid sequence of SEQ ID NO:13.

In another aspect, the invention features a firefly luciferase comprising the amino acid sequence of SEQ ID NO: 20, with one or more amino acid changes selected from the group consisting of Ile457Val, Arg465Phe, Ala482Gly, His489Lys, Ala503Asn and Lys543Gln. In one embodiment, the firefly luciferase comprising the amino acid sequence of SEQ ID NO:20, comprises the amino acid change Ile457Val/Arg465Phe/Ala482Gly/His489Lys/Ala503Asn/Lys543Gln. In a related embodiment, the firefly luciferase comprises the amino acid sequence of SEQ ID NO 16. In another related embodiment, SEQ ID NO:16 is encoded by the nucleic acid sequence of SEQ ID NO:15. In a further embodiment, the sequence is codon optimized.

In another aspect, the invention features a codon optimized firefly lucerifase comprising the amino acid sequence of SEQ ID NO:18. In a related embodiment, SEQ ID NO:18 is encoded by the nucleic acid sequence of SEQ ID NO:17.

In certain embodiments of any one of the acove aspects, the thermostability of the luciferase is increased compared to the P. pyralis luciferase.

In further preferred embodiments of the present invention, the resistance to color shifting of the firefly luciferase is increased compared to the P. pyralis luciferase.

In other embodiments of the present invention, the flash-height activity, integration specific activity or catalytic efficiency of the luciferase is increased.

In other further embodiments of the present invention, the luciferase is resistant to red shifting of light emission at low pH.

In still other embodiments of the present invention, the firefly luciferase has the ability to emit red light at a wavelength of about 607 to 614 nm.

In another preferred embodiment of the present invention, the firefly luciferase further comprises an N-terminal peptide extension.

The invention also features on other embodiments, an expression vector comprising a nucleic acid sequence encoding the chimeric firefly luciferase of any one of the above aspects and embodiments. In further related embodiments, the firefly luciferase is expressed from a mammalian codon optimized gene. In other further related embodiments, the expression vector further comprises a promoter sequence. In other embodiments, the invention features a cell comprising the expression vector.

In certain embodiments, the invention features a kit comprising the firefly luciferase of any one of the above aspects or embodiments.

The present invention also features a method for detection of transcriptional activity in a cell comprising introducing the expression vector of the above embodiments into a cell, wherein the expression vector comprises a promoter of interest, and detecting the light emission, wherein the detection of light emission indicates transcriptional activity.

The present invention also features a method for in vivo imaging comprising introducing the firefly luciferase of any one of the above claims into a cell of a living animal and detecting the light emission.

The present invention also features a method for detecting the amount of ATP in a sample comprising contacting a sample with a firefly luciferases of any one of the above claims; and detecting ATP.

These and other embodiments of the invention will become apparent in light of the detailed description below.

### BRIEF DESCRIPTION OF THE DRAWINGS

FIG. 1 shows an amino acid sequence alignment of P. pyralis luciferase (PpyWT, SEQ ID NO:4), L. italica luciferase (LitWT, SEQ ID NO:2) and PpyLit (SEQ ID NO:8). Figure discloses the consensus sequence as SEQ ID NO: 51.

FIG. 2 shows the cDNA (SEQ ID NO:7) and deduced amino acid sequence (SEQ ID NO:8) of PpyLit.

FIG. 3 shows the PpyS cDNA sequence (SEQ ID NO:9).

FIG. 4 shows the Ppy5 amino acid sequence (SEQ ID NO:10).

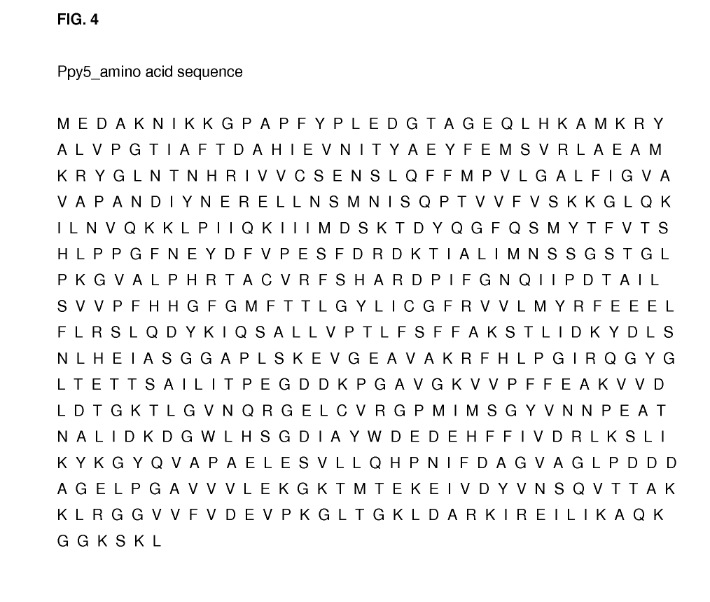

FIG. 5 shows the Ppy WT-Thermostable (TS) cDNA sequence (SEQ ID NO:21)

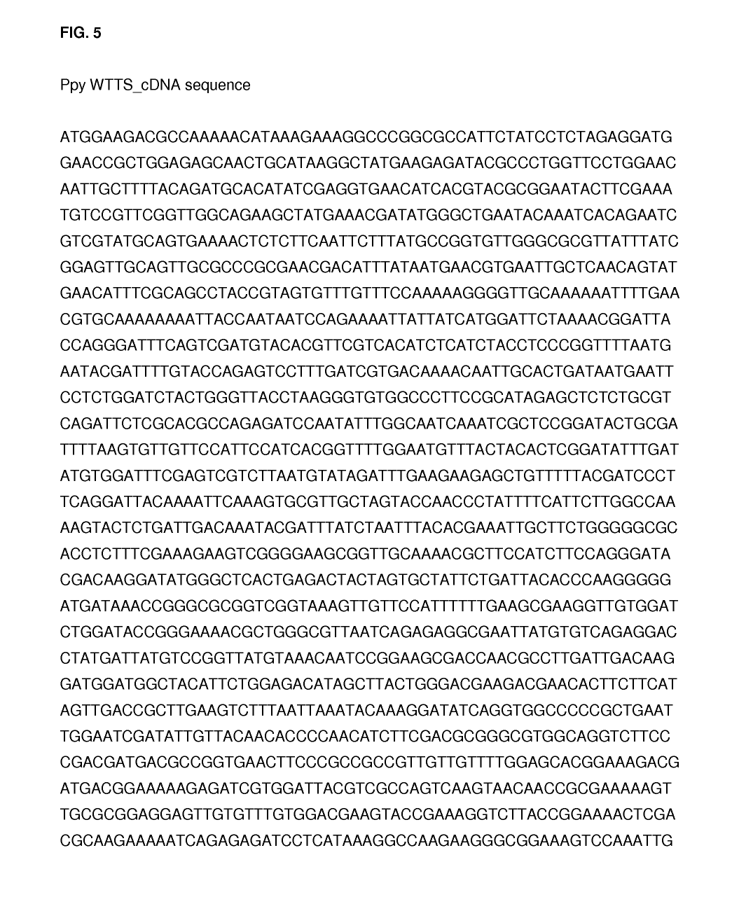

FIG. 6 shows the Ppy WT-TS amino acid sequence (SEQ ID NO:22)

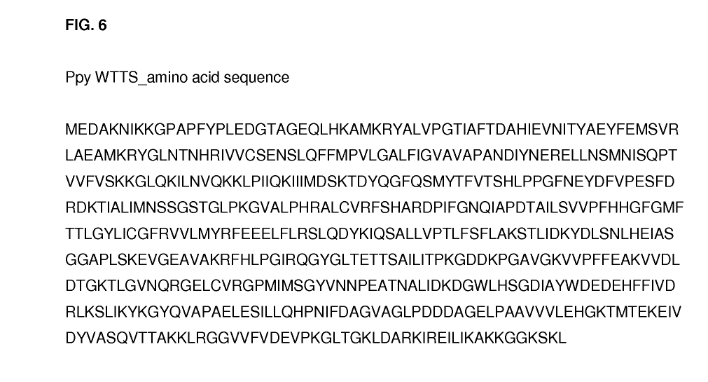

FIG. 7 shows the Ppy WT-TS5/I351V cDNA sequence (SEQ ID NO:11).

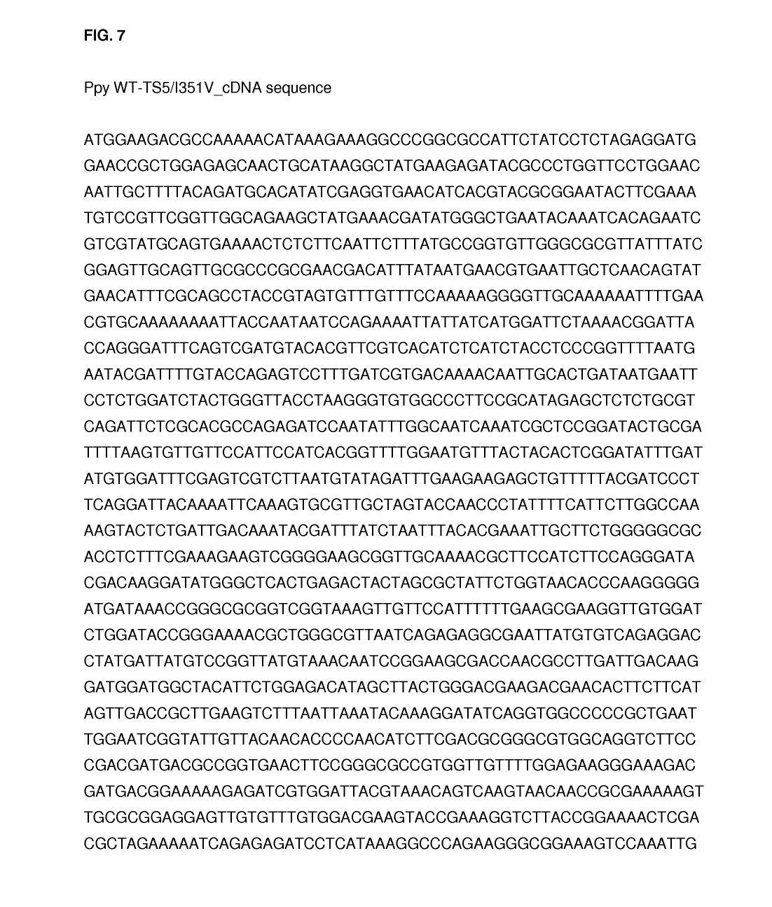

FIG. 8 shows the Ppy WT-TS5/I351V amino acid sequence (SEQ ID NO:12).

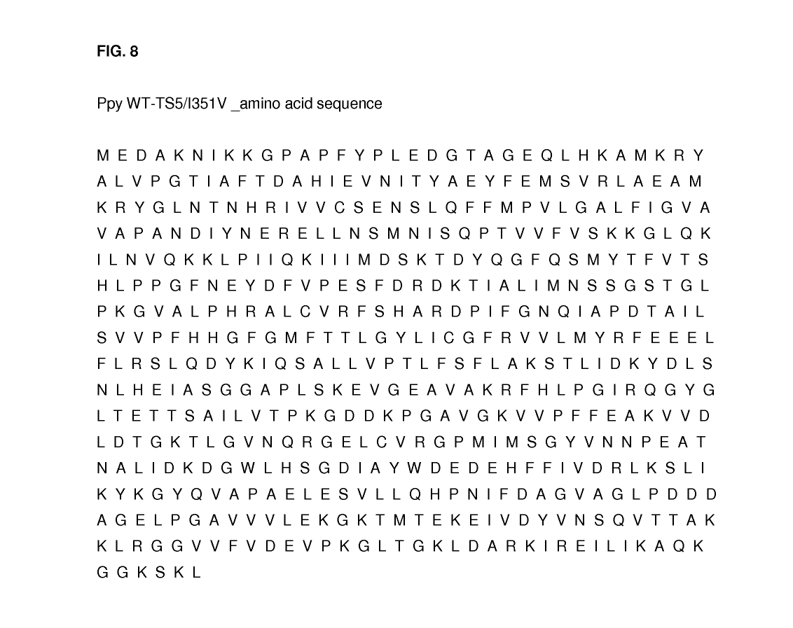

FIG. 9 shows the Ppy RE-TS cDNA sequence (SEQ ID NO:23)

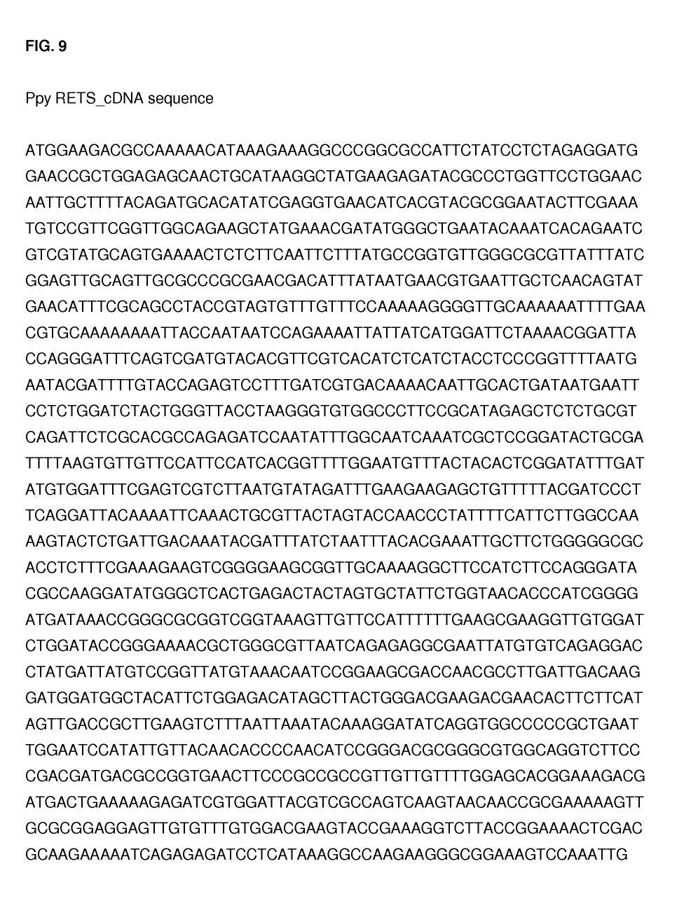

FIG. 10 shows the Ppy RE-TS cDNA sequence (SEQ ID NO:24)

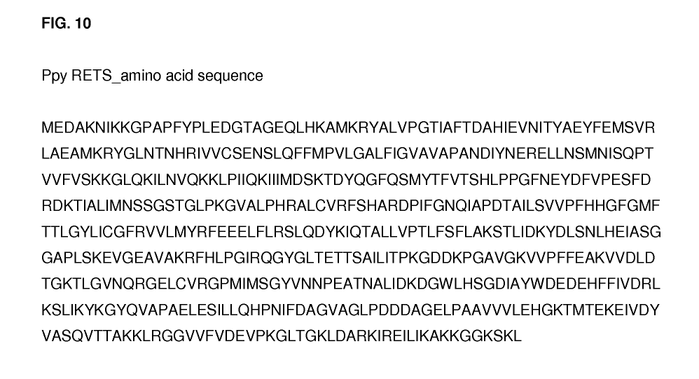

FIG. 11 shows the Ppy RE-TS5/I351V cDNA sequence (SEQ ID NO:13).

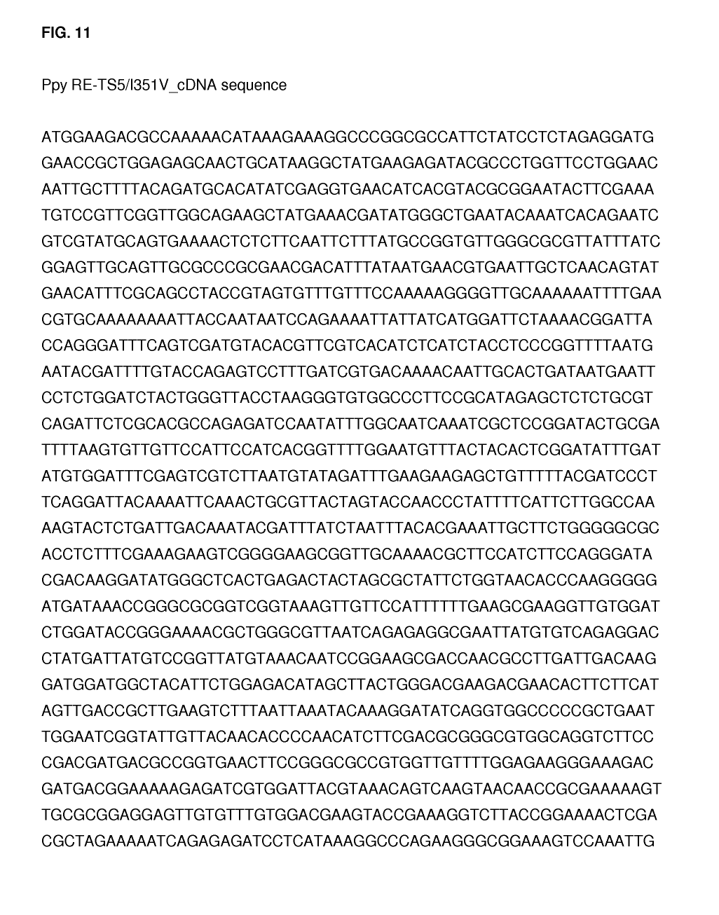

FIG. 12 shows the Ppy RE-TS5/I351V amino acid sequence (SEQ ID NO:14).

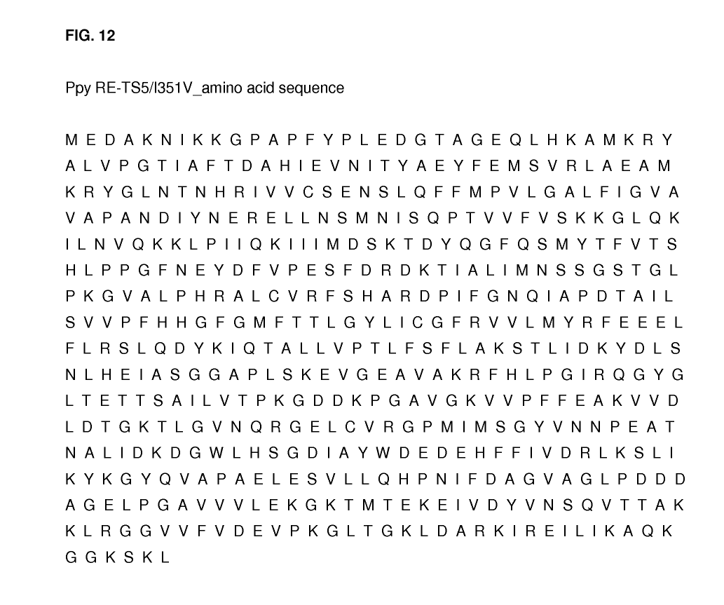

FIG. 13 shows the Ppy RE13 cDNA sequence (SEQ ID NO:15).

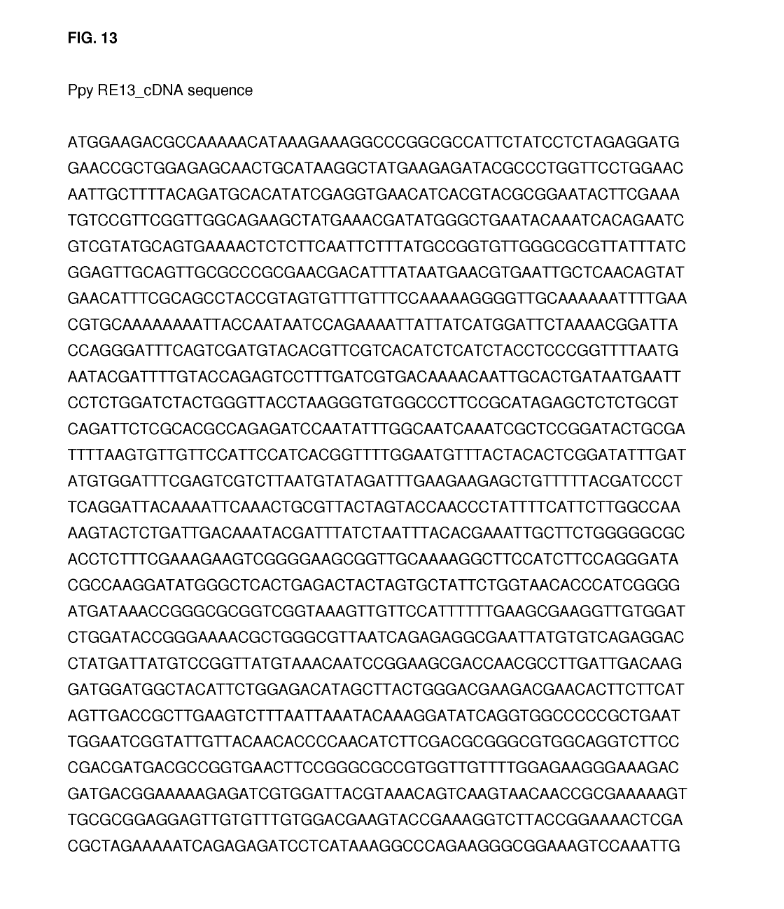

FIG. 14 shows the Ppy RE13 amino acid sequence (SEQ ID NO:16).

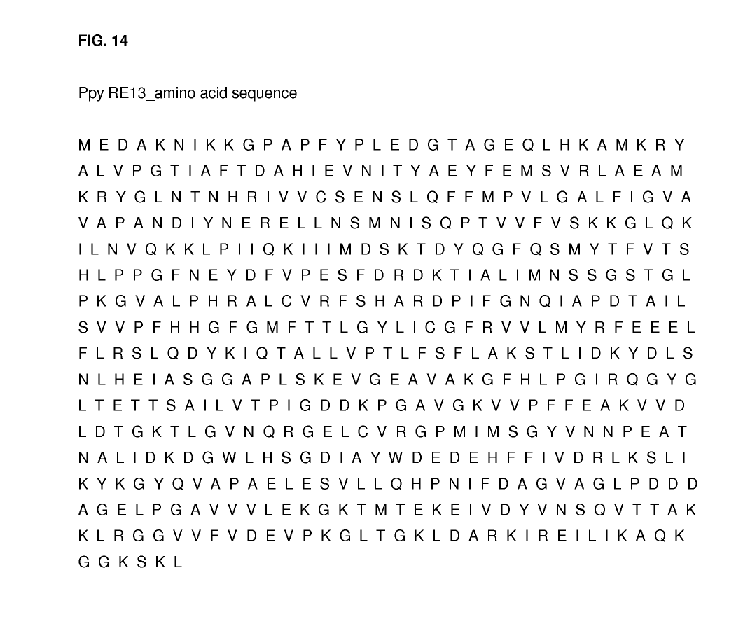

FIG. 15 shows the human codon optimized Ppy RE13 cDNA sequence (SEQ ID NO:17).

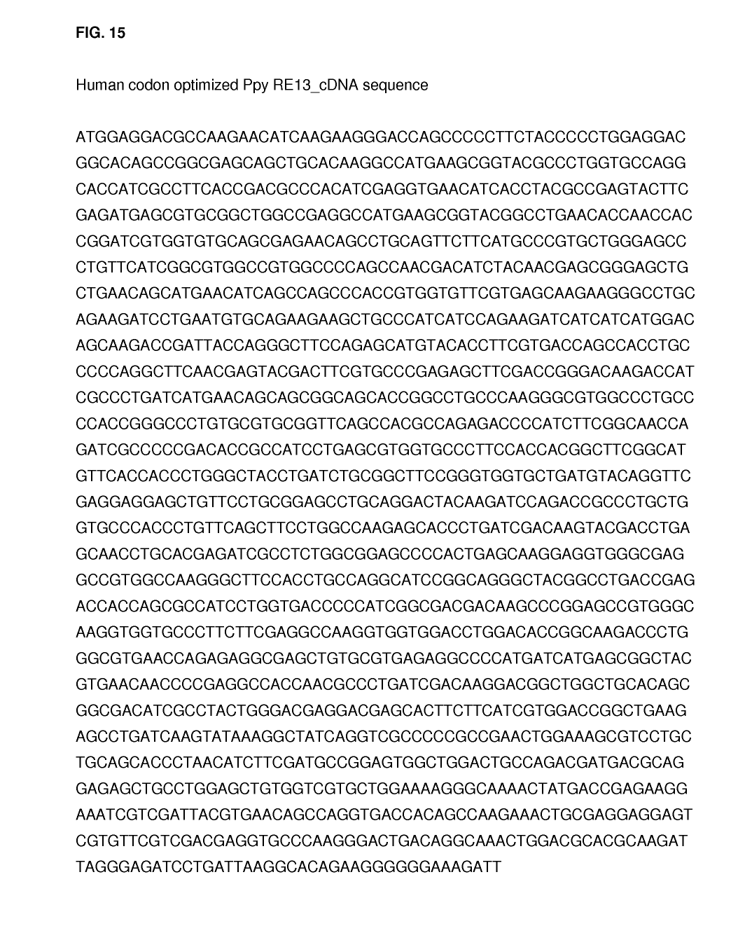

FIG. 16 shows the human codon optimized Ppy RE13 amino acid sequence (SEQ ID NO:18).

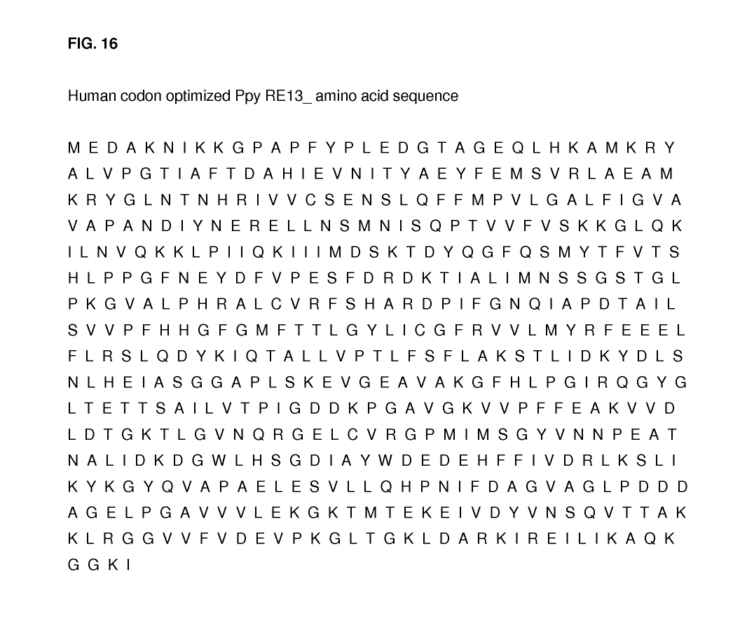

FIG. 17 shows the PpyRE9 cDNA sequence (SEQ ID NO:19)

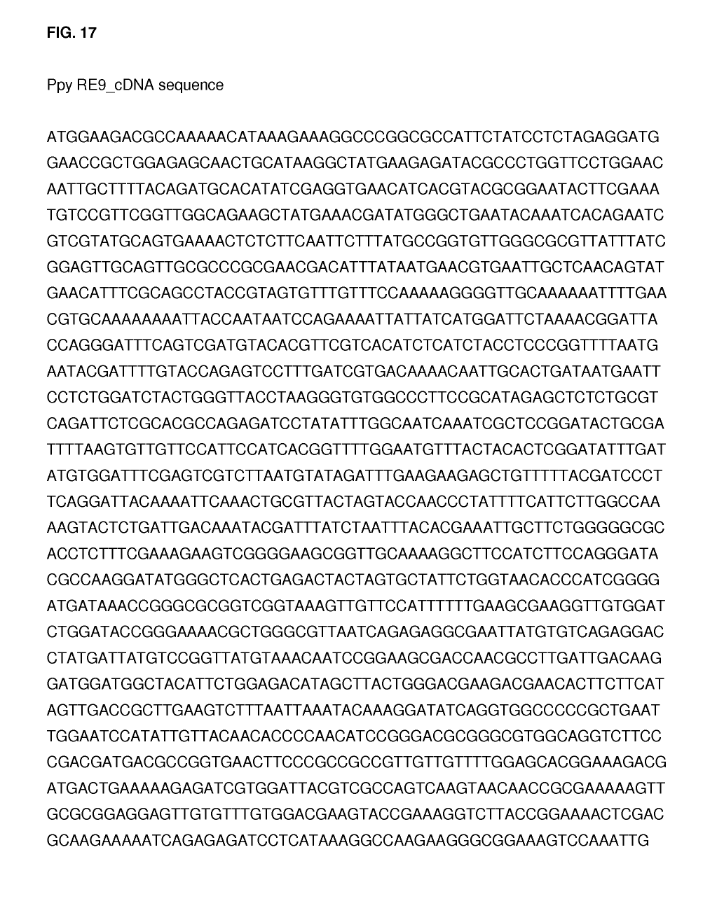

FIG. 18 shows the PpyRE9 amino acid sequence (SEQ ID NO:20)

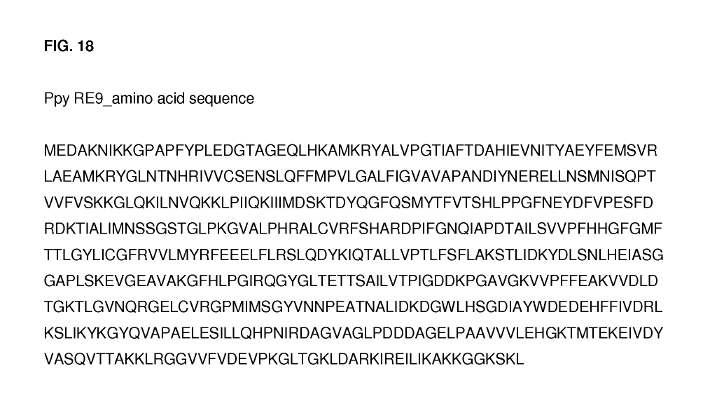

FIG. 19 shows the bioluminescence emission spectra as a function of pH. The normalized emission spectra for (A) Ppy WT, (B) PpyLit, (C) Ppy19, (D) PpyLit F465R, (E) PpyLit I351V/E354K, (F) PpyLit I232A/E354K, and (G) PpyLit I232A/I351V/E354K are shown at pH 7.8 ( [symbol] ) pH 7.0 ( - - - ), and pH 6.5 ( . . . ). In panel (H) PpyLit S284T ( [symbol] ) and PpyLit S284T/I351V/E354K ( - - - ) are shown representing pH 7.8, 7.0, and 6.5. Emission spectra produced by bioluminescence were obtained using a Horiba Jobin-Yvon iHR imaging spectrometer equipped with a liquid N2 cooled CCD detector and the excitation source turned off. Data were collected at 22° C. in a 0.8 mL quartz cuvette over the wavelength range 450-750 nm with the emission slit width set to 5 nm. Reactions (0.52 mL in 25 mM glycylglycine buffer pH 7.8, 25 mM Tris pH 7.0, or 25 mM MES pH 6.5) containing 100 μM firefly luciferin (LH₂) and 2 mM Mg-ATP were initiated by the addition of 5 μL of enzyme in CBA (0.02-0.03 μM final concentration). The pH values were confirmed before and after spectra were obtained. All spectra were corrected for the spectral response of the CCD using a correction curve provided by the manufacturer.

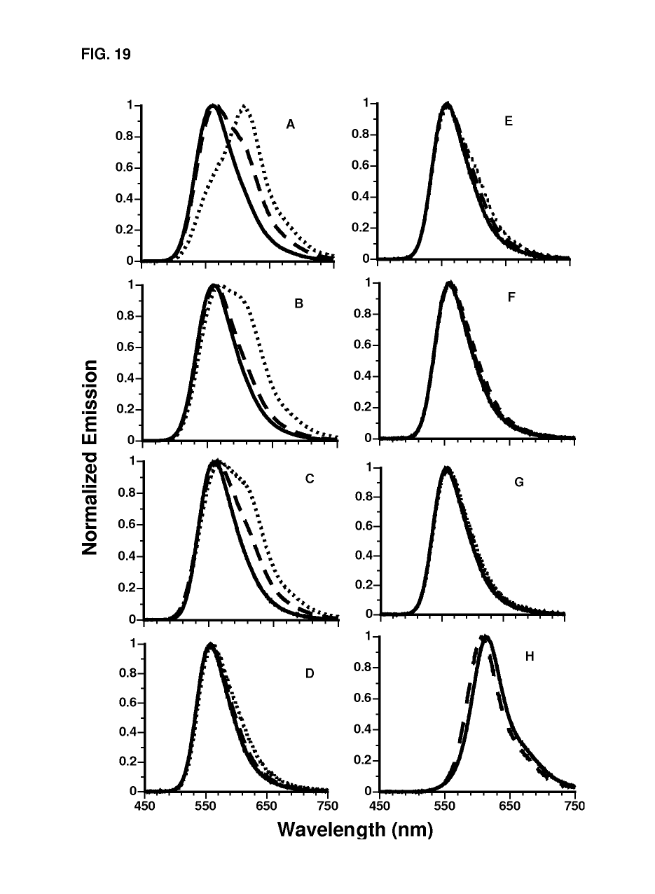

### DETAILED DESCRIPTION

### Definitions

Unless defined otherwise, all technical and scientific terms have the same meaning as is commonly understood by one of skill in the art to which this invention belongs.

The term cell” or “host cell” as used herein, are used interchangeably, and all such designations include progeny or potential progeny of these designations. A nucleic acid molecule of the invention may be introduced into a suitable cell line so as to create a stablytransfected cell line capable of producing the protein or polypeptide encoded by the gene. Vectors, cells, and methods for constructing such cell lines are well known in the art.

The term “codon optimization” is meant to refer to a technique used to improve the protein expression in living organism by increasing the translational efficiency of a gene of interest by transforming DNA sequence of nucleotides of one species into DNA sequence of nucleotides of another species.

The term “color shifting” as used herein is meant to refer to a change in the color of emitted light. The bioluminescence color of firefly luciferases is determined by the luciferase structure and assay conditions. In certain preferred embodiments, the color of emitted light is red. In other related embodiments, the emitted light is at a wavelength of 607 to 614 nm

The term “detection” as used herein, refers to quantitatively or qualitatively determining the light emission. The term “detection” can also refer to quantitatively or qualitatively determining the effect of a test compound on the sample.

The term “flash height activity” or “flash height based activity” as used herein is meant to refer to an assay used to determine luciferase bioluminescence activity. Flash height-based activity is a measure of the maximum achievable overall reaction rate as determined by measuring the maximum intensity of light produced under standard conditions in which an aliquot of enzyme is mixed with a saturating concentration of luciferin at pH ˜8, followed by rapid addition of a saturating concentration of MgATP. This measure is approximately equivalent to the standard biochemical measure of initial reaction velocity.

The term “integration activity,” “integration based activity” or “integration specific activity” as used herein is meant to refer to activities that reflect a measure of the total light emitted under similar standard conditions as above. It is usually necessary to collect light emission data for approximately 15 min (intensity values per time point are integrated) to collect ˜90% of the emitted light. This measure of activity is not dependent on the rate of light production as is the case for the flash height measurement.

“Flash height specific activity” and “integration specific activity” as used herein are meant to refer to enzyme activity that is measured by monitoring light emission (the peak height of the initial burst of light or total light emitted). Specific activity, a property of the enzymes, was determined using flash height or integration activity measurements and measurements of protein concentration. It is expressed as activity/amount of protein, typically activity units/mg.

The term “luciferase” as used herein, is meant to refer to one or more oxygenases that catalyze a light emitting reaction. Thus, luciferase refers to an enzyme or photoprotein that catalyzes a reaction that produces bioluminescence. Luciferases of the invention can be recombinant or naturally occurring, or a variant or mutant thereof, such as a variant produced by mutagenesis that has one or more properties, such as thermal stability, that differ from the naturally-occurring protein. Non-limiting examples of naturally occurring luciferases include, luciferases found among marine arthropods, firefly luciferase, click beetle luciferase, and railroad worm luciferase.

The term “nucleic acid molecule,” “polynucleotide,” or “nucleic acid sequence” as used herein, refers to nucleic acid, DNA or RNA, that comprises coding sequences necessary for the production of a polypeptide or protein precursor. The encoded polypeptide may be a full-length polypeptide, a fragment thereof (less than full-length), or a fusion of either the full-length polypeptide or fragment thereof with another polypeptide, yielding a fusion polypeptide.

By “peptide,” “protein” or “polypeptide” is meant any chain of amino acids, regardless of length or post-translational modification (e.g., glycosylation or phosphorylation). The nucleic acid molecules of the invention may also encode a variant of a naturally-occurring protein or polypeptide fragment thereof, which has an amino acid sequence that is at least 85%, 90%, 95% or 99% identical to the amino acid sequence of the naturally-occurring (native or wild-type) protein from which it is derived.

Polypeptide molecules are said to have an “amino terminus” (N-terminus) and a “carboxy terminus” (C-terminus) The terms “N-terminal” and “C-terminal” in reference to polypeptide sequences refer to regions of polypeptides including portions of the N-terminal and C-terminal regions of the polypeptide, respectively. A sequence that includes a portion of the N-terminal region of polypeptide includes amino acids predominantly from the N-terminal half of the polypeptide chain, but is not limited to such sequences. N-terminal and C-terminal regions may, but need not, include the amino acid defining the ultimate N-terminus and C-terminus of the polypeptide, respectively.

The term “sample” as used herein, is meant to refer to a cell or a population of cells, optionally in a growth media, or a cell lysate, a sample may also be a solid surface, (e.g., a swab, membrane, filter, particle), suspected of containing an attached cell or population of cells.

The term “vector” as used herein is meant to refer to nucleic acid molecules into which fragments of DNA may be inserted or cloned and can be used to transfer DNA segment(s) into a cell and capable of replication in a cell. Vectors may be derived from expression vectors, bacteriophages, viruses, cosmids, and the like. The terms “recombinant vector” and “expression vector” as used herein refer to DNA or RNA sequences containing a desired coding sequence and appropriate DNA or RNA sequences necessary for the expression of the operably linked coding sequence in a particular host organism. Prokaryotic expression vectors include a promoter, a ribosome binding site, an origin of replication for autonomous replication in a host cell and possibly other sequences, e.g. an optional operator sequence, optional restriction enzyme sites. A promoter is defined as a DNA sequence that directs RNA polymerase to bind to DNA and to initiate RNA synthesis. Eukaryotic expression vectors include a promoter, optionally a polyadenylation signal and optionally an enhancer sequence.

The term “wild-type” as used herein, is meant to refer to a gene or gene product that has the characteristics of that gene or gene product isolated from a naturally occurring source. A wild-type gene is that which is most frequently observed in a population and is thus arbitrarily designated the “wild-type” form of the gene.

### Luciferases

Luciferase enzymes, whose catalytic products include light, offer high sensitivity, a detectable product and enable easy measurement of ATP or other molecule such as luciferin or luciferin derivatives. At their most basic level, luciferases are defined by their ability to produce luminescence. More specifically, a luciferase is an enzyme that catalyzes the oxidation of a substrate, luciferin, thereby producing oxyluciferin and photons.

Since the earliest studies, beetle luciferases, particularly those from the common North American firefly species Photinus pyralis, have served as paradigms for understanding of bioluminescence. The fundamental knowledge and applications of luciferase have been based on a single enzyme, called “firefly luciferase,” derived from Photinus pyralis. However, there are roughly 1800 species of luminous beetles worldwide.

Beetle luciferases comprise one major group of the ANL superfamily of adenylating enzymes. This superfamily also includes the acyl-CoA synthetases and the adenylation domains of the nonribososmal peptide synthetases (NRPSs). The two domain structure of the superfamily enzymes that share ˜20% sequence identity was originally recognized by Brick and coworkers in their seminal crystallographic study in which they identified the Photinus pyralis luciferase (Luc) large N-domain (residues 1-436) and small C-domain (residues 440-550) connected through the short hinge region 437ArgLeuLys439. According to the well documented domain alternation mechanism originally proposed by Gulick, the ANL superfamily enzymes catalyze two half-reactions as shown below in Scheme 1.

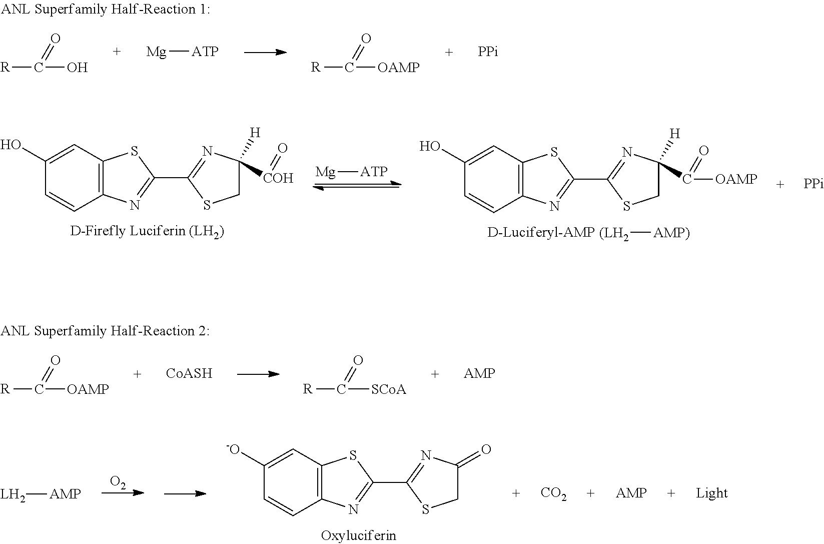

First a carboxylate-containing substrate is converted into the corresponding adenylate in a reaction requiring ATP. With the exception of the luciferases, the second half-reaction is characterized by the substitution of AMP by a thiol, typically coenzyme A (CoA), producing a thioester product. Instead the second luciferase-catalyzed reaction is multistep oxidative process that produces light, as shown above. Luciferase can, however, use CoA to convert dehydroluciferyl-AMP (L-AMP), a potent inhibitor formed in a dark side reaction, into L-CoA slowly releasing active enzyme.

Firefly (Photinus pyralis) luciferase is a monomeric enzyme of 61 kDa that requires no posttranslational modifications for activity. It acts by first combining beetle luciferin with ATP, to form luciferyl-AMP as an enzymebound intermediate. This intermediate reacts with O2 to create another bound intermediate, oxyluciferin, in a high-energy state. The subsequent energy transition to the ground state yields yellow-green light with a spectral maximum of 560 nm.

Renilla luciferase from the sea pansy Renilla reniformis is a 36-kDa monomeric enzyme that catalyzes the oxidation of coelenterazine to yield coelenteramide and blue light with a spectral maximum of 480 nm. It has been used primarily as a co-reporter in conjunction with firefly luciferase.

Other luciferases that have been introduced as candidates for genetic reporters include click beetle, Gaussia, Metridia, and Vargula luciferases.

The present invention is based on the finding that a chimeric luciferase (PpyLit), which catalyzes yellow-green light emission (560 nm maximum), and is comprised of the N-domain (residues 1-436) of recombinant P. pyralis luciferase (PpyWT) joined to the C-domain of Luciola italica luciferase (LitWT) had unusually enhanced properties compared to wild type luciferases (native luciferase, recombinant P. pyralis and recombinant L. italica).

The nucleic acid sequence of wild type L. italica (LitWT) is shown below, as SEQ ID NO:1.

SEQ ID NO: 1
(LitWT nucleic acid sequence)
ATGGAAACGGAAAGGGAGGAAAATGTTGTATATGGCCCTCTGCCATTCTA
CCCCATTGAAGAAGGATCAGCTGGAATTCAGTTGCATAAGTACATGCAAC
AATATGCCAAACTTGGAGCAATTGCTTTTAGTAACGCCCTTACTGGAGTG
GATATTTCTTACCAACAATACTTTGATATTACATGTCGTTTAGCTGAGGC
AATGAAAAACTACGGTATGAAACCGGAAGGACATATTGCTTTGTGCAGTG
AAAATTGTGAAGAATTTTTCATCCCTGTGCTTGCTGGTCTTTACATTGGA
GTAACTGTCGCACCTACTAATGAAATTTACACATTGCGTGAACTTAATCA
CAGTTTGGGCATCGCACAACCAACTATTGTATTCAGCTCCAGAAAAGGCT
TACCTAAAGTTTTAGAAGTGCAAAAAACAGTTACATGCATCAAAACAATT
GTTATTTTAGATAGTAAAGTAAACTTTGGAGGCTACGATTGTGTGGAAAC
TTTTATTAAGAAACATGTAGAATTAGGTTTTCCAGCAACTAGCTTTGTAC
CCATTGATGTAAAGGACCGTAAACATCACATTGCTTTGCTTATGAATTCT
TCTGGCTCTACTGGTTTACCTAAAGGTGTAGAGATTACCCACGAAGGAAC
AGTTACAAGATTCTCACACGCTAAGGATCCAATTTACGGAAACCAAGTTT
CACCTGGTACTGCTATTTTAACTGTCGTTCCGTTCCATCATGGATTTGGA
ATGTTTACCACTTTAGGATACTTTGCTTGTGGATACCGTATTGTAATGTT
AACAAAATTCGATGAAGAACTATTTTTGAGAACTTTGCAAGATTATAAGT
GTACCAGTGTTATTCTTGTACCAACGTTATTTGCTATTCTCAACAGGAGT
GAATTGCTCGATAAGTTCGATTTATCTAATCTAACTGAAATTGCTTCTGG
TGGAGCTCCTTTGGCAAAAGAAATTGGTGAAGCAGTCGCTAGAAGATTTA
ATCTACCCGGTGTCCGTCAGGGTTACGGATTGACAGAAACGACATCTGCA
TTTATTATTACCCCAGAAGGTGATGATAAACCTGGAGCATCTGGAAAAGT
AGTACCCTTATTCAAAGTAAAAATTATTGATCTTGACACTAAAAAAACTT
TGGGTGTCAACCGACGAGGAGAGATCTGTGTAAAAGGTCCGAGTCTTATG
TTAGGCTACACAAACAATCCGGAAGCAACAAGAGAAACTATTGATGAAGA
GGGTTGGTGCACACCGGAGATATTGGATATTACGACGAAGACGAACATTT
CTTCATTGTAGATCGTTTGAAATCATTAATCAAATACAAGGGGTACCAGG
TACCACCTGCTGAATTGGAATCCGTTCTTTTGCAACATCCAAATATCTTT
GATGCTGGTGTGGCTGGTGTCCCCGATTCTGAAGCTGGTGAACTTCCAGG
GGCTGTAGTTGTAATGGAAAAAGGAAAAACTATGACTGAAAAGGAAATTG
TGGATTATGTTAATAGTCAAGTAGTGAACCACAAACGTCTGCGTGGTGGC
GTTCGTTTTGTGGATGAAGTACCTAAAGGTCTAACTGGTAAAATTGATGC
TAAAGTAATTAGAGAAATTCTTAAGAAACCACAAGCCAAGATG

The corresponding wild type L. italica (LitWT) amino acid sequence is shown below as SEQ ID NO:2.

SEQ ID NO: 2
(LitWT amino acid sequence)
M E T E R E E N V V Y G P L P F Y P I E E G S A G
I Q L H K Y M Q Q Y A K L G A I A F S N A L T G V
D I S Y Q Q Y F D I T C R L A E A M K N Y G M K P
E G H I A L C S E N C E E F F I P V L A G L Y I G
V T V A P T N E I Y T L R E L N H S L G I A Q P T
I V F S S R K G L P K V L E V Q K T V T C I K T I
V I L D S K V N F G G Y D C V E T F I K K H V E L
G F P A T S F V P I D V K D R K H H I A L L M N S
S G S T G L P K G V E I T H E G T V T R F S H A K
D P I Y G N Q V S P G T A I L T V V P F H H G F G
M F T T L G Y F A C G Y R I V M L T K F D E E L F
L R T L Q D Y K C T S V I L V P T L F A I L N R S
E L L D K F D L S N L T E I A S G G A P L A K E I
G E A V A R R F N L P G V R Q G Y G L T E T T S A
F I I T P E G D D K P G A S G K V V P L F K V K I
I D L D T K K T L G V N R R G E I C V K G P S L M
L G Y T N N P E A T R E T I D E E G W L H T G D I
G Y Y D E D E H F F I V D R L K S L I K Y K G Y Q
V P P A E L E S V L L Q H P N I F D A G V A G V P
D S E A G E L P G A V V V M E K G K T M T E K E I
V D Y V N S Q V V N H K R L R G G V R F V D E V P
K G L T G K I D A K V I R E I L K K P Q A K M

In certain preferred embodiments, the nucleic acid sequence of wild type L. italica comprises mutations to eliminate the C-terminal AKM peroxisome signal. This is shown below as SEQ ID NO:5. In SEQ ID NO:5, the C-terminal AKM peroxisome signal it is changed to AGG. According to preferred embodiments of the present invention, PpyLit and mutants all contain the AGG terminus

SEQ ID NO: 5
(LitWT AGG peptide nucleic acid sequence)
ATGGAAACGGAAAGGGAGGAAAATGTTGTATATGGCCCTCTGCCATTCTA
CCCCATTGAAGAAGGATCAGCTGGAATTCAGTTGCATAAGTACATGCAAC
AATATGCCAAACTTGGAGCAATTGCTTTTAGTAACGCCCTTACTGGAGTG
GATATTTCTTACCAACAATACTTTGATATTACATGTCGTTTAGCTGAGGC
AATGAAAAACTACGGTATGAAACCGGAAGGACATATTGCTTTGTGCAGTG
AAAATTGTGAAGAATTTTTCATCCCTGTGCTTGCTGGTCTTTACATTGGA
GTAACTGTCGCACCTACTAATGAAATTTACACATTGCGTGAACTTAATCA
CAGTTTGGGCATCGCACAACCAACTATTGTATTCAGCTCCAGAAAAGGCT
TACCTAAAGTTTTAGAAGTGCAAAAAACAGTTACATGCATCAAAACAATT
GTTATTTTAGATAGTAAAGTAAACTTTGGAGGCTACGATTGTGTGGAAAC
TTTTATTAAGAAACATGTAGAATTAGGTTTTCCAGCAACTAGCTTTGTAC
CCATTGATGTAAAGGACCGTAAACATCACATTGCTTTGCTTATGAATTCT
TCTGGCTCTACTGGTTTACCTAAAGGTGTAGAGATTACCCACGAAGGAAC
AGTTACAAGATTCTCACACGCTAAGGATCCAATTTACGGAAACCAAGTTT
CACCTGGTACTGCTATTTTAACTGTCGTTCCGTTCCATCATGGATTTGGA
ATGTTTACCACTTTAGGATACTTTGCTTGTGGATACCGTATTGTAATGTT
AACAAAATTCGATGAAGAACTATTTTTGAGAACTTTGCAAGATTATAAGT
GTACCAGTGTTATTCTTGTACCAACGTTATTTGCTATTCTCAACAGGAGT
GAATTGCTCGATAAGTTCGATTTATCTAATCTAACTGAAATTGCTTCTGG
TGGAGCTCCTTTGGCAAAAGAAATTGGTGAAGCAGTCGCTAGAAGATTTA
ATCTACCCGGTGTCCGTCAGGGTTACGGATTGACAGAAACGACATCTGCA
TTTATTATTACCCCAGAAGGTGATGATAAACCTGGAGCATCTGGAAAAGT
AGTACCCTTATTCAAAGTAAAAATTATTGATCTTGACACTAAAAAAACTT
TGGGTGTCAACCGACGAGGAGAGATCTGTGTAAAAGGTCCGAGTCTTATG
TTAGGCTACACAAACAATCCGGAAGCAACAAGAGAAACTATTGATGAAGA
GGGTTGGTTGCACACCGGAGATATTGGATATTACGACGAAGACGAACATT
TCTTCATTGTAGATCGTTTGAAATCATTAATCAAATACAAGGGGTACCAG
GTACCACCTGCTGAATTGGAATCCGTTCTTTTGCAACATCCAAATATCTT
TGATGCTGGTGTGGCTGGTGTCCCCGATTCTGAAGCTGGTGAACTTCCAG
GGGCTGTAGTTGTAATGGAAAAAGGAAAAACTATGACTGAAAAGGAAATT
GTGGATTATGTTAATAGTCAAGTAGTGAACCACAAACGTCTGCGTGGTGG
CGTTCGTTTTGTGGATGAAGTACCTAAAGGTCTAACTGGTAAAATTGATG
CTAAAGTAATTAGAGAAATTCTTAAGAAACCACAAGCCGGGGGG

The corresponding wild type L. italica amino acid sequence with mutations to eliminate the C-terminal AKM peroxisome signal is shown below as SEQ ID NO:6.

SEQ ID NO: 6
(LitWT AGG peptide amino acid sequence)
M E T E R E E N V V Y G P L P F Y P I E E G S A G
I Q L H K Y M Q Q Y A K L G A I A F S N A L T G V
D I S Y Q Q Y F D I T C R L A E A M K N Y G M K P
E G H I A L C S E N C E E F F I P V L A G L Y I G
V T V A P T N E I Y T L R E L N H S L G I A Q P T
I V F S S R K G L P K V L E V Q K T V T C I K T I
V I L D S K V N F G G Y D C V E T F I K K H V E L
G F P A T S F V P I D V K D R K H H I A L L M N S
S G S T G L P K G V E I T H E G T V T R F S H A K
D P I Y G N Q V S P G T A I L T V V P F H H G F G
M F T T L G Y F A C G Y R I V M L T K F D E E L F
L R T L Q D Y K C T S V I L V P T L F A I L N R S
E L L D K F D L S N L T E I A S G G A P L A K E I
G E A V A R R F N L P G V R Q G Y G L T E T T S A
F I I T P E G D D K P G A S G K V V P L F K V K I
I D L D T K K T L G V N R R G E I C V K G P S L M
L G Y T N N P E A T R E T I D E E G W L H T G D I
G Y Y D E D E H F F I V D R L K S L I K Y K G Y Q
V P P A E L E S V L L Q H P N I F D A G V A G V P
D S E A G E L P G A V V V M E K G K T M T E K E I
V D Y V N S Q V V N H K R L R G G V R F V D E V P
K G L T G K I D A K V I R E I L K K P Q A G G

The nucleic acid sequence of wild type P. pyralis (PpyWT) is shown below, as SEQ ID NO:3.

SEQ ID NO: 3
(PpyWT nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGATATTGTTACAACACCCCAACATCTTCGACGC
GGGCGTGGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG

The corresponding wild type P. pyralis (PpyWT) amino acid sequence is shown below as SEQ ID NO:4.

SEQ ID NO: 4
(PpyWT amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R T A C V R F S H A R D P
I F G N Q I I P D T A I L S V V P F H H G F G M F
T T L G Y L I C G F R V V L M Y R F E E E L F L R
S L Q D Y K I Q S A L L V P T L F S F F A K S T L
I D K Y D L S N L H E I A S G G A P L S K E V G E
A V A K R F H L P G I R Q G Y G L T E T T S A I L
I T P E G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S I L L Q H P N I F D A G V A G L P D D
D A G E L P A A V V V L E H G K T M T E K E I V D
Y V A S Q V T T A K K L R G G V V F V D E V P K G
L T G K L D A R K I R E I L I K A K K G G K S K L

Full length luciferase, fragments of luciferase (e.g. the N-terminal amino acid domain or the C-terminal amino acid domain), variants of luciferase, and variant fragments of luciferase enzyme used in the compositions and methods of the present invention may be purified from a native source or prepared by a number of techniques, including (1) chemical synthesis, (2) enzymatic (protease) digestion of luciferase, and (3) recombinant DNA methods. Chemical synthesis methods are well known in the art, as are methods that employ proteases to cleave specific sites. To produce segments of luciferase protein segments of luciferase or luciferase variants can be made and then expressed in a host organism, such as E. coli. Methods such as endonuclease digestion or polymerase chain reaction (PCR) allow one of skill in the art to generate an unlimited supply of well-defined fragments.

Accordingly, in one aspect the present invention features a chimeric firefly luciferase comprising an N-terminal amino acid domain from a first firefly luciferase and a C-terminal amino acid domain from a second firefly luciferase. In certain embodiments, the N-terminal amino acid domain is from P. pyralis luciferase and the C-terminal amino acid domain is from L. italica luciferase.

According to certain embodiments, the L. italica luciferase comprises the amino acid sequence of SEQ ID NO:2 or SEQ ID NO:6. In other related embodiments, the C-terminal amino acid domain comprises the amino acid sequence of SEQ ID NO:2 or SEQ ID NO:6 and the N-terminal amino acid domain is from a second firefly luciferase. Preferably, the N-terminal amino acid domain is from P. pyralis luciferase.

According to other embodiments, the P. pyralis luciferase comprises the amino acid sequence of SEQ ID NO:4. In other related embodiments, the N-terminal amino acid domain comprises the amino acid sequence of SEQ ID NO:4 and the C-terminal amino acid domain is from a second firefly luciferase. Preferably, the C-terminal amino acid domain is from L. italica luciferase.

The domain nomenclature dates back to the first crystal structure paper by Conti and Brick (Conti, E., Franks, N. P., and Brick, P. (1996) “Crystal structure of firefly luciferase throws light on a superfamily of adenylate-forming enzymes”, Structure 4, 287-298; incorporated by reference in its entirety herein), and is based on PpyWT. This paper described the N-Domain as residues 4-436, the C-Domain as 440-544 and the connecting linker peptide as 437-439 (ArgLeuLys). However, because the x-ray data could not resolve residues 1-3 and 545-550, the N- and C-Domains are herein defined as residues 1-436 and 440-550, respectively corresponding to PpyWT numbering (SEQ ID NO:4).

Accordingly, in exemplary embodiments of the present invention, the N-terminal domain comprises residues 1-436 of SEQ ID NO: 4, and the C-terminal domain comprises residues 440-550 of SEQ ID NO:4.

It is contemplated in certain preferred embodiments, that the chimeric firefly luciferases of the present invention further comprise a linker peptide. Preferably, the linker peptide connects the N- and C-Domains. In certain preferred embodiments, the linker peptide is a tripeptide linker. In exemplary embodiments, the linker peptide comprises ArgLeuLys or ArgTyrLys.

For the ˜25 amino acid sequences of beetle luciferases that are available, the linker sequence ArgLeuLys is not absolutely conserved; however all the beetle luciferases have the sequence Arg-Leu/Tyr-Lys, because several click beetle luciferases have Tyr in place of Leu at position 438 (corresponding to SEQ ID NO:4 PpyWT).

The linker peptide may further comprise a mutation at any one of the amino acid residues.

Preferably, the linker peptide comprises residues 437-439 of SEQ ID NO:4.

Any type of amino acid substitution, insertion or deletion, or combination thereof may be used to generate a variant luciferase. A substitution may be a conservative or a non-conservative amino acid substitution. Conservative substitutions refer to an amino acid of one class being replaced with another amino acid of the same type. Non-conservative substitutions affect (1) the structure of the polypeptide backbone, such as a beta-sheet or α-helical conformation, (2) the charge or (3) hydrophobicity, or (4) the bulk of the side chain of the target site. Residues are divided into groups based on common side-chain properties. Non-conservative substitutions entail exchanging a member of one of these classes for another class.

Variant luciferase genes or gene fragments can be made using methods known in the art such as oligonucleotide-mediated (site-directed) mutagenesis, alanine scanning, and PCR mutagenesis. Site-directed mutagenesis, cassette mutagenesis, restriction selection mutagenesis or other know techniques can be performed on the cloned DNA to produce the luciferase variant DNA.

In preferred embodiments, the L italica luciferase comprises the amino acid sequence of SEQ ID NO:2 or SEQ ID NO:6. In further preferred embodiments, SEQ ID NO:2 is encoded by the nucleic acid sequence of SEQ ID NO: 1. In other further preferred embodiments, SEQ ID NO:6 is encoded by the nucleic acid sequence of SEQ ID NO: 5.

In preferred embodiments, the C-terminal amino acid domain comprises the amino acid sequence of SEQ ID NO:2 or SEQ ID NO:6, and the N-terminal amino acid domain is from a second firefly luciferase. In further preferred embodiments, the N-terminal amino acid domain is from P. pyralis luciferase.

In preferred embodiments, the P. pyralis luciferase comprises the amino acid sequence of SEQ ID NO:4. In further preferred embodiments, SEQ ID NO:4 is encoded by the nucleic acid sequence of SEQ ID NO:3.

In preferred embodiments, the N-terminal amino acid domain comprises the amino acid sequence of SEQ ID NO:4 and the C-terminal amino acid domain is from a second firefly luciferase. In related preferred embodiments, the C-terminal amino acid domain is from L. italica luciferase.

In preferred embodiments, the N-terminal domain comprises residues 1-436 of SEQ ID NO: 4, and the C-terminal domain comprises residues 440-550 of SEQ ID NO:6.

In other preferred embodiments, the chimeric firefly luciferase comprises the nucleic acid sequence of SEQ ID NO:7. In still other preferred embodiments, the chimeric firefly luciferase comprises the amino acid sequence of SEQ ID NO:8.

The present invention also features a firefly luciferase comprising the amino acid sequence of SEQ ID NO:4. In preferred embodiments, the firefly luciferase comprising the amino acid sequence of SEQ ID NO:4 further comprises one or more amino acid changes selected from the group consisting of: A450P, I457V, L472V, D475S, D476E, A482G, L487M, H489K, A503N, T507V, T508N, A509H, K511R and V517R. This may also be referred to as Ppy14.

In other preferred embodiments, the firefly luciferase comprising the amino acid sequence of SEQ ID NO:4 further comprises one or more amino acid changes selected from the group consisting of: A450P, I457V, L472V, D475S, D476E, A482G, L487M, H489K, A503N, T507V, T508N, A509H, K511R, V517R, L530I, K534V, 1540K, A542P and K543Q. This may also be referred to as Ppy19.

In other preferred embodiments, the firefly luciferase comprising the amino acid sequence of SEQ ID NO:4 further comprises an amino acid change I457V/A482G/H489K/A503N. This may also be referred to as Ppy4. The flash height specific activity of this variant is 157±10 (compared to PpyWT=100%) and the integration based value is 172±18. These values are ˜87% of those found in PpyLit. It appears however that this enzyme has thermostability properties (˜20-25 min to 50%) similar to PpyWT, ie, it is more thermostable than PpyLit.

In other further embodiments, an additional (5th) mutation K543Q has been introduced into this variant (Ppy 4). DNA sequencing confirmed the introduction of the 5th change. This may also be referred to as Ppy5. Ppy5 is Ppy4 plus the K543Q mutation. This enzyme has the full pH resistance associated with PpyLit. The flash height specific activity is 163±8 and the integration sp activity is 176±17. These values are 91% and 88%, respectively, of the PpyLit values. This protein too appears to have thermostability properties (˜20-25 min to 50%) similar to PpyWT, i.e., it is more thermostable than PpyLit.

The present invention also features a firefly luciferase comprising the amino acid sequence of SEQ ID NO:8. In preferred embodiments, the firefly luciferase comprising the amino acid sequence of SEQ ID NO:8 further comprises one or more amino acid changes.

For example, in related embodiments, the firefly luciferase comprising the amino acid sequence of SEQ ID NO:8 further comprises the amino acid change F465R. In preferred embodiments, the firefly luciferase comprising the amino acid sequence of SEQ ID NO:8 further comprises an amino acid change I232A/E354K. In other preferred embodiments, the firefly luciferase comprising the amino acid sequence of SEQ ID NO:8 further comprises an amino acid change I351V/E354K. In other preferred embodiments, the firefly luciferase comprising the amino acid sequence of SEQ ID NO:8 further comprises an amino acid change I232A/I351V/E354K. In other preferred embodiments, the firefly luciferase comprising the amino acid sequence of SEQ ID NO:8 further comprises an amino acid change I351V/E354K/F465R. In other preferred embodiments, the firefly luciferase comprising the amino acid sequence of SEQ ID NO:8 further comprises the amino acid change S284T. In other preferred embodiments, the firefly luciferase comprising the amino acid sequence of SEQ ID NO:8 further comprises the amino acid change S284T/F465R. In other preferred embodiments, the firefly luciferase comprising the amino acid sequence of SEQ ID NO:8 further comprises the amino acid change S284T/I351V/E354K. In other preferred embodiments, the firefly luciferase comprising the amino acid sequence of SEQ ID NO:8 further comprises the amino acid change I232A/S284T/I351V/E354I. In other preferred embodiments, the firefly luciferase further comprises the amino acid change S284T/I351V/E354K/F465R. In other preferred embodiments, the firefly luciferase comprising the amino acid sequence of SEQ ID NO:8 further comprises the amino acid change I232A/S284T/I351V/E354K/F465R.

In further preferred embodiments, the firefly luciferase comprising the amino acid sequence of SEQ ID NO:8 further comprises one or more amino acid changes selected from the group consisting of F465R, I232A, E354K, I351V, I232A, S284T, E354I, T214A, A215L, and F295L.

In preferred embodiments, the firefly luciferase comprising the amino acid sequence of SEQ ID NO:8 further comprises the amino acid change T214A/A215L/I232A/V241I/G246A/F250S/F295L/E354K.

In other preferred embodiments, the firefly luciferase comprising the amino acid sequence of SEQ ID NO:8 further comprises the amino acid change T214A/A215L/I232A/S284T/F295L/E354K.

In further preferred embodiments, the firefly luciferase comprising the amino acid sequence of SEQ ID NO:8 further comprises one or more amino acid changes selected from the group consisting of T214A, A215L, I232A, V241I, G246A, F250S, F295L, E354K, S284T and I351V.

The chimeric luciferases of the present invention may comprise an N-terminal extension peptide.

In certain exemplary embodiments, the wild type L. italica may have an N-terminal extension peptide that is GPLGS (SEQ ID NO: 25). However, the N-terminal extension peptide is not limited as such. In certain embodiments, no extension is needed, or another N-terminal extension known to one skilled in the art, such as one of the HisTag sequences, may also be used.

In other exemplary embodiments, the wild type P. pyralis may have an N-terminal extension peptide that is GPLGS (SEQ ID NO: 25). However, the N-terminal extension peptide is not limited as such. In certain embodiments, no extension is needed, or another N-terminal extension known to one skilled in the art, such as one of the HisTag sequences, may also be used.

In other embodiments of the present invention, it may be the case, that the full extent of the enhanced properties observed with the chimeric proteins depends on the PpyLit proteins being expressed as GST-fusion proteins and then cleaved leaving the GPLGS (SEQ ID NO: 25) extension. However, it is not the extension that it is important, it is the expression of the proteins as GST-fusion proteins that is relevant.

In certain embodiments, luciferases of the invention are resistant to red shifting of light emission at low pH. In preferred embodiments, exemplary chimeric luciferases of the present invention have the ability to emit red light at a wavelength of about 607-614 nm. In other preferred embodiments, exemplary chimeric luciferases have increased stability compared to P. pyralis wild type luciferase (PpyWT). In other preferred embodiments, exemplary chimeric luciferases have increased resistance to color shifting compared to P. pyralis wild type luciferase (PpyWT). In some embodiments, the flash height activity, integration specific activity or catalytic efficiency of the chimeric luciferases is increased compared to P. pyralis wild type luciferase (PpyWT).

An exemplified chimeric luciferase comprises the nucleic acid sequence of SEQ ID NO:7 and the corresponding deduced amino acid sequence, SEQ ID NO:8, shown below.

Nucleotides 1-1,638 of SEQ ID NO: 7 (PpyLit
nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAGGGGTACCAGGTACCA
CCTGCTGAATTGGAATCCGTTCTTTTGCAACATCCAAATATCTTTGATGC
TGGTGTGGCTGGTGTCCCCGATTCTGAAGCTGGTGAACTTCCAGGGGCTG
TAGTTGTAATGGAAAAAGGAAAAACTATGACTGAAAAGGAAATTGTGGAT
TATGTTAATAGTCAAGTAGTGAACCACAAACGTCTGCGTGGTGGCGTTCG
TTTTGTGGATGAAGTACCTAAAGGTCTAACTGGTAAAATTGATGCTAAAG
TAATTAGAGAAATTCTTAAGAAACCACAAGCCGGGGGG
SEQ ID NO: 8 (PpyLit amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R T A C V R F S H A R D P
I F G N Q I I P D T A I L S V V P F H H G F G M F
T T L G Y L I C G F R V V L M Y R F E E E L F L R
S L Q D Y K I Q S A L L V P T L F S F F A K S T L
I D K Y D L S N L H E I A S G G A P L S K E V G E
A V A K R F H L P G I R Q G Y G L T E T T S A I L
I T P E G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V P
P A E L E S V L L Q H P N I F D A G V A G V P D S
E A G E L P G A V V V M E K G K T M T E K E I V D
Y V N S Q V V N H K R L R G G V R F V D E V P K G
L T G K I D A K V I R E I L K K P Q A G G

In certain embodiments, the chimeric luciferase comprises PpyWT (SEQ ID NO:3 and SEQ ID NO:4) with one or more amino acid changes selected from the group consisting of Ile457Val, Ala482Gly, His489Lys, Ala503Asn and Lys543Gln. In an exemplified embodiment, the chimeric luciferase comprises PpyWt with the amino acid change Ile457Val/Ala482Gly/His489Lys/Ala503Asn/Lys543Gln. This may also be referred to as Ppy5.

In another exemplified embodiment, the chimeric luciferase comprises the nucleic acid sequence of SEQ ID NO:9 and the corresponding deduced amino acid sequence, SEQ ID NO:10, shown below.

SEQ ID NO: 9
(Ppy5 nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGGTATTGTTACAACACCCCAACATCTTCGACGC
GGGCGTGGCAGGTCTTCCCGACGATGACGCCGGTGAACTTCCGGGCGCCG
TGGTTGTTTTGGAGAAGGGAAAGACGATGACGGAAAAAGAGATCGTGGAT
TACGTAAACAGTCAAGTAACAACCGCGAAAAAGTTGCGCGGAGGAGTTGT
GTTTGTGGACGAAGTACCGAAAGGTCTTACCGGAAAACTCGACGCTAGAA
AAATCAGAGAGATCCTCATAAAGGCCCAGAAGGGCGGAAAGTCCAAATTG
SEQ ID NO: 10
(Ppy5 amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R T A C V R F S H A R D P
I F G N Q I I P D T A I L S V V P F H H G F G M F
T T L G Y L I C G F R V V L M Y R F E E E L F L R
S L Q D Y K I Q S A L L V P T L F S F F A K S T L
I D K Y D L S N L H E I A S G G A P L S K E V G E
A V A K R F H L P G I R Q G Y G L T E T T S A I L
I T P E G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S V L L Q H P N I F D A G V A G L P D D
D A G E L P G A V V V L E K G K T M T E K E I V D
Y V N S Q V T T A K K L R G G V V F V D E V P K G
L T G K L D A R K I R E I L I K A Q K G G K S K L

In other embodiments, the chimeric luciferase comprises the nucleic acid sequence of SEQ ID NO:21 and the amino acid sequence, SEQ ID NO:22, with amino acid sequence changes selected from one or more of the group consisting of Ile457Val, Ala482Gly, His489Lys, Ala503Asn, Lys543Gln and I351V. SEQ ID NO:21 and SEQ ID NO: 22 are shown below.

SEQ ID NO: 21
(Ppy WTTS cDNA sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCAATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACTAGTGCTATTCTG
ATTACACCCAAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGATATTGTTACAACACCCCAACATCTTCGACGC
GGGCGTGGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
SEQ ID NO: 22
(Ppy WTTS amino acid sequence)
MEDAKNIKKGPAPFYPLEDGTAGEQLHKAMKRYALVPGTIAFTDAHIEVN
ITYAEYFEMSVRLAEAMKRYGLNTNHRIVVCSENSLQFFMPVLGALFIGV
AVAPANDIYNERELLNSMNISQPTVVFVSKKGLQKILNVQKKLPIIQKII
IMDSKTDYQGFQSMYTFVTSHLPPGFNEYDFVPESFDRDKTIALIMNSSG
STGLPKGVALPHRALCVRFSHARDPIFGNQIAPDTAILSVVPFHHGFGMF
TTLGYLICGFRVVLMYRFEEELFLRSLQDYKIQSALLVPTLFSFLAKSTL
IDKYDLSNLHEIASGGAPLSKEVGEAVAKRFHLPGIRQGYGLTETTSAIL
ITPKGDDKPGAVGKVVPEPEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKSKL

In a further embodiment, the chimeric luciferase comprises the nucleic acid sequence of SEQ ID NO:21 and the amino acid sequence, SEQ ID NO:22, with the amino acid sequence change Ile457Val/Ala482Gly/His489Lys/Ala503Asn/Lys543Gln/I351V. This may also be referred to as PpyWT-TS5/I351V.

An exemplified chimeric luciferase comprises the nucleic acid sequence of SEQ ID NO:11 and the corresponding amino acid sequence, SEQ ID NO:12, shown below.

SEQ ID NO: 11
(Ppy WT-TS5/1351V nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCAATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACTAGCGCTATTCTG
GTAACACCCAAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGGTATTGTTACAACACCCCAACATCTTCGACGC
GGGCGTGGCAGGTCTTCCCGACGATGACGCCGGTGAACTTCCGGGCGCCG
TGGTTGTTTTGGAGAAGGGAAAGACGATGACGGAAAAAGAGATCGTGGAT
TACGTAAACAGTCAAGTAACAACCGCGAAAAAGTTGCGCGGAGGAGTTGT
GTTTGTGGACGAAGTACCGAAAGGTCTTACCGGAAAACTCGACGCTAGAA
AAATCAGAGAGATCCTCATAAAGGCCCAGAAGGGCGGAAAGTCCAAATTG
SEQ ID NO: 12
(Ppy WT-TS5/I351V amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R A L C V R F S H A R D P
I F G N Q I A P D T A I L S V V P F H H G F G M F
T T L G Y L I C G F R V V L M Y R F E E E L F L R
S L Q D Y K I Q S A L L V P T L F S F L A K S T L
I D K Y D L S N L H E I A S G G A P L S K E V G E
A V A K R F H L P G I R Q G Y G L T E T T S A I L
V T P K G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S V L L Q H P N I F D A G V A G L P D D
D A G E L P G A V V V L E K G K T M T E K E I V D
Y V N S Q V T T A K K L R G G V V F V D E V P K G
L T G K L D A R K I R E I L I K A Q K G G K S K L

In other embodiments, the chimeric luciferase comprises the nucleic acid sequence of SEQ ID NO:23 and the amino acid sequence, SEQ ID NO:24, with amino acid sequence changes selected from one or more of the group consisting of: Ile457Val, Ala482Gly, His489Lys, Ala503Asn, Lys543Gln and I351V. SEQ ID NO:23 and SEQ ID NO:24 are shown below.

SEQ ID NO: 23
(Ppy RETS cDNA sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCAATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
CTGCGTTACTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAAGGCTTCCATCTTC
CAGGGATACGCCAAGGATATGGGCTCACTGAGACTACTAGTGCTATTCTG
GTAACACCCATCGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCCATATTGTTACAACACCCCAACATCCGGGACGC
GGGCGTGGCAGGTCTTCCCGACGATGACGCCGGTGAACTTCCCGCCGCCG
TTGTTGTTTTGGAGCACGGAAAGACGATGACTGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
SEQ ID NO: 24
(Ppy RETS amino acid sequence)
MEDAKNIKKGPAPFYPLEDGTAGEQLHKAMKRYALVPGTIAFTDAHIEVN
ITYAEYFEMSVRLAEAMKRYGLNTNHRIVVCSENSLQEEMPVLGALFIGV
AVAPANDIYNERELLNSMNISQPTVVEVSKKGLQKILNVQKKLPIIQKII
IMDSKTDYQGFQSMYTEVTSHLPPGFNEYDFVPESFDRDKTIALIMNSSG
STGLPKGVALPHRALCVRFSHARDPIEGNQIAPDTAILSVVPFHHGEGME
TTLGYLICGERVVLMYRFEEELFLRSLQDYKIQTALLVPTLESFLAKSTL
IDKYDLSNLHEIASGGAPLSKEVGEAVAKRFHLPGIRQGYGLTETTSAIL
ITPKGDDKPGAVGKVVPEPEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKSKL

In a further embodiment, the chimeric luciferase comprises the nucleic acid sequence of SEQ ID NO:23 and the amino acid sequence, SEQ ID NO:24, with the amino acid sequence change Ile457Val/Ala482Gly/His489Lys/Ala503Asn/Lys543Gln/I351V. This may also be referred to as PpyRE-TS5/I351V.

An exemplified chimeric luciferase comprises the nucleic acid sequence of SEQ ID NO:13 and the corresponding amino acid sequence, SEQ ID NO:14, shown below.

SEQ ID NO: 13
(Ppy RE-TS5/I351V nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCAATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
CTGCGTTACTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACTAGCGCTATTCTG
GTAACACCCAAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGGTATTGTTACAACACCCCAACATCTTCGACGC
GGGCGTGGCAGGTCTTCCCGACGATGACGCCGGTGAACTTCCGGGCGCCG
TGGTTGTTTTGGAGAAGGGAAAGACGATGACGGAAAAAGAGATCGTGGAT
TACGTAAACAGTCAAGTAACAACCGCGAAAAAGTTGCGCGGAGGAGTTGT
GTTTGTGGACGAAGTACCGAAAGGTCTTACCGGAAAACTCGACGCTAGAA
AAATCAGAGAGATCCTCATAAAGGCCCAGAAGGGCGGAAAGTCCAAATTG
SEQ ID NO: 14
(Ppy RE-TS5/I351V amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R A L C V R F S H A R D P
I F G N Q I A P D T A I L S V V P F H H G F G M F
T T L G Y L I C G F R V V L M Y R F E E E L F L R
S L Q D Y K I Q T A L L V P T L F S F L A K S T L
I D K Y D L S N L H E I A S G G A P L S K E V G E
A V A K R F H L P G I R Q G Y G L T E T T S A I L
V T P K G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S V L L Q H P N I F D A G V A G L P D D
D A G E L P G A V V V L E K G K T M T E K E I V D
Y V N S Q V T T A K K L R G G V V F V D E V P K G
L T G K L D A R K I R E I L I K A Q K G G K S K L

In other embodiments, the chimeric luciferase comprises the nucleic acid sequence of SEQ ID NO:19 and the amino acid sequence, SEQ ID NO:20, with amino acid sequence changes selected from one or more of the group consisting of: Ile457Val, Arg465Phe, Ala482Gly, His489Lys, Ala503Asn and Lys543Gln. SEQ ID NO:19 and SEQ ID NO: 20 are shown below.

SEQ ID NO: 19
(Ppy RE9 cDNA sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCTATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
CTGCGTTACTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAAGGCTTCCATCTTC
CAGGGATACGCCAAGGATATGGGCTCACTGAGACTACTAGTGCTATTCTG
GTAACACCCATCGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCCATATTGTTACAACACCCCAACATCCGGGACGC
GGGCGTGGCAGGTCTTCCCGACGATGACGCCGGTGAACTTCCCGCCGCCG
TTGTTGTTTTGGAGCACGGAAAGACGATGACTGAAAAAGAGATCGTGGAT
TACGTCGCCAGTCAAGTAACAACCGCGAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
SEQ ID NO: 20
(Ppy RE9 amino acid sequence)
MEDAKNIKKGPAPFYPLEDGTAGEQLHKAMKRYALVPGTIAFTDAHIEVN
ITYAEYFEMSVRLAEAMKRYGLNTNHRIVVCSENSLQFFMPVLGALFIGV
AVAPANDIYNERELLNSMNISQPTVVEVSKKGLQKILNVQKKLPIIQKII
IMDSKTDYQGFQSMYTEVTSHLPPGFNEYDFVPESFDRDKTIALIMNSSG
STGLPKGVALPHRALCVRFSHARDPIEGNQIAPDTAILSVVPFHHGEGME
TTLGYLICGERVVLMYRPEEELFLRSLQDYKIQTALLVPTLESFLAKSTL
IDKYDLSNLHEIASGGAPLSKEVGEAVAKGFHLPGIRQGYGLTETTSAIL
VTPIGDDKPGAVGKVVPFFEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIRDAGVAGLPDDDAGELPAAVVVLEHGKTMTEKEIVD
YVASQVTTAKKLRGGVVFVDEVPKGLTGKLDARKIREILIKAKKGGKSKL

In a further embodiment, the chimeric luciferase comprises the nucleic acid sequence of SEQ ID NO:19 and the amino acid sequence, SEQ ID NO:20, with the amino acid sequence changes Ile457Val/Arg465Phe/Ala482Gly/His489Lys/Ala503Asn/Lys543Gln. This may also be referred to as PpyRE13.

An exemplified chimeric luciferase comprises the nucleic acid sequence of SEQ ID NO:15 and the corresponding amino acid sequence, SEQ ID NO:16, shown below.

SEQ ID NO: 15
(Ppy RE13 nucleic acid sequence)
ATGGAAGACGCCAAAAACATAAAGAAAGGCCCGGCGCCATTCTATCCTCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAATTCCTCTGGA
TCTACTGGGTTACCTAAGGGTGTGGCCCTTCCGCATAGAGCTCTCTGCGT
CAGATTCTCGCACGCCAGAGATCCAATATTTGGCAATCAAATCGCTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
CTGCGTTACTAGTACCAACCCTATTTTCATTCTTGGCCAAAAGTACTCTG
ATTGACAAATACGATTTATCTAATTTACACGAAATTGCTTCTGGGGGCGC
ACCTCTTTCGAAAGAAGTCGGGGAAGCGGTTGCAAAAGGCTTCCATCTTC
CAGGGATACGCCAAGGATATGGGCTCACTGAGACTACTAGTGCTATTCTG
GTAACACCCATCGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATATCAGGTGGCC
CCCGCTGAATTGGAATCGGTATTGTTACAACACCCCAACATCTTCGACGC
GGGCGTGGCAGGTCTTCCCGACGATGACGCCGGTGAACTTCCGGGCGCCG
TGGTTGTTTTGGAGAAGGGAAAGACGATGACGGAAAAAGAGATCGTGGAT
TACGTAAACAGTCAAGTAACAACCGCGAAAAAGTTGCGCGGAGGAGTTGT
GTTTGTGGACGAAGTACCGAAAGGTCTTACCGGAAAACTCGACGCTAGAA
AAATCAGAGAGATCCTCATAAAGGCCCAGAAGGGCGGAAAGTCCAAATTG
SEQ ID NO: 16
(Ppy RE13 amino acid sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R A L C V R F S H A R D P
I F G N Q I A P D T A I L S V V P F H H G F G M F
T T L G Y L I C G F R V V L M Y R F E E E L F L R
S L Q D Y K I Q T A L L V P T L F S F L A K S T L
I D K Y D L S N L H E I A S G G A P L S K E V G E
A V A K G F H L P G I R Q G Y G L T E T T S A I L
V T P I G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S V L L Q H P N I F D A G V A G L P D D
D A G E L P G A V V V L E K G K T M T E K E I V D
Y V N S Q V T T A K K L R G G V V F V D E V P K G
L T G K L D A R K I R E I L I K A Q K G G K S K L

In other embodiments, SEQ ID NOs 15 and 16/are codon optimized. In a related embodiment, an exemplified chimeric luciferase comprises the human codon optimized nucleic acid sequence of SEQ ID NO:17 and the corresponding deduced amino acid sequence, SEQ ID NO:18, shown below.

SEQ ID NO: 17
(Human codon optimized Ppy RE13 nucleic acid
sequence)
ATGGAGGACGCCAAGAACATCAAGAAGGGACCAGCCCCCTTCTACCCCCT
GGAGGACGGCACAGCCGGCGAGCAGCTGCACAAGGCCATGAAGCGGTACG
CCCTGGTGCCAGGCACCATCGCCTTCACCGACGCCCACATCGAGGTGAAC
ATCACCTACGCCGAGTACTTCGAGATGAGCGTGCGGCTGGCCGAGGCCAT
GAAGCGGTACGGCCTGAACACCAACCACCGGATCGTGGTGTGCAGCGAGA
ACAGCCTGCAGTTCTTCATGCCCGTGCTGGGAGCCCTGTTCATCGGCGTG
GCCGTGGCCCCAGCCAACGACATCTACAACGAGCGGGAGCTGCTGAACAG
CATGAACATCAGCCAGCCCACCGTGGTGTTCGTGAGCAAGAAGGGCCTGC
AGAAGATCCTGAATGTGCAGAAGAAGCTGCCCATCATCCAGAAGATCATC
ATCATGGACAGCAAGACCGATTACCAGGGCTTCCAGAGCATGTACACCTT
CGTGACCAGCCACCTGCCCCCAGGCTTCAACGAGTACGACTTCGTGCCCG
AGAGCTTCGACCGGGACAAGACCATCGCCCTGATCATGAACAGCAGCGGC
AGCACCGGCCTGCCCAAGGGCGTGGCCCTGCCCCACCGGGCCCTGTGCGT
GCGGTTCAGCCACGCCAGAGACCCCATCTTCGGCAACCAGATCGCCCCCG
ACACCGCCATCCTGAGCGTGGTGCCCTTCCACCACGGCTTCGGCATGTTC
ACCACCCTGGGCTACCTGATCTGCGGCTTCCGGGTGGTGCTGATGTACAG
GTTCGAGGAGGAGCTGTTCCTGCGGAGCCTGCAGGACTACAAGATCCAGA
CCGCCCTGCTGGTGCCCACCCTGTTCAGCTTCCTGGCCAAGAGCACCCTG
ATCGACAAGTACGACCTGAGCAACCTGCACGAGATCGCCTCTGGCGGAGC
CCCACTGAGCAAGGAGGTGGGCGAGGCCGTGGCCAAGGGCTTCCACCTGC
CAGGCATCCGGCAGGGCTACGGCCTGACCGAGACCACCAGCGCCATCCTG
GTGACCCCCATCGGCGACGACAAGCCCGGAGCCGTGGGCAAGGTGGTGCC
CTTCTTCGAGGCCAAGGTGGTGGACCTGGACACCGGCAAGACCCTGGGCG
TGAACCAGAGAGGCGAGCTGTGCGTGAGAGGCCCCATGATCATGAGCGGC
TACGTGAACAACCCCGAGGCCACCAACGCCCTGATCGACAAGGACGGCTG
GCTGCACAGCGGCGACATCGCCTACTGGGACGAGGACGAGCACTTCTTCA
TCGTGGACCGGCTGAAGAGCCTGATCAAGTATAAAGGCTATCAGGTCGCC
CCCGCCGAACTGGAAAGCGTCCTGCTGCAGCACCCTAACATCTTCGATGC
CGGAGTGGCTGGACTGCCAGACGATGACGCAGGAGAGCTGCCTGGAGCTG
TGGTCGTGCTGGAAAAGGGCAAAACTATGACCGAGAAGGAAATCGTCGAT
TACGTGAACAGCCAGGTGACCACAGCCAAGAAACTGCGAGGAGGAGTCGT
GTTCGTCGACGAGGTGCCCAAGGGACTGACAGGCAAACTGGACGCACGCA
AGATTAGGGAGATCCTGATTAAGGCACAGAAGGGGGGAAAGATT
SEQ ID NO: 18
(Human codon optimized Ppy RE13 amino acid
sequence)
M E D A K N I K K G P A P F Y P L E D G T A G E Q
L H K A M K R Y A L V P G T I A F T D A H I E V N
I T Y A E Y F E M S V R L A E A M K R Y G L N T N
H R I V V C S E N S L Q F F M P V L G A L F I G V
A V A P A N D I Y N E R E L L N S M N I S Q P T V
V F V S K K G L Q K I L N V Q K K L P I I Q K I I
I M D S K T D Y Q G F Q S M Y T F V T S H L P P G
F N E Y D F V P E S F D R D K T I A L I M N S S G
S T G L P K G V A L P H R A L C V R F S H A R D P
I F G N Q I A P D T A I L S V V P F H H G F G M F
T T L G Y L I C G F R V V L M Y R F E E E L F L R
S L Q D Y K I Q T A L L V P T L F S F L A K S T L
I D K Y D L S N L H E I A S G G A P L S K E V G E
A V A K G F H L P G I R Q G Y G L T E T T S A I L
V T P I G D D K P G A V G K V V P F F E A K V V D
L D T G K T L G V N Q R G E L C V R G P M I M S G
Y V N N P E A T N A L I D K D G W L H S G D I A Y
W D E D E H F F I V D R L K S L I K Y K G Y Q V A
P A E L E S V L L Q H P N I F D A G V A G L P D D
D A G E L P G A V V V L E K G K T M T E K E I V D
Y V N S Q V T T A K K L R G G V V F V D E V P K G
L T G K L D A R K I R E I L I K A Q K G G K I

### Methods of Use

Generally, light intensity of firefly bioluminescence is correlated to the chemical concentrations of the reaction components. When configured properly, the light intensity can be used to associate an observable parameter with a molecular process. Most commonly this is done by holding the concentrations of all components in the luminescent reaction constant, except for one that is allowed to vary in correlation with the process of interest. Depending on the assay design, the variable component may be ATP, luciferin, or the enzyme itself.

The luminescence generated by a luciferase reaction is typically detected with a luminometer, although other detection means maybe used. The presence of light greater than background level indicates the presence of ATP in the sample. The background level of luminescence is typically measured in the same matrix in which the sample exists, but in the absence of the sample. Suitable control reactions are readily designed by one of skill in the art. Preferred luciferases used in the compositions and methods of the invention have enhanced thermostability properties and/or show increased resistance to color shifting compared to wild type.

The chimeric firefly luciferases described herein may be used in methods for determining transcriptional activity, in in vivo imaging of for determining ATP utilizing or generating enzyme activity. Further, in other embodiments, the effect of one or more compounds on kinase enzyme activity, protease activity, P-450 enzyme activity, and ATP utilizing or generating enzyme activity contained in a sample can be determined using the chimeric luciferases described herein.

For the introduction of a peptide of the invention, respectively the nucleic acid encoding it, into a suitable host cell and its expression it can be advantageous if the nucleic acid is integrated in an expression vector. Cloning techniques to introduce a nucleic acid into a suitable expression vector for subsequent transformation of a cell and subsequent selection of the transformed cell are well known in the art (see for example Sambrook et al. (1989), Molecular cloning: A laboratory Manual, Cold Spring Harbour Laboratory, incorporated by reference in its entirety herein).

Accordingly, the invention features an expression vector comprising a nucleic acid sequence encoding any of the chimeric firefly luciferases described herein. In further embodiments of the present invention there is thus provided a vector, preferably an expression vector, comprising a nucleic acid encoding a peptide of the invention. Suitable vectors are known in the art.

The expression vector can be a eukaryotic expression vector, or a retroviral, lentiviral, adenoviral or adenoviral associated vector, a expression vector, bacteriophage, or any other vector typically used in the biotechnology field. The vectors may contain one or more selection markers, such as an antibiotic resistance marker, for example. The nucleic acid encoding the chimeric luciferase peptide of the invention may be operatively linked to one or more regulatory elements which modulate the transcription and the synthesis of a translatable mRNA in pro- or eukaryotic cells. Such regulatory elements may be promoters, enhancers or transcription termination signals, but can also comprise introns or similar elements, for example those which promote or contribute to the stability and the amplification of the vector, the selection for successful delivery and/or the integration into the host's genome, like regions that promote homologous recombination at a desired site in the genome.

Nucleic acid molecules of the invention may be inserted into the vectors described herein in a sense orientation, or in an anti-sense orientation in order to provide for the production of anti-sense RNA.

The vectors described herein may be transformed into a host cell to allow expression of a peptide in accordance with the invention. The cell may be part of a tissue or an organism. The vector in which the above-described nucleic acid has been inserted can be used to obtain a transformant by transforming a well-known host such as Escherichia coli, yeast, Bacillus subtillis, leishmania, an insect cell, or a mammalian cell therewith by well-known methods. In the case of carrying out the transformation, a more preferable system is exemplified by the method for integrating the gene in the chromosome, in view of achieving stability of the gene. However, an autonomous replication system using a expression vector can be conveniently used. Introduction of the DNA vector into the host cell can be carried out by standard methods such that described in “Molecular Cloning: A Laboratory Manual” (ed. by Sambrook et al., Cold Spring Harbor Laboratory Press, Cold Spring Harbor, N.Y., 1989, incorporated by reference in its entirety herein.) In particular, calcium phosphate transfection, DEAE-dextran-mediated transfection, microinjection, cation lipid-mediated transfection, electroporation, transduction, scrape loading, ballistic introduction and infection can be employed.

In a further aspect there is provided a host cell transformed or transfected with an expression vector of the invention.

### Codon Optimization

In certain preferred embodiments, the chimeric firefly luciferase is expressed from a mammalian codon optimized gene. A “codon-optimized gene” is a gene having its frequency of codon usage designed to mimic the frequency of preferred codon usage of the host cell. As is well known in the art, this can be a useful means to further optimize the expression of the enzyme in the alternate host, since use of host-preferred codons can substantially enhance the expression of the foreign gene encoding the polypeptide. In general, host-preferred codons can be determined within a particular host species of interest by examining codon usage in directing protein synthesis (preferably those expressed in the largest amount), and determining which codons are used with highest frequency. Then, the coding sequence for a polypeptide of interest having e.g., elongase activity can be synthesized in whole or in part using the codons preferred in the host species. All (or portions) of the DNA also can be synthesized to remove any destabilizing sequences or regions of secondary structure that would be present in the transcribed mRNA. All (or portions) of the DNA also can be synthesized to alter the base composition to one more preferable in the desired host cell.

In certain exemplary embodiments, the chimeric luciferase comprises the human codon optimized nucleic acid sequence of SEQ ID NO:17 and the corresponding deduced amino acid sequence, SEQ ID NO:18.

### Detection of Transcriptional Activity

In biological research, luciferase commonly is used as a reporter to assess the transcriptional activity in cells that are transfected with a genetic construct containing the luciferase gene under the control of a promoter of interest.

For example, in high throughput applications, luciferase has been best known as a genetic reporter. Many cellular events of relevance to drug discovery can be associated with regulation of gene transcription. By coupling the operative regulatory elements to expression of a luciferase gene, typically by placing the regulatory element just upstream of a gene encoding luciferase, the cellular event can be readily detected by a luminescent signal.

In certain aspects the present invention features methods of detection of transcriptional activity in a cell comprising introducing a expression vector comprising the a nucleic acid sequence encoding a chimeric firefly luciferase as described herein, preferably with a promoter sequence, into a cell, wherein the expression vector comprises a promoter of interest, and detecting the light emission, wherein the detection of light emission indicates transcriptional activity.

### In Vivo Imaging

Whole animal imaging (in vivo or ex vivo) can be used for studying cell populations in live animals, such as mice. Different types of cells (e.g. bone marrow stem cells, T-cells) can be engineered to express a luciferase allowing their non-invasive visualization inside a live animal using a sensitive charge-couple device camera (CCD camera). For example, this technique has been used to follow tumorigenesis and response of tumors to treatment in animal models.

Accordingly, in other certain aspects the present invention features a method for in vivo imaging comprising introducing the chimeric luciferase of any one of the above claims into a cell of a living animal and detecting the light emission.

### ATP Detection

ATP assays depend on reporter molecules or labels to qualitatively or quantitatively monitor ATP levels. Reporter molecules or labels in such assay systems have included radioactive isotopes, fluorescent agents, and enzymes, including light-generating enzymes such as luciferase. Desirable characteristics of any reporter molecule systems include safe, quick and reliable application and detection. Luminescent systems are among the most desirable since they are exceptionally safe and sensitive.

When luciferase is combined with a sample for the purpose of detecting ATP, it is typically desirable to inhibit ATPases endogenous to the sample as well as enzymes that generate ATP, thus assuring that the ATP detected corresponds to the actual amount of ATP in a sample at a desired time. Many ATPase inhibitors are known, including detergents, especially detergents that are positively charged. However, most ATPase inhibitors are effective in not only eliminating ATPase function endogenous to the sample (e.g., a cell or cell population), but also ATPases that may be used as the reporter molecule, such as luciferase. Additionally, to counter ATP production, inhibitors of enzymes that phosphorylate, such as kinases, are desirable.

Examples of ATPase inhibitors include detergents, preferably detergents with charged groups such as cationic detergents [e.g., DTAB (dodecyltrimethylammonium bromide), CTAB (cetyltrimethylammonium) and BDDABr (benzyldimethyldodecylammonium bromide)], anionic detergents (e.g., SDS and deoxycholate), and zwitterionic detergents (e.g., sulfobetaine 3-10). To facilitate the method, a substrate for the luciferase, such as luciferin, may be included in the reagent composition. Other embodiments of the reagent composition further comprise a compound, such as NaF, that prevents an increase in ATP levels in the sample over time. Other compounds that prevent an increase in ATP levels in the sample include vanadate and paranitrophenylphosphate. Still other embodiments of the reagent composition further comprise a buffer and magnesium. One of skill in the art knows that other cations, such as manganese and calcium, may be suitable substitutes for magnesium.

Among the assay systems in which bioluminescence has been employed to monitor or measure ATP are those in which the activity of an ATP-dependent bioluminescent enzyme, e.g. a beetle luciferase, is exploited. There are multiple variations of cellular ATP detection methods currently used. Some such methods first lyse the cells and inactivate the ATPase activity endogenous to the sample (e.g., by increasing sample pH), then neutralize the ATPase inhibitor, thereby converting the environment of the sample to one favorable to luciferase activity prior to adding the luciferase and detecting luminescence. Other such methods combine the neutralization of the ATPase inhibitor with the addition of luciferase.

In certain aspects, the invention is drawn to methods, compositions and kits that are used to detect and quantify ATP levels in a sample. The method comprises adding to a sample a composition comprising a chimeric firefly luciferase enzyme and an ATPase inhibitor, and detecting luminescence produced in the sample by the conversion of a substrate into a luminescing compound by luciferase.

The luminescence generated by a luciferase reaction is typically detected with a luminometer although other detection means may be used. The presence of light greater than background level indicates the presence of ATP in the sample. The background level of luminescence is typically measured in the same matrix in which the sample exists, but in the absence of the sample. Suitable control reactions are readily designed by one of skill in the art. Preferred luciferases used in the compositions and methods of the invention generate a stable signal, i.e., they yield enhanced duration of luminescence in a luciferase reaction defined as less than 50% loss of luminescence per hour relative to the luminescence at the time the luciferase reaction was initiated. Preferred luciferases of the invention allow for multiple analyses of a sample over time or analysis of many samples over time, one hour after the luciferase is combined with the ATPase inhibitor, more preferably two hours and most preferably four hours or more. The luciferases used in the compositions and methods of the invention have enhanced thermostability and/or color shifting properties.

Quantifying the amount of emitted light also quantifies the amount of ATP in a sample, and thereby the quantity of living cells. Quantitative ATP values are realized, for example, when the quantity of light emitted from a test sample is compared to the quantity of light emitted from a control sample or to a standard curve determined by using known amounts of ATP and the same luciferase, substrate, and reaction conditions (i.e. temperature, pH, etc.). It is understood that quantification involves subtraction of background values. Qualitative ATP values are realized when the luminescence emitted from one sample is compared to the luminescence emitted from another sample without a need to know the absolute amount of ATP present in the samples, e.g., a comparison of samples in the presence or absence of a test compound. Many such experiments can readily be designed by one of ordinary skill in the art.

### Kits

The invention also comprises test kits for carrying out the assay methods of the invention. Such kits comprise, in one or more containers or packages, quantities of various compositions essential for carrying out the assays in accordance with the invention.

Thus, in certain embodiments, the kits include the chimeric luciferase described in any one of the aspects of the invention, e.g. a chimeric firefly luciferase comprising an N-terminal domain from a first firefly luciferase and a C-terminal amino acid domain from a second firefly luciferase.

For example, in kits for assaying for luciferase, there will be a chimeric firefly luciferase as described in any one of the aspects herein, and further comprising magnesium ion, ATP and luciferin, well known to be essential for the reaction. As indicated, the various components can be combined, e.g. in solution or a lyophilized mixture, in a single container or in various combinations (including individually) in a plurality of containers. In a preferred kit for assaying for luciferase in cells, in which the luciferase is expressed, there will also be included a solution (or the components for preparing a solution) useful for lysing the cells while preserving (against the action of various enzymes released during lysis) luciferase that might be in the cells in an active form, or a form which can be made active.

The present invention also includes kits for detecting or assaying for the amount of ATP in a sample.

The test kits of the invention can also include, various controls and standards well-known to one skilled in the art, for example solutions of known luciferase or ATP concentrations, including no luciferase or ATP (negative control), to ensure the reliability and accuracy of the assays carried out using the kits, and to permit quantitative analyses of samples for the analytes (e.g., luciferase, ATP) of the kits.

The following examples illustrate, but do not limit, the methods and compositions of the present invention and their embodiments.

### EXAMPLES

The Experiments described herein were carried out with, but not limited to, the following materials and methods.

### Materials and Methods

Abbreviations used. CB, 20 mM Tris-HCl (pH 7.4 at 4° C.) containing 150 mM NaCl, 1 mM EDTA and 1 mM DTT; CBA, CB containing 0.8 M ammonium sulfate and 2% glycerol; CCD, charge-coupled device; HCO, human codon optimized; LC/ESMS, tandem HPLC-electrospray ionization mass spectrometry; L, dehydroluciferin; L-AMP, dehydroluciferyl-AMP; LH₂, D-firefly luciferin; LitWT, recombinant Luciola italica luciferase (UniProtKB: Q1AG35) containing the changes Lys547Gly/Met548Gly and the additional N-terminal peptide GPLGS (SEQ ID NO: 25)-; LitPpy, chimeric protein comprised of (P. pyralis numbering) LitWT residues 1-439 and PpyWT residues 440-550 containing the additional N-terminal peptide GPLGS (SEQ ID NO: 25)-; 6×His-PpyWT (‘6×His’ disclosed as SEQ ID NO: 26), PpyWT with the GPLGS (SEQ ID NO: 25) N-terminal peptide replaced with MRGSHHHHHHGS (SEQ ID NO: 27); Native Ppy, native firefly luciferase from Photinus pyralis; PBS, phosphate buffer saline, pH 7.3; PpyWT, recombinant Photinus pyralis luciferase (UniProtKB: P08659) containing the additional N-terminal peptide GPLGS (SEQ ID NO: 25)-; PpyLit, chimeric protein comprised of (P. pyralis numbering) PpyWT residues 1-439 and LitWT residues 440-550 containing the additional N-terminal peptide GPLGS (SEQ ID NO: 25)-; Ppy5, PpyWT containing the following amino acid changes: Ile457Val, Ala482Gly, His489Lys, Ala503Asn and Lys543Gln; Ppy14, PpyWT containing the following amino acid changes: Ala450Pro, Ile457Val, Leu472Val, Asp475Ser, Asp476Glu, Ala482Gly, Leu487Met, His489Lys, Ala503Asn, Thr507Val, Thr508Asn, Ala509His, Lys511Arg, Val517Arg and Ppy19, Ppy14 containing the following amino acid changes: Leu530Ile, Lys534Val, Ile540Lys, Ala542Pro and Lys543Gln; Ppy RE13, Ppy RE9 (Anal. Biochem. 396, 290-297) containing the following amino acid changes: Ile457Val, Arg465Phe, Ala482Gly, His489Lys, Ala503Asn and Lys543Gln; Ppy RE-TS5, Ppy RE-TS5/I351V, PpyRE-TS (Anal. Biochem. 361, 253-262) containing the following amino acid changes: Ile457Val, Ala482Gly, His489Lys, Ala503Asn, Lys543Gln and Ile351Val; and Ppy WT-TS5, PpyWT-TS/I351V, PpyWT-TS (Anal. Biochem. 361, 253-262) containing the following amino acid changes: Ile457Val, Ala482Gly, His489Lys, Ala503Asn, Lys543Gln and Ile351Val.

Materials.

The following materials were obtained from the sources indicated: Mg-ATP (bacterial source) and native firefly luciferase from Photinus pyralis (Native Ppy) from Sigma-Aldrich (St. Louis, Mo.); restriction endonucleases and DNA ligase from New England Biolabs (Beverly, Mass.); mutagenic oligonucleotides from Integrated DNA technologies (Coralville, Iowa); Glutathione Sepharose 4B media and the pGEX-6P-2 expression vector from GE Healthcare (Piscataway, N.J.); pQE-30 expression vector, Ni-NTA agarose and QIAquick Gel Extraction kit from Qiagen (Valencia, Calif.) and the QuikChange® Lightning Site-Directed Mutagenesis kit from Stratagene (La Jolla, Calif.). The human codon optimized Ppy5 cDNA encoding residues 442-548 flanked by a 5′ PacI site and a 3′ NotI site was synthesized by GenScript (Piscataway, N.J.). Firefly luciferin (LH₂)¹ was a generous gift from Promega (Madison, Wis.). LH₂-AMP was prepared and purified as described previously (Branchini 2001). Native Ppy was resuspended in 100 mM sodium phosphate buffer, pH 7.8, containing 1 mM EDTA and dialyzed into 20 mM Tris-HCl (pH 7.4 at 4° C.) containing 150 mM NaCl, 1 mM EDTA and 1 mM DTT (CB). Solutions of the enzyme were stored at 4° C. following the addition of ammonium sulfate (0.8 M final) and glycerol (2% final). The found molecular masses (Da) of the following proteins were within the allowable experimental error (0.01%) of the calculated values (in parenthesis): PpyWT, 61161 (61157); 6×His-PpyWT (‘6×His’ disclosed as SEQ ID NO: 26), 62142 (62144); LitWT, 60764 (60766); LitPpy, 61094 (61095); PpyLit, 60812 (60811); Ppy5, 61159 (61163); Ppy14, 61337 (61341); Ppy19, 61347 (61353); PpyLit F465R, 60819 (60820); PpyLit I232A/E354K, 60770 (60768); PpyLit I351V/E354K, 60795 (60796); PpyLit I232A/I351V/E354K, 60750 (60754); PpyLit I351V/E354K/F465R, 60793 (60787); Ppy GR-TSLit, 60712 (60714); Ppy WT-TS5/I351V, 61084 (61086); Ppy S284T, 61165 (61171); PpyLit S284T, 60825 (60825); PpyLit S284T/F465R, 60836 (60834); PpyLit S284T/I351V/E354K, 60813 (60810); PpyLit S284T/I351V/E354K/F465R, 60814 (60819); PpyLit I232A/S284T/I351V/E354K/F465R, 60771 (60777); Ppy RE-TS5/I351V, 61094 (61098); Ppy RE-TSLit, 60740 (60746); Ppy RE13, 60982 (60985) and human codon optimized (HCO) Ppy RE 13, 60764 (60768). Ppy WT-TS, Ppy GR-TS and Ppy RE-TS were purified and characterized as described previously (Branchini et al. 2007 Anal Biochem. 361, 253-262).

General Methods.

Protein concentrations were determined with the Bio-Rad Protein Assay system using BSA as the standard. Site-directed mutagenesis was performed with the QuikChange® Lightning Site-Directed Mutagenesis kit from Stratagene (La Jolla, Calif.). DNA sequencing to verify all mutations and ligations was performed at the W. M. Keck Biotechnology Laboratory at Yale University.

Construction of LitPpy, PpyLit, Ppy14, Ppy GR-TSLit, Ppy RE-TSLit and 6×his-PpyWT (‘6×his’ Disclosed as SEQ ID NO: 26).

The pGEX-6P-2 expression plasmids containing the cDNA encoding LitPpy, Ppy GR-TSLit, Ppy RE-TSLit and PpyLit were generated as follows. The cDNA encoding the peroxisomal targeting signal (⁵⁴⁶AlaLysMet⁵⁴⁸) at the carboxyl terminus of Luciola italica luciferase (Branchini 2006) was mutated to ⁵⁴⁶AlaGlyGly⁵⁴⁸ using the following primer and its respective reverse compliment: 5′-AG AAA CCA CAA GCC GGG GGG TAA ATC GGT CAA AAT G-3′ (SEQ ID NO: 28) (the mutated codons are in bold). The encoded protein is referred to as LitWT. A PacI restriction site was introduced into the LitWT cDNA using the following primer and its respective reverse compliment: 5′-GTA GAT CGT TTG AAA TCA TTA ATT AAA TAC AAG GGG TAC CAG G-3′ (SEQ ID NO: 29) (underline represents the silent change to introduce the PacI site). Next, the pGEX-6P-2 plasmids containing LitWT (2), Ppy GR-TS (Anal. Biochem. 361, 253-262), Ppy RE-TS (Anal. Biochem. 361, 253-262) and PpyWT (3) were digested with PacI and XhoI generating two cDNA fragments for each construct encoding the N-terminal and C-terminal domains for each luciferase. The eight fragments were purified from an agarose gel with the QIAquick Gel Extraction kit and then ligated to create constructs in pGEX-6P-2 encoding the LitPpy, Ppy GR-TSLit, Ppy RE-TSLit and PpyLit chimeric proteins.

To construct the Ppy14 expression vector it was necessary to put AgeI restriction sites into the LitWT and PpyWT cDNA using the following primers and their respective reverse compliments: PpyWT, 5′-GAC GAA GTA CCG AAA GGT CTT ACC GGT AAA CTC GAC GCA AGA AAA ATC AG-3′ (SEQ ID NO: 30) and LitWT, 5′-GTG GAT GAA GTA CCT AAA GGT CTA ACC GGT AAA ATT GAT GCT AAA G-3′ (SEQ ID NO: 31) (underline represents the silent change to introduce the AgeI site). After digesting both expression vectors with AgeI and Pad, the PpyWT expression vector, which no longer contained the cDNA encoding residues 442-526, and the LitWT cDNA encoding residues 444-528, were purified from an agarose gel with the QIAquick Gel Extraction kit, and then ligated to create the expression vector encoding Ppy14.

To construct the expression vector encoding 6×His-Ppy WT (‘6×His’ disclosed as SEQ ID NO: 26), the pQE-30 expression vector was first modified to enable the insertion of the PpyWT cDNA from the pGEX-6P-2 vector where it is flanked by BamHI and XhoI sites. The existing XhoI site in the pQE-30 expression vector was removed and a new XhoI site was created in the multiple cloning site downstream of the BamHI site using the following primers and their respective reverse complements: 5′-ACG AGG CCC TTT CGT CTT CAC CTG GAG AAA TCA TAA AAA-3′ (SEQ ID NO: 32) and 5′-CAC GGA TCC GCA TGC GAG CTC GAG ACC CCG GGT CGA CCT-3′ (SEQ ID NO: 33), respectively. The gene encoding PpyWT was isolated from the pGEX-6p-2 expression vector by digestion with BamHI and XhoI, purified from an agarose gel with the QIAquick Gel Extraction kit and, ligated into the modified pQE-30 vector, which had been digested with the same restriction enzymes.

Site-Directed Mutagenesis.

Starting with the PpyLit cDNA in the pGEX-6P-2 expression plasmid, the QuikChange® Lightning Site-Directed Mutagenesis kit and the primers listed in Table 1 were used to generate the following mutations: F465R, I232A/E354K, I351V/E354K, I232A/I351V/E354K, I351V/E354K/F465R, S284T, S284T/F465R S284T/I351V/E354K, I232A/S284T/I351V/E354I, S284T/I351V/E354K/F465R, I232A/S284T/I351V/E354K/F465R. To make Ppy19, the L530I, K534V, 1540K, A542P and K543Q mutations were introduced into the Ppy14 cDNA using the primers listed in Table 1. Also, PpyWT D436G, PpyWT S284T and Ppy5 were made by introducing the required point mutations into the PpyWT cDNA with the indicated primers (Table 1). I351V was introduced into Ppy WT-TS5 and Ppy RE-TS5 using the I351V primer found in Table 1, below. Table 1 discloses SEQ ID NOS 34-48, respectively, in order of appearance.

TABLE 1
Primers used to create mutations in the cDNA encoding PpyWT, PpyLit or Ppy14.

| | | Screening |
|---|---|---|
| Mutation(s) | | Endo- |
| Introduced | Primers^{a} | nuclease |
| I232A | 5′-GCC AGA GAT CCA ATA TTT GGC AAT CAA ATC GCT CCG GAT ACT GC-3′ | SspI |
| S284T | 5′-CTT CAG GAT TAC AAA ATT CAA ACT GCG TTA CTA GTA CCA ACC-3′ | SpeI |
| I351V^{b} | 5′-GGG CTC ACT GAG ACT ACT AGC GCT ATT CTG GTA ACA CCC AAG GGG-3′ | SpeI |
| E354I | 5′-GAG ACT ACT AGT GCT ATT CTG GTA ACA CCC ATC GGG GAT GAT AAA C-3′ | SpeI |
| E354K | 5′-CT GAG ACT ACT AGT GCT ATT CTG ATT ACA CCC AAG GGG GAT GAT A-3′ | SpeI |
| D436G | 5′-GAA CAC TTC TTC ATA GTT GGC CGC TTG AAG TCT TTG ATT AAA TAC AAA G-3′ | PacI |
| I457V | 5′-AAA GGA TAT CAG GTG GCC CCC GCT GAA TTG GAA TCG GTA TTG TTA CAA CAC | none |
| | CCC-3′ | |
| F465R | 5′-CAT CCA AAT ATC CGT GAT GCT GGT GTG GCT-3′ | none |
| A482G/L487M/H489K | 5′-GCC GGT GAA CTT CCG GGC GCC GTT GTT GTT ATG GAG AAG GGA AAG ACG | EagI |
| | ATG ACG-3′ | |
| M487L^{c} | 5′-CTT CCG GGC GCC GTG GTT GTT TTG GAG AAG GGA AAG ACG-3′ | BtgI |
| L530I/K534V | 5′-CCG AAA GGT CTC ACC GGA AAA ATC GAC GCA AGA GTA ATC AGA GAG ATC | BsaI |
| | CTC-3′ | |
| A503N | 5′-GAG ATC GTG GAT TAC GTA AAC AGT CAA GTA ACA ACC GCG AAA AAG TTG-3′ | SnaBI |
| I540K/A542P^{d} | 5′-AAA ATC GAC GCT AGA GTA ATC AGA GAG ATC CTC AAA AAG CCA AAG AAG | BfaI |
| | GGC GG-3′ | |
| K543Q^{e} | 5′-AAA CTC GAC GCT AGA AAA ATC AGA GAG ATC CTC ATA AAG GCC CAG AAG | BfaI |
| | GGC GG-3′ | |
| K543Q^{f} | 5′-AAA ATC GAC GCA AGA GTA ATC AGA GAG ATC CTC AAA AAG CCA CAG AAG | BfaI |
| | GGC GG-3′ | |

^{a}Bold represents the mutated codon and underline represents silent changes to create or remove a unique screening endonuclease site.
^{b}Primer also contains the E354K change because this mutation was introduced earlier in the construction of PpyLit I351V/E354K. PpyLit I232A/I351V/E354K, PpyLit I351V/E354K/F465R and PpyLit S284T/I351V/E354K.
^{c}Primer also contains A482G/H489K and was used in the process of making Ppy5.
^{d}Primer also contains the L530I and K534V previously introduced in the process of making Ppy19.
^{e}Primers used to create Ppy5.
^{f}Primer also contains the L530I, K534V, I540K and A542P changes already present while making Ppy19.

Construction of Ppy WT-TS5, Ppy WT-TS5/I351V, Ppy RE-TS5, Ppy RE-TS5/I351V, Ppy RE13, and HCO Ppy RE13.

The pGEX-6P-2 expression plasmids containing the cDNA encoding Ppy WT-TS5, Ppy RE-TS5 and Ppy RE13 were generated as follows. The pGEX-6P-2 plasmids containing Ppy WT-TS (Anal. Biochem. 361, 253-262), Ppy RE-TS (Anal. Biochem. 361, 253-262), Ppy RE9 (Anal. Biochem. 396, 290-297) and Ppy5 were digested with PacI and XhoI generating two cDNA fragments for each construct encoding the N-terminal (residues 1-441) and C-terminal domains for each luciferase. N-terminal domain fragments for Ppy WT-TS, Ppy RE-TS and Ppy RE9 and the C-terminal domain fragment for Ppy 5 (residues 442-550 and does not contain the F465R mutation) were purified from an agarose gel with the QIAquick Gel Extraction kit and then ligated to create constructs in pGEX-6P-2 encoding the Ppy WT-TS5, Ppy RE-TS5 and Ppy RE13 proteins. I351V was introduced into Ppy WT-TS5 and Ppy RE-TS5 using the I351V primer found in Table 1.

To construct the HCO Ppy RE13 expression vector it was necessary to put the PacI restriction site into the HCO Ppy RE9 cDNA (GenBank accession number GQ404466) using the following primer and its respective reverse compliments: 5′-CGG CTG AAG AGC TTA ATT AAA TAC AAG GGC TAC CAG GTG-3′ (SEQ ID NO: 49) (underline represents the silent change to introduce the PacI site). The HCO Ppy5 cDNA encoding residues 442-548 flanked by a 5′ PacI site and a 3′ NotI site was synthesized by GenScript (Piscataway, N.J.) and provided in the puc57 vector. After digesting both vectors with PacI and NotI, the HCO Ppy RE9 expression vector, which no longer contained the cDNA encoding residues 442-548 and the Ppy5 cDNA encoding residues 442-548 were purified from an agarose gel with the QIAquick Gel Extraction kit and then ligated to create the expression vector encoding HCO Ppy RE13. The PacI site was then removed to restore the human optimization using the following primer and its respective reverse compliment: 5′-GTG GAC CGG CTG AAG AGC CTG ATC AAG TAT AAA GGC TAT CAG-3′ (SEQ ID NO: 50) (underline represents the silent change to remove the PacI site).

Protein Expression and Purification.

Luciferases in the pGex-6P-2 expression vector were expressed in E. coli strain BL21 as GST-fusion proteins. Cultures (0.25 L) were grown with shaking at 320 rpm in 1 L flasks at 37° C. in Luria broth supplemented with 100 μg/mL ampicillin until mid log phase (A₆₀₀=0.5-0.7), moved to a 22° C. incubator, allowed to equilibrate for 10 min, induced with 0.1 mM IPTG, and incubated at 22° C. for 18-20 h. The cells were harvested by centrifugation at 4° C. and then kept at −80° C. for 15 min. Cell pellets were resuspended in 25 mL phosphate buffered saline, pH 7.3 (PBS) containing 0.1 mM phenylmethylsulfonyl fluoride and 0.5 mM DTT. After the addition of 2.5 mL lysozyme solution (10 mg/mL in PBS), the cells were lysed by sonication and treated with DNase (5 μg/mL) and RNase (10 μg/mL) for 5 min on ice. Triton X-100 was added to the lysates (1% final volume) and the whole-cell extracts were isolated by centrifugation at 20,000×g for 1 h. Proteins were further purified using Glutathione Sepharose® 4B affinity chromatography according to the manufacturer's instructions. During the purification, luciferases were released from GST-fusion proteins by incubation with PreScission protease in CB for 18-20 h at 4° C. with gentle mixing. Proteins were eluted with CB (yields of ˜5 mg/0.25 L culture) and stored at 4° C. in CBA (Branchini 2007a).

6×His-PpyWT (′6×His' disclosed as SEQ ID NO: 26) was expressed in E. coli BL21 (pREP4) cells. A culture (0.25 L) was grown with shaking at 320 rpm in a 1 L flask at 37° C. in Luria broth supplemented with 100 μg/mL ampicillin and 25 μg/mL kanamycin until mid log phase (A_{600 nm}˜0.6), moved to a 22° C. incubator, allowed to equilibrate for 10 min, induced with 0.1 mM IPTG and incubated at 22° C. for 18 h. Cells were harvested by centrifugation at 4° C. and then frozen at −80° C. for 15 min. The cell pellet was resuspended in 25 mL of PBS containing 0.1 mM phenylmethylsulfonyl fluoride and 5 mM imidazole. After the addition of 2.5 mL lysozyme (10 mg/mL in PBS), the cells were lysed by sonication and treated with DNase (5 μg/mL) and RNase (10 μg/mL) for 5 min on ice. Triton X-100 was added (1% final volume) and the whole-cell extracts were isolated by centrifugation at 20,000×g for 45 min. 6×His-PpyWT (‘6×His’ disclosed as SEQ ID NO: 26) was purified using Ni-NTA agarose (Qiagen) affinity chromatography according to the manufacturer's instructions. Fractions eluted with 250-500 mM imidazole were pooled (2 mL) and dialyzed (2 changes, 1 L each) against CB and stored at 4° C. in CBA.

Determination of Protein Masses.

Mass spectral analyses were performed by tandem HPLC-electrospray ionization mass spectrometry (LC/ESIMS) using a ThermoFinnigan Surveyor HPLC system and a ThermoFinnigan LCQ Advantage mass spectrometer. The conditions for protein mass determinations were: column, Jupiter 5 μm C4 300 Å (50×1.00 mm); wavelength, 270 nm; mobile phase, 95:5 water (0.1% TFA):acetonitrile (0.1% TFA), gradient after 5 min to 5:95 water (0.1% TFA):acetonitrile (0.1% TFA) over 5 min; flow rate, 0.05 mL/min; MS mode, ES+; scan range, m/z=200-2000; scan time, 0.2 s. The electrospray source of the MS was operated with a capillary voltage of 37 V, and source voltage of 3.5 kV. Total mass spectra for protein samples were reconstructed from the ion series using Bioworks Browser 3.0 with BIOMASS deconvolution (Branchini 2011).

Bioluminescence Specific Activities.

Bioluminescence specific activity assays were performed with a custom-built luminometer assembly containing a Hamamatsu R928 PMT and a C6271 HV power supply socket assembly (Branchini 2007b). The instrument consists of a custom-built aluminum box fitted with an Aminco Chem Glow II sample compartment into which the side-reading PMT was fixed in the central area. The device accommodates 8×50 mm polypropylene tubes from Evergreen Scientific (Los Angeles, Calif.). The socket assembly is powered by a constant 12 V DC (ELPAC Power Systems model FW1812) and the high voltage output to the PMT was controlled with a variable voltage input of 0 to 5 V DC (GW laboratory DC power supply model:GPS-1850D). Data were acquired from the analog output of the PMT through a National Instruments NI SC-2345 signal conditioning connector block and NI 186623E-02 SCC-A102 isolated analog input connector (25-50 Hz sampling rate) and were stored on a Dell Dimension computer equipped with a National Instruments (NI) PCI 6221 card. Instrument control and data analysis were accomplished with programs developed in-house using NI-DAQmx and LabVIEW 7 Express software. All measurements were corrected for the spectral response of the Hamamatsu R928 PMT.

Reactions were initiated by the injection of 0.12 mL of 8.8 mM Mg-ATP into 8×50 polypropylene tubes containing 0.4 mL of 0.525-0.925 mM LH₂ in 25 mM glycylglycine buffer (pH 7.8) and 0.5-1 μg enzyme in CBA. The final concentrations of LH₂ and Mg-ATP were 0.4-0.7 and 2.0 mM, respectively, in a final volume of 0.525 mL. For integrated specific activities, light output was monitored for 15 min.

Steady-State Kinetic Constants.

Values of Kₘ and Vₘₐₓ for LH₂ and Mg-ATP were determined from bioluminescence activity assays in which measurements of maximal light intensities (bursts) were taken as estimates of initial velocities. Data for LH₂ and Mg-ATP were collected in 0.525 mL reactions in 25 mM glycylglycine buffer, pH 7.8, containing 0.5-1 μg of luciferase enzyme in CBA. The concentration of one substrate was maintained at saturation, while the other was varied (2 μM-1.5 mM for LH₂ and 10 μM-2 mM for Mg-ATP). Reactions were initiated by injection of solutions of the substrate being maintained at saturating concentration. Kinetic constants were determined using a nonlinear least squares method of the Enzyme Kinetics Pro software (SynTex), which fits data from the Michalis-Menten equation to a rectangular hyperbola. The corresponding k_{cat} values were obtained by dividing the Vₘₐₓ values by the amount (μmol) of each luciferase in the assay mixtures.

Rates of Half-Reactions.

The estimated rates of the oxidative half-reactions were based on bioluminescence activity assays using synthetic LH₂-AMP as the substrate. Assays (0.510 mL) in 50 mM glycylglycine buffer, pH 7.8, contained 0.1 mL aliquots of LH₂-AMP solution (final concentration 1.5-35 μM) in 10 mM sodium acetate, pH 4.5. Immediately, light reactions were initiated by injections of 10 μL of luciferase enzymes (0.4-1 μg in CBA). Kinetic constants were determined using a nonlinear least squares method of the Enzyme Kinetics Pro software (SynTex), which fits data from the Michalis-Menten equation to a rectangular hyperbola. The corresponding k_{cat} values were obtained by dividing the Vₘₐₓ values by the final amounts (μmol) of each luciferase in the assay mixtures.

The relative rates of adenylate formation were estimated (error ±15% of the value) by fluorescence-based assays of dehydroluciferyl-AMP (L-AMP) formation (Branchini 2000) using a Perkin Elmer LS55 luminescence spectrometer operated in the “time-drive” mode. Using an excitation wavelength of 350 nm, the luciferase-catalyzed formation of L-AMP from dehydroluciferin (L), initiated by the addition of Mg-ATP, was assessed by following the decrease in the intensity of the 440 nm fluorescence of the initial enzyme-L complex. The change in fluorescence was used to estimate the rates of L-AMP formation catalyzed by the luciferases. Assays (0.4 mL) in 50 mM Tris buffer, pH 7.4 contained 2.8 μM enzyme and 0.55 μM L. The initial fluorescence at 440 nm was recorded and then the decrease was monitored following the rapid injection of 50 μL solutions of varying concentrations of Mg-ATP in the same buffer. The rates of decrease (slopes) were calculated and used to determine the initial velocities for each Mg-ATP concentration. The data were fitted using a nonlinear least squares method of the Enzyme Kinetics Pro software (SynTex), which fits data from the Michalis-Menten equation to a rectangular hyperbola, and the Vₘₐₓ values were obtained. The corresponding k_{cat} values were obtained by dividing the Vₘₐₓ values (μmol/s) by the final amounts (μmol) of each luciferase in the assay mixtures.

Bioluminscence Activity Assays with Limiting LH₂.

Bioluminscence activity assays (0.4 mL) containing 65 nM LH₂ in 25 mM glycylglycine buffer (pH 7.8) and 3.25 μM of enzyme in CBA were initiated by the injection of 0.12 mL of 8.8 mM Mg-ATP in the same buffer. The final concentrations of enzyme, LH₂ and Mg-ATP were 2.5 μM, 50 nM and 2.0 mM, respectively, in a final volume of 0.52 mL. The light output was monitored (1 kHz sampling rate) for 30 s or until the initial signal intensity decreased by 99%. An additional aliquot of enzyme was added to the spent mixtures and emission intensity was monitored to ensure that the reactions were completed.

Heat Inactivation Studies.

Enzymes (1.2 mg/mL) in CBA were diluted to 0.1 mg/mL in 0.3 mL of 25 mM glycylglycine buffer (pH 7.8) at room temperature and then incubated at 37° C. Aliquots (2-3 μL) were removed over varying time periods and assayed for bioluminescence activity as described above.

Bioluminescence Emission Spectra.

Bioluminescence emission spectra were obtained using a Horiba Jobin-Yvon iHR imaging spectrometer equipped with a liquid N₂ cooled CCD detector and the excitation source turned off. Data were collected at 22° C. in a 0.8 mL quartz cuvette over the wavelength range 450-750 nm with the emission slit width set to 5 nm and were corrected for the spectral response of the CCD using a correction curve provided by the manufacturer. Reactions (0.52 mL in 25 mM glycylglycine buffer pH 7.8, 25 mM Tris pH 7.0, or 25 mM MES pH 6.5) containing 100 μM LH₂ and 2 mM Mg-ATP were initiated by the addition of 5 μL of enzyme in CBA (0.02-0.03 μM final concentration). The pH values were confirmed before and after spectra were obtained (Branchini 2010). The ratios of the emission intensities at 560 nm/610 nm at pH 6.0 and pH 6.5 were determined using values from the corrected data files.

### Example 1

### Chimeric PpyLit Protein

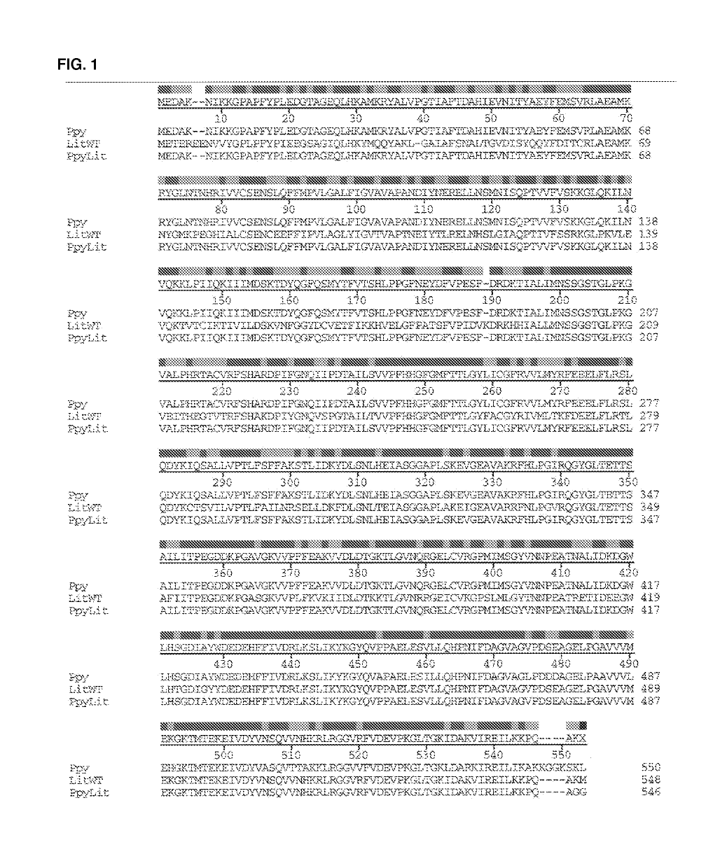

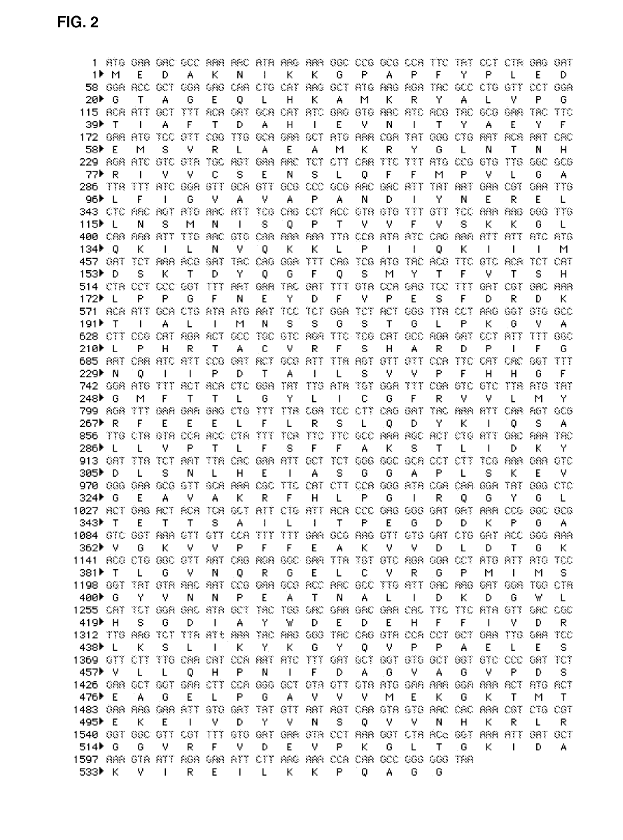

As part of an ongoing study on the identification of key residues in the catalysis of the bioluminescence half-reactions, we constructed PpyLit, a “control” chimeric firefly luciferase consisting of the N-domain (residues 1-436) of recombinant P. pyralis luciferase (PpyWT) joined to the C-domain of Luciola italica luciferase (LitWT) (24, 25) residues 442-548, LitWT numbering). The connecting hinge peptide 437ArgLeuLys439 is identical in both enzymes. Because of the high (76.6%) sequence identity between the C-domains, in effect, the Lit sequence introduced 27 changes, 23 amino acid substitutions and 4 deletions, into the full 550 amino acid PpyWT sequence. The amino acid sequences of PpyWT, LitWT and PpyLit are compared in FIG. 1 and the cDNA sequence of PpyLit is shown in FIG. 2.

The present invention is based on the surprising discovery that the chimeric PpyLit protein, which catalyzes yellow-green light emission (560 nm maximum), had unusually enhanced properties compared to recombinant PpyWT, native Luc isolated from firefly lanterns as well as LitWT. The activity data are presented in Table 2, shown below. Table 2 discloses ‘6×His’ as SEQ ID NO: 26.

TABLE 2
Bioluminiscent activity and thermostability of chimeric and wild type luciferases

| | Relative Specific | | | | | | | Thermal |
|---|---|---|---|---|---|---|---|---|
| | Activity^{a} | | | | | | Bioluminiscence | Inactivation^{e} |
| | Flash | | | K₁₀ (μM) | | k_{cat}/Kₘ | Emission | (min) at |
| Enzyme | Height | Integrated | k_{cat}(s⁻¹)^{b} | LH₂ | Mg-ATP | (mM⁻¹s⁻¹)^{c} | λₘₐₓ^{d} | 37° C. |
| PpyWt | 100 ± 4 | 100 ± 2 | 0.18 ± 0.01 | 15 ± 2 | 86 ± 7 | 2.09 ± 0.10 | 560 (70) | 18 ± 2 |
| Native Ppy | 99 ± 2 | 80 ± 2 | 0.17 ± 0.01 | 17 ± 1 | 90 ± 10 | 1.89 ± 0.13 | 563 (68) | n.d. |
| 6X-His-PpyWT | 98 ± 4 | 104 ± 8 | 0.18 ± 0.01 | 17 ± 1 | 93 ± 9 | 1.93 ± 0.11 | 561 (69) | 15 ± 1 |
| LitWT | 84 ± 2 | 163 ± 8 | 0.14 ± 0.01 | 90 ± 9 | 180 ± 14 | 0.78 ± 0.11 | 572 (93) | <2 ± 1 |
| LitPpy | 17 ± 1 | 73 ± 3 | 0.033 ± 0.001 | 54 ± 5 | 188 ± 10 | 1.18 ± 0.06 | 595 (90) | n.d. |
| PpyLit | 180 ± 10 | 200 ± 12 | 0.32 ± 0.02 | 25 ± 2 | 53 ± 5 | 6.04 ± 0.12 | 560 (69) | 4 ± 1 |
| Ppy5 | 170 ± 13 | 176 ± 17 | 0.31 ± 0.02 | 15 ± 2 | 72 ± 7 | 4.31 ± 0.12 | 559 (65) | 25 ± 6 |
| Ppy14 | 162 ± 14 | 131 ± 2 | 0.26 ± 0.02 | 11 ± 2 | 41 ± 3 | 6.34 ± 0.11 | 559 (64) | 5 ± 1 |
| PPy19 | 172 ± 6 | 208 ± 18 | 0.30 ± 0.01 | 15 ± 4 | 50 ± 5 | 5.99 ± 0.11 | 560 (63) | 3 ± 1 |
| PpyLit F465R | 190 ± 14 | 235 ± 3 | 0.34 ± 0.02 | 31 ± 3 | 99 ± 6 | 3.43 ± 0.10 | 558 (63) | 19 ± 1 |
| PpyLit I232A/E354K | 150 ± 3 | 181 ± 20 | 0.25 ± 0.01 | 24 ± 3 | 76 ± 4 | 3.29 ± 0.10 | 556 (65) | 150 ± 10 |
| PpyLit I351V/E354K | 170 ± 13 | 366 ± 30 | 0.30 ± 0.02 | 52 ± 5 | 80 ± 10 | 3.75 ± 0.15 | 555 (66) | 60 ± 5 |
| PpyLit I232A/ | 140 ± 5 | 385 ± 8 | 0.24 ± 0.01 | 51 ± 5 | 129 ± 14 | 1.86 ± 0.12 | 558 (65) | 80 ± 5 |
| I351V/E354K | | | | | | | | |
| PpyLit I351V/ | 160 ± 12 | 409 ± 18 | 0.29 ± 0.01 | 83 ± 3 | 94 ± 8 | 3.09 ± 0.09 | 558 (65) | 105 ± 9 |
| E354K/F465R | | | | | | | | |
| Ppy GR-TS | 45 ± 4 | 46 ± 4 | 0.081 ± 0.002 | 23 ± 3 | 163 ± 16 | 0.5 ± 0.1 | 546 (66) | 630 ± 50 |
| Ppy GR-TSLit | 43 ± 2 | 56 ± 6 | 0.079 ± 0.001 | 123 ± 9 | 466 ± 36 | 0.17 ± 0.07 | 550 (66) | 168 ± 15 |
| Ppy WT-T35/T351V | 150 ± 3 | 437 ± 2 | 0.25 ± 0.005 | 52 ± 6 | 79 ± 5 | 3.16 ± 0.07 | 559 (67) | >1440 ± 100 |
| Ppy 8284T | 24 ± 2 | 27 ± 2 | 0.05 ± 0.004 | 8 ± 1 | 79 ± 9 | 0.63 ± 0.14 | 614 (58) | 20 ± 2 |
| PpyLit 8284T | 64 ± 3 | 90 ± 7 | 0.10 ± 0.01 | 15 ± 2 | 89 ± 10 | 1.12 ± 0.15 | 614 (60) | 2 ± 1 |
| PpyLit 8284T/F465R | 62 ± 6 | 91 ± 9 | 0.10 ± 0.01 | 22 ± 3 | 60 ± 6 | 1.67 ± 0.14 | 614 (59) | 12 ± 2 |
| PpyLit S284T/ | 70 ± 6 | 185 ± 12 | 0.13 ± 0.01 | 49 ± 5 | 95 ± 9 | 1.36 ± 0.14 | 608 (60) | 16 ± 2 |
| I351V/E354K | | | | | | | | |
| PpyLit | 88 ± 11 | 171 ± 6 | 0.14 ± 0.01 | 84 ± 9 | 87 ± 11 | 1.61 ± 0.15 | 610 (58) | 87 ± 9 |
| I232A/8284T/ | | | | | | | | |
| I351V/E354I | | | | | | | | |
| PpyLit | 90 ± 6 | 231 ± 10 | 0.16 ± 0.01 | 82 ± 8 | 124 ± 15 | 1.29 ± 0.14 | 607 (61) | 157 ± 10 |
| 8284T/I351V/ | | | | | | | | |
| E354K/F465R | | | | | | | | |
| PpyLit | 85 ± 7 | 275 ± 16 | 0.13 ± 0.01 | 105 ± 8 | 117 ± 10 | 1.11 ± 0.12 | 607 (62) | 182 ± 10 |
| I232A/8284T/ | | | | | | | | |
| I351V/E354K/ | | | | | | | | |
| F465R | | | | | | | | |
| Ppy RE-TS | 31 ± 2 | 55 ± 4 | 0.056 ± 0.003 | 18 ± 2 | 68 ± 5 | 0.82 ± 0.09 | 610 (59) | 528 ± 50 |
| Ppy RE-T85/T351V | 67 ± 4 | 234 ± 2 | 0.12 ± 0.01 | 51 ± 8 | 93 ± 19 | 1.29 ± 0.14 | 611 (62) | 1440 ± 300 |
| Ppy RE-TSLit | 36 ± 1 | 77 ± 2 | 0.09 ± 0.003 | 51 ± 3 | 79 ± 9 | 1.13 ± 0.12 | 608 (62) | 252 ± 10 |
| Ppy RE13 | 59 ± 8 | 180 ± 3 | 0.11 ± 0.02 | 124 ± 14 | 289 ± 20 | 0.38 ± 0.10 | 619 (58) | 1380 ± 180 |
| HCO Ppy RE13 | 60 ± 2 | 154 ± 4 | — | — | — | — | 618 (58) | — |

^{a}Specific activities were obtained at pH 7.8 with LH₂ (400-700) μM) and Mg-ATP (2 mM) and are expressed relative to Ppy WT values, which are defined as 100. Integrated activities are based on total light emission in 15 min.
^{b}k_{cat} values were obtained by dividing the Vₘₐₓ values (in units of Einstein × 10⁻⁶ s⁻¹) obtained from the measurement of the Kₘ values for LH₂ by the amount in (micromoles) of each luciferase.
^{c}The k_{cat}/Kₘ was determined by dividing the k_{cat} by the Mg-ATP Kₘ value in (mM).
^{d}Bioluminescence emission maximum at pH 7.8 with the badwidth at full-width half-maximum show in parentheses. Emission maxima were obtained from two trials and the standard deviation is ±1 nm.
^{e}Time for the maximum initial activity to decay to 50% at 37° C. All activity value were obtained from at least three trials and are reported as means ± standard deviation.

Compared to PpyWT, the novel PpyLit chimera exhibited: (1) 1.8-fold enhanced flash-height (initial burst kinetics) specific activity; (2) 2.0-fold enhanced integration (total relative photon emission/15 min) specific activity; and 2.9-fold enhanced catalytic efficiency (kcat/Km ATP). Importantly, we have determined that the rates of both half reactions catalyzed by PpyLit (Scheme 1) proceed ˜1.35-fold faster than the corresponding PpyWT. Moreover, with excess PpyLit and a limiting amount of LH2 present, the there is a ˜1.4 greater photon yield than with PpyWT, showing that the bioluminescence quantum yield (Ando et al., 2008) has been considerably increased. These results indicate that the properties of PpyLit have been fundamentally altered. PpyLit is, however, less thermally stable than PpyWT (4 min vs 18 min half-lives at 37° C., respectively). However, PpyLit is sufficiently stable that assays performed at room temperature are not hindered by this difference.

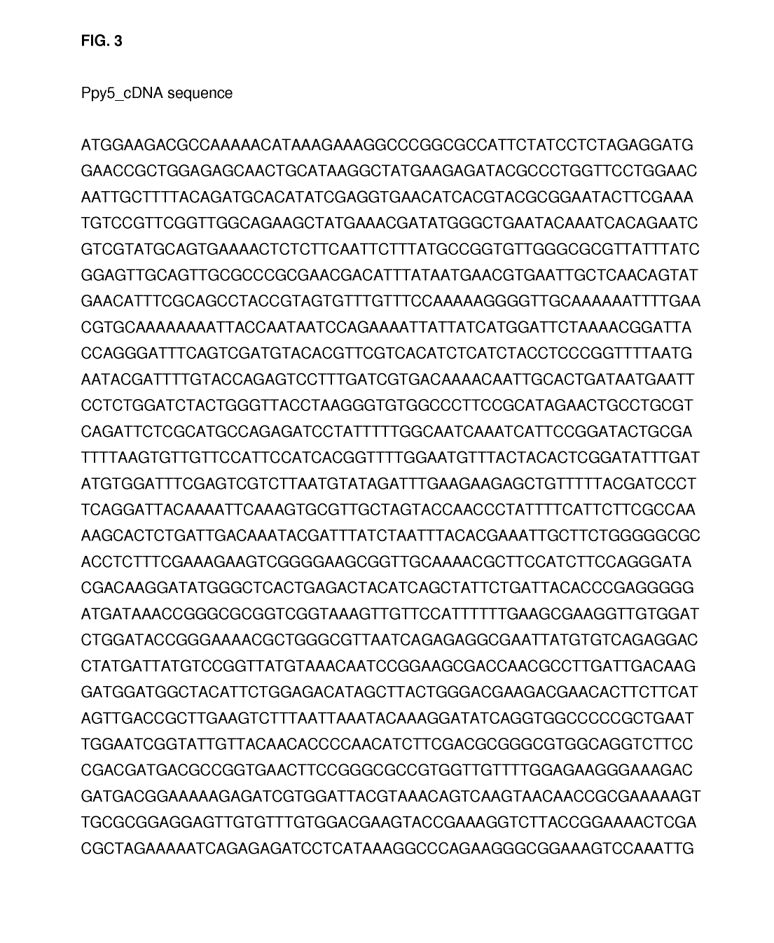

Additionally, we discovered that PpyLit was resistant to red shifting of light emission at low pH (6.0 to 7.2) compared to PpyWT and these data are presented in Table 3, shown below, and in FIG. 3. Table 3 discloses ‘6×His’ as SEQ ID NO: 26.

TABLE 3
Emission profiles and effect of pH on bioluminescence emission color.

| | Bioluminescence | pH Resistance to Color Shift ^{b} | | | Decay Time |
|---|---|---|---|---|---|
| Enzyme | Emission λₘₐₓ^{a} | pH 6.0 | pH 6.5 | Rise Time^{c} (s) | (min)^{d} |
| PpyWT | 560 (70) | 613 (0.25) | 608 (0.65) | 0.33 ± 0.04 | 0.21 ± 0.02 |
| Native Ppy | 563 (58) | 612 (0.25) | 605 (0.63) | 0.35 ± 0.03 | 0.20 ± 0.01 |
| 6X-His-PpyWT | 561 (69) | 614 (0.25) | 610 (0.68) | 0.36 ± 0.01 | 0.21 ± 0.02 |
| LitWT | 572 (93) | 615 (0.15) | 613 (0.48) | 0.21 ± 0.01 | 3.30 ± 0.07 |
| LitPpy | 595 (90) | 614 (0.16) | 610 (0.4) | 0.43 ± 0.04 | 8.3 ± 1 |
| PpyLit | 560 (69) | 609 (0.68) | 559 (1.46) | 0.29 ± 0.01 | 0.20 ± 0.01 |
| Ppy5 | 559 (65) | 613 (0.62) | 560 (1.52) | 0.36 ± 0.01 | 0.34 ± 0.01 |
| Ppy14 | 559 (64) | 615 (0.52) | 559 (1.33) | 0.35 ± 0.03 | 0.42 ± 0.04 |
| Ppy19 | 560 (68) | 611 (0.64) | 560 (1.51) | 0.29 ± 0.01 | 0.34 ± 0.02 |
| PpyLit F465R | 558 (63) | 565 (1.25) | 563 (2.07) | 0.32 ± 0.05 | 0.26 ± 0.08 |
| PpyLit I232A/E354K | 556 (65) | 560 (2.29) | 560 (2.45) | 0.32 ± 0.05 | 0.28 ± 0.01 |
| PpyLit I351V/E354K | 555 (66) | 559 (1.55) | 558 (1.87) | 0.34 ± 0.03 | 0.37 ± 0.03 |
| PpyLit I232A/I351V/E354K | 558 (65) | 560 (2.06) | 558 (2.36) | 0.37 ± 0.02 | 9.6 ± 0.3 |
| PpyLit I351V/E354K/F465R | 558 (65) | 559 (1.75) | 560 (2.36) | 0.36 ± 0.01 | 8.5 ± 0.4 |
| Ppy GR-TS | 551 (77) | 549 (3.45) | 552 (3.45) | 0.61 ± 0.03 | 0.22 ± 0.02 |
| Ppy GR-TSLit | 550 (66) | 550 (3.23) | 549 (3.48) | 0.8 ± 0.1 | 1.3 ± 0.5 |
| Ppy WT-TS5/I351V | 559 (67) | 563 (1.79) | 561 (2.23) | 0.42 ± 0.03 | 7.1 ± 0.30 |
| Ppy S284T | 614 (58) | 618 (0.064) | 616 (0.065) | 0.25 ± 0.02 | 0.34 ± 0.02 |
| PpyLit S284T | 614 (60) | 614 (0.067) | 615 (0.065) | 0.34 ± 0.03 | 2.5 ± 0.07 |
| PpyLit S284T/F465R | 614 (59) | 614 (0.07) | 614 (0.06) | 0.3 ± 0.03 | 2.31 ± 0.01 |
| PpyLit S284T/I351V/E354K | 608 (60) | 612 (0.07) | 614 (0.08) | 0.32 ± 0.01 | 6.5 ± 0.2 |
| PpyLit I232A/S284T/I351V/E354I | 610 (58) | 612 (0.06) | 614 (0.05) | 0.32 ± 0.01 | 4.4 ± 0.3 |
| PpyLit | 607 (61) | 614 (0.07) | 613 (0.07) | 0.36 ± 0.01 | 8.2 ± 0.3 |
| S284T/I351V/E354K/F465R | | | | | |
| PpyLit | 607 (62) | 615 (0.09) | 612 (0.10) | 0.40 ± 0.01 | 8.3 ± 0.1 |
| I232A/S284T/I351V/E354K/F465R | | | | | |
| Ppy RE-TS | 610 (59) | 614 (0.07) | 612 (0.07) | 0.45 ± 0.03 | 0.23 ± 0.02 |
| Ppy RE-TS5/I351V | 611 (62) | 615 (0.11) | 615 (0.09) | 0.40 ± 0.03 | 8.0 ± 0.7 |
| Ppy RE-TSLit | 608 (62) | 613 (0.08) | 615 (0.09) | 0.40 ± 0.01 | 6.6 ± 0.2 |
| Ppy RE13 | 619 (58) | 624 (0.05) | 618 (0.10) | 0.48 ± 0.01 | 7.5 ± 1 |
| HCO Ppy RE13 | 618 (58) | — | — | 0.46 ± 0.03 | 7.3 ± 5 |

^{a}Bioluminescence emission maximum at pH 7.8 with the bandwidth at full-width half-maximum shown in parentheses.
^{b} The bioluminescence emission maximum at the indicated pH with the ratio of the maxima (560:610 nm) shown in parentheses. Bioluminescence emission maxima were obtained from two trials and the standard deviation is ±1 nm.
^{c}Bioluminescence rise time to maximum intensity.
^{d}Time for the bioluminescence signal to decay to 10% of the maximum value. Rise and Decay times were obtained from at least three trials and are reported as means ± standard deviation.

The bathochromic shift below ˜pH 7.2 is a characteristic the true firefly luciferases; while the railroad worm and click beetle luciferases do not red shift in the pH 6-8.5 pH region (White et al., 1971; Ando et al. 2008; Viviani et al., 2002). The luminescence intensity of all beetle luciferases does decrease as the acidity of the medium increases. In addition to demonstrating the effect of pH on bioluminescence color in FIG. 3, we express the bathochromic shift as the ratio of the emission intensities 560 nm/610 nm at pH 6.0 and 6.5 in Table 3.

Because of the importance and widespread use of native and recombinant P. pyralis luciferase and mutants derived from it, the presently described PpyLit chimeric proteins should be a replacement, offering superior sensitivity and stability in applications where Luc and some variants of it are currently used, particularly in the detection of ATP. Moreover, by introducing our previously discovered (Branchini et al, 2005) Ser284Thr mutation into PpyLit, PpyLit F465R, PpyLit I351V/E354K, I232A/I351V/E354I, I351V/E354K/F465R and I232A/I351V/E354K/F465R, we have conferred upon PpyLit the ability to emit red light (607-614 nm emission maximum, Table 3 & FIG. 3). To the best of our knowledge, the specific activity of the Ser284Thr containing PpyLit enzymes makes it the brightest red emitting luciferases known as the emission intensity exceeds that of Ppy S284T by 2.7-fold while producing 3.3-fold more total light in 15 min (Table 2). While PpyLit S284T lacks good thermostability, promising improvements in this property already have been realized without sacrificing emission intensity (for example, PpyLit S284T/I351V/E354K, Table 2). Moreover, substantial increases (to ˜3 h at 37° C.) in thermostability with concomitant ˜33-40% further enhanced specific activity were achieved with PpyLit S284T/I351V/E354K/F465R and PpyLit I232A/S284T/I351V/E354K/F465R (Table 2). We note that with the red emitting luciferases, pH sensitivity is not relevant because they rarely color shift with pH. Studies are in progress to improve thermostability while maintaining the advantageous enhanced emission sensitivity.

### Improvements of PpyLit Properties.

Because of the importance and widespread use of PpyWT and mutants derived from it, our basis of enzyme physical property comparison in the Tables and Figures is PpyWT. Note that there are only very minor differences between the properties of the recombinant and native enzymes (Table 2).

Prior to undertaking the mutagenesis studies to improve the properties of PpyLit, we made the corresponding LitPpy chimeric protein and its specific activity by both measurements is much lower than both LitWT and PpyWT, only 17% flash height and 73% integration-based compared to the P. pyralis enzyme. In fact, we made approximately 30 or more variants of PpyWT containing 1 to 19 mutations at the 27 varying positions of the LitWT C-domain. Only one of these mutants, Ppy19, which contains 19 amino acid differences in the contiguous region 450-543, essentially maintains all of the properties of PpyLit. While all of these 19 amino acid changes were necessary to produce the specific activity effects observed with PpyLit, the pH resistance property was fully realized with only the 14 changes contained in Ppy14 (Tables 3). We note that all of the 14 amino acid changes may not be required to produce the resistance to color shifting observed with PpyLit. We note also that as a result of introducing the changes discussed below that were designed to produce enhanced thermostability, we also further improved the resistance to low pH color shifting beyond what was observed for PpyLit. In fact, the enzymes PpyLitI232A/I351V/E354K and PpyLit I351V/E354K/F465R are essentially completely resistant to this pH effect (Table 3 and FIG. 3).

The strategy to improve the stability properties of PpyLit and PpyLit S284T was based on introducing the fewest number of mutations chosen from the following: Thr214Ala, Ala215Leu, Ile232Ala, Phe295Leu, Glu354Lys and Phe465Arg. Based on random mutagenesis studies, Tisi and coworkers had reported (Tisi et al., 2002; Baggett et al., 2004) a highly thermostable Luc variant containing the five mutations spanning residues 214-354, and this group also described (Law et al., 2006) the effects of the position 465 change We had successfully extended (Branchini et al., 2007a; Mezzanote et al., 2010) this work by making previously developed (Branchini et al., 2005) blue- and red-shifted luciferases more thermostable and pH change resistant using Tisi's five N-terminal domain amino acid changes. Additionally, we investigated the effects of the E354I mutation (White, et al., 1996) that we adapted in some of our previous work on thermostable luciferase development (Branchini, 2010, Anal Biochem 396, 290-297). In the course of our work, we had evaluated the effects of the individual mutations to determine to what extent they improved thermal characteristics and decreased specific activity, a drawback accompanying some of these amino acid changes. We therefore only investigated three of the six mutations (Ile232Ala, Glu354Lys and Phe465Arg) plus a fourth (Ile351Val) that we had previously determined (Branchini et al., 2003) enhanced the specific activity of PpyWT, intending that the latter change could offset some of the expected loss of activity that accompanied improved thermostability as we had recently observed in a separate study (Branchini et al., 2010).

We have made the thermostability of PpyLit comparable to that of PpyWT (18 min half life at 37° C.) by introducing a single point mutation Phe465Arg to create PpyLit F465R (19 min half life at 37° C.). The mutation slightly improved specific activity and dramatically improved pH resistance to color shifting to the point where there is only slight shifting at pH 6.5 (Tables 2 & 3, FIG. 3). If it is desirable to have a stable luciferase with enhanced activity with the fewest additional changes in PpyLit, PpyLit F465R is a preferred luciferase. If one desires the greatest combined thermo- and pH stability at the modest expense of some of the intensity enhancement, PpyLit I232A/E354K is a preferred luciferase. In certain preferred embodiments, the best balance of stability and activity is provided by PpyLit I351V/E354K (Tables 2 & 3, FIG. 3).

Not all of the attempts to combine mutations resulted in the anticipated improvements in activity and/or thermostability. For example, adding the amino acid change F465R to PpyLit I232A/E354 failed to improve the enzyme's thermostability and actually unexpectedly reduced this property. Among the red-emitting luciferases PpyLit S284T/F465R provides the longest wavelength emission (614 nm) and sufficient thermostability to be used in room temperature applications. PpyLit S284T/I351V/E354K/F465R has outstanding thermostability at 37° C. and emission enhanced ˜3.5-fold (compared to Ppy S284T) and PpyLit I232A/S284T/I351V/E354I has the best combination of activity, emission maximum and thermostability (Tables 2 & 3). The emission intensity of all the Ser284Thr containing luciferases is exceptional as is clear from the improvement over Ppy S284T already one of the brightest red enzymes reported.

### INCORPORATION BY REFERENCE

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

### REFERENCES

Branchini, B. R., Magyar, R. A., Murtiashaw, M. H., and Portier, N. C. (2001) The role of active site residue arginine 218 in firefly luciferase bioluminescence, Biochemistry 40, 2410-2418.
Branchini, B. R., Southworth, T. L., DeAngelis, J. P., Roda, A., and Michelini, E. (2006) Luciferase from the Italian firefly Luciola italica: Molecular cloning and expression, Comparative Biochemistry and Physiology B-Biochemistry & Molecular Biology 145, 159-167.
Branchini, B. R., Magyar, R. A., Murtiashaw, M. H., Anderson, S. M., and Zimmer, M. (1998) Site-directed mutagenesis of histidine 245 in firefly luciferase: A proposed model of the active site, Biochemistry 37, 15311-15319.
Branchini, B. R., Ablamsky, D. M., Murtiashaw, M. H., Uzasci, L., Fraga, H., and Southworth, T. L. (2007a) Thermostable red and green light-producing firefly luciferase mutants for bioluminescent reporter applications, Anal. Biochem. 361, 253-262.
Branchini, B. R., Rosenberg, J. C., Fontaine, D. M., Southworth, T. L., Behney, C. E., and Uzasci, L. (2011) Bioluminescence Is Produced from a Trapped Firefly Luciferase Conformation Predicted by the Domain Alternation Mechanism, JACS 133, 11088-11091.
Branchini, B. R., Ablamsky, D. M., Rosenman, J. M., Uzasci, L., Southworth, T. L., and Zimmer, M. (2007b) Synergistic mutations produce blue-shifted bioluminescence in firefly luciferase, Biochemistry 46, 13847-13855.
Branchini, B. R., Murtiashaw, M. H., Magyar, R. A., and Anderson, S. M. (2000) The role of lysine 529, a conserved residue of the acyl-adenylate-forming enzyme superfamily, in firefly luciferase, Biochemistry 39, 5433-5440.
Branchini, B. R., Ablamsky, D. M., and Rosenberg, J. C. (2010) Chemically Modified Firefly Luciferase Is an Efficient Source of Near-Infrared Light, Bioconjugate Chem. 21, 2023-2030.
Gulick, A. M. (2009) Conformational Dynamics in the Acyl-CoA Synthetases, Adenylation Domains of Non-ribosomal Peptide Synthetases, and Firefly Luciferase, Acs Chemical Biology 4, 811-827.
Branchini, B. R., Rosenberg, J. C., Fontaine, D. M., Southworth, T. L., Behney, C. E., and Uzasci, L. (2011) Bioluminescence Is Produced from a Trapped Firefly Luciferase Conformation Predicted by the Domain Alternation Mechanism, JACS 133, 11088-11091.
Strieker, M., Tanovic, A., and Marahiel, M. A. (2010) Nonribosomal peptide synthetases: structures and dynamics, Current Opinion in Structural Biology 20, 234-240.
Conti, E., Franks, N. P., and Brick, P. (1996) Crystal structure of firefly luciferase throws light on a superfamily of adenylate-forming enzymes, Structure 4, 287-298.
Gulick, A. M., Starai, V. J., Horswill, A. R., Homick, K. M., and Escalante-Semerena, J. C. (2003) The 1.75 Å crystal structure of acetyl-CoA synthetase bound to adenosine-5′-propylphosphate and coenzyme A, Biochemistry 42, 2866-2873.
White, E. H., Rapaport, E., Seliger, H. H., and Hopkins, T. A. (1971) Chemi- and bioluminescence of firefly luciferin. Efficient chemical production of electronically excited states, Bioorg. Chem. 1, 92-122.
DeLuca, M. (1976) Firefly luciferase, Adv. Enzymol. 44, 37-68.
Fraga, H., Fernandes, D., Fontes, R., and Esteves da Silva, J. C. G. (2005) Coenzyme A affects firefly luciferase luminescence because it acts as a substrate and not as an allosteric effector, FEBS Journal 272, 5206-5216.
Ozawa, T. (2006) Designing split reporter proteins for analytical tools, Anal. Chim Acta 556, 58-68.
Binkowski, B. F., Butler, B. L., Stecha, P. F., Eggers, C. T., Otto, P., Zimmerman, K., Vidugiris, G., Wood, M. G., Encell, L. P., Fan, F., and Wood, K. V. (2011) A Luminescent Biosensor with Increased Dynamic Range for Intracellular cAMP, Acs Chemical Biology 6, 1193-1197.
Roda, A., and Guardigli, M. (2012) Analytical chemiluminescence and bioluminescence: latest achievements and new horizons, Analytical and Bioanalytical Chemistry 402, 69-76.
Ohmiya, Y., Hirano, T., and Ohashi, M. (1996) The structural origin of the color differences in the bioluminescence of firefly luciferase, FEBS Lett. 384, 83-86.
Viviani, V. R., and Ohmiya, Y. (2000) Bioluminescence color determinants of Phrixothrix railroad-worm luciferases: Chimeric luciferases, site-directed mutagenesis of Arg 215 and guanidine effect, Photochem. Photobiol. 72, 267-271.
Viviani, V. R., da Silva Neto, A. J., and Ohmiya, Y. (2004) The influence of the region between residues 220 and 344 and beyond in Phrixotrix railroad worm luciferases green and red bioluminescence, Protein Engineering Design & Selection 17, 113-117.
Hirokawa, K., Kajiyama, N., and Murakami, S. (1999) Enhanced performance of firefly luciferase by gene chimerization and random mutagenesis, In Bioluminescence and Chemiluminescence: Perspectives for the 21st Century (Roda, A., Pazzagli, M., Kricka, L. J., and Stanley, P. E., Eds.), pp 412-415, John Wiley & Sons, Chichester.
Hirokawa, K., Kajiyama, N., and Murakami, S. (2002) Improved practical usefulness of firefly luciferase by gene chimerization and random mutagenesis, Biochim Biophys. Acta 1597, 271-279.
Oba, Y., Tanaka, K., and Inouye, S. (2006) Catalytic properties of domain-exchanged chimeric proteins between firefly luciferase and Drosophila fatty acyl-CoA synthetase CG6178, Biosci., Biotechnol., Biochem. 70, 2739-2744.
Zako, T., Ayabe, K., Aburatani, T., Kamiya, N., Kitayama, A., Ueda, H., and Nagamune, T. (2003) Luminescent and substrate binding activities of firefly luciferase N-terminal domain, Biochimica Et Biophysica Acta-Proteins and Proteomics 1649, 183-189.
Ayabe, K., Zako, T., and Ueda, H. (2005) The role of firefly luciferase C-terminal domain in efficient coupling of adenylation and oxidative steps, FEBS Lett. 579, 4389-4394.
Fujii, H., Noda, K., Asami, Y., Kuroda, A., Sakata, M., and Tokida, A. (2007) Increase in bioluminescence intensity of firefly luciferase using genetic modification, Anal. Biochem. 366, 131-136.
Nakajima, Y., Yamazaki, T., Nishii, S., Noguchi, T., Hoshino, H., Niwa, K., Viviani, V. R., and Ohmiya, Y. (2010) Enhanced Beetle Luciferase for High-Resolution Bioluminescence Imaging, Plos One 5.
Noda, K., Goto, H., Murakami, Y., Ahmed, A. B. F., and Kuroda, A. (2010) Endotoxin assay by bioluminescence using mutant firefly luciferase, Anal. Biochem. 397, 152-155.
Noda, K., Matsuno, T., Fujii, H., Kogure, T., Urata, M., Asami, Y., and Kuroda, A. (2008) Single bacterial cell detection using a mutant luciferase, Biotechnol. Lett 30, 1051-1054.
Branchini, B. R., Southworth, T. L., DeAngelis, J. P., Roda, A., and Michelini, E. (2006) Luciferase from the Italian firefly Luciola italica: Molecular cloning and expression, Comparative Biochemistry and Physiology B-Biochemistry & Molecular Biology 145, 159-167.
Branchini, B. R., Southworth, T. L., Deangelis, J. P., Roda, A., and Michelini, E. (2007) Cloning, sequence, bioluminescence and mutagenesis of luciferase of Luciola italica, p 23 pp., Connecticut College, USA.
Ando, Y., Niwa, K., Yamada, N., Enomot, T., Irie, T., Kubota, H., Ohmiya, Y., and Akiyama, H. (2008) Firefly bioluminescence quantum yield and colour change by pH-sensitive green emission, Nat Photonics 2, 44-47.
Viviani, V. R. (2002) The origin, diversity, and structure function relationships of insect luciferases, Cell. Mol. Life Sci. 59, 1833-1850.
Branchini, B. R., Southworth, T. L., Khattak, N. F., Michelini, E., and Roda, A. (2005) Red- and green-emitting firefly luciferase mutants for bioluminescent reporter applications, Anal. Biochem. 345, 140-148.
Tisi, L. C., White, P. J., Squinell, D. J., Murphy, M. J., Lowe, C. R., and Murray, J. A. H. (2002) Development of a thermostable firefly luciferase, Anal. Chim Acta 457, 115-123.
Baggett, B., Roy, R., Momen, S., Morgan, S., Tisi, L., Morse, D., and Gillies, R. J. (2004) Thermostability of firefly luciferases affects efficiency of detection by in vivo bioluminescence, Molecular Imaging 3, 324-332.
Law, G. H. E., Gandelman, O. A., Tisi, L. C., Lowe, C. R., and Murray, J. A. H. (2006) Mutagenesis of solvent-exposed amino acids in Photinus pyralis luciferase improves thermostability and pH-tolerance, Biochem. J 397, 305-312.
Branchini, B. R., Ablamsky, D. M., Murtiashaw, M. H., Uzasci, L., Fraga, H., and Southworth, T. L. (2007) Thermostable red and green light-producing firefly luciferase mutants for bioluminescent reporter applications, Anal. Biochem. 361, 253-262.
Mezzanotte, L., Fazzina, R., Michelini, E., Tonelli, R., Pession, A., Branchini, B., and Roda, A. (2010) In Vivo Bioluminescence Imaging of Murine Xenograft Cancer Models with a Red-shifted Thermostable Luciferase, Mol Imaging Biol 12, 406-414.
Branchini, B. R., Southworth, T. L., Murtiashaw, M. H., Boije, H., and Fleet, S. E. (2003) A mutagenesis study of the putative luciferin binding site residues of firefly luciferase, Biochemistry 42, 10429-10436.
Branchini, B. R., Ablamsky, D. M., Davis, A. L., Southworth, T. L., Butler, B., Fan, F., Jathoul, A. P., and Pule, M. A. (2010) Red-emitting luciferases for bioluminescence reporter and imaging applications, Anal. Biochem. 396, 290-297.
White, P. J. Squinell, D. J., Arnaud, P., Lowe C. R., and Murray, J. A. (1996) Improved thermostability of the North American firefly luciferase: saturation mutagenesis at position 354, Biochem J (Pt2), 343-350.

1. A firefly luciferase comprising an N-terminal amino acid domain from a first firefly luciferase and a C-terminal amino acid domain from a second firefly luciferase.

2. A firefly luciferase comprising an N-terminal amino acid domain from a first firefly luciferase and a C-terminal amino acid domain from a second firefly luciferase, wherein the N-terminal amino acid domain is from Photinius pyralis (P. pyralis; Ppy) luciferase and the C-terminal amino acid domain is from Luciola italica (L. italica; Lit) luciferase.

3. The firefly luciferase of claim 2, further comprising a linker peptide.

4. The firefly luciferase of claim 3, wherein the linker peptide is a tripeptide linker.

5. The firefly luciferase of claim 3, wherein the linker peptide comprises ArgLeuLys or ArgTyrLys.

6. The firefly luciferase of claim 3, wherein the linker peptide further comprises a mutation.

7. The firefly luciferase of claim 3, wherein the linker peptide comprises residues 437-439 of SEQ ID NO:4.

8. The firefly luciferase of claim 2, wherein the L italica luciferase comprises the amino acid sequence of SEQ ID NO:2 or SEQ ID NO:6.

9. The firefly luciferase of claim 8, wherein SEQ ID NO:2 is encoded by the nucleic acid sequence of SEQ ID NO: 1.

10. The firefly luciferase of claim 8, wherein SEQ ID NO:6 is encoded by the nucleic acid sequence of SEQ ID NO: 5.

11. The firefly luciferase of claim 1, wherein the C-terminal amino acid domain comprises the amino acid sequence of SEQ ID NO:2 or SEQ ID NO:6, and the N-terminal amino acid domain is from a second firefly luciferase.

12. The firefly luciferase of claim 11, wherein the N-terminal amino acid domain is from P. pyralis luciferase.

13. The firefly luciferase of claim 2, wherein the P. pyralis luciferase comprises the amino acid sequence of SEQ ID NO:4.

14. The firefly luciferase of claim 13, wherein SEQ ID NO:4 is encoded by the nucleic acid sequence of SEQ ID NO:3.

15. The firefly luciferase of claim 1, wherein the N-terminal amino acid domain comprises the amino acid sequence of SEQ ID NO:4 and the C-terminal amino acid domain is from a second firefly luciferase.

16. The firefly luciferase of claim 15, wherein the C-terminal amino acid domain is from L. italica luciferase.

17. The firefly luciferase of claim 2, wherein the P. pyralis luciferase comprises an N-terminal extension peptide.

18. The firefly luciferase of claim 17, wherein the N-terminal extension peptide is selected from the group consisting of: GPLGS and HisTag.

19. The firefly luciferase of claim 12, wherein the P. pyralis luciferase comprises an N-terminal extension peptide.

20. The firefly luciferase of claim 19, wherein the N-terminal extension peptide is selected from the group consisting of: GPLGS and HisTag.

21. The firefly luciferase of claim 2, wherein the N-terminal domain comprises residues 1-436 of SEQ ID NO: 4, and the C-terminal domain comprises residues 442-548 of SEQ ID NO:6.

22. The firefly luciferase of claim 2 comprising the nucleic acid sequence of SEQ ID NO:7.

23. The firefly luciferase of claim 2 comprising the amino acid sequence of SEQ ID NO:8.

24. A firefly luciferase comprising the amino acid sequence of SEQ ID NO:4.

25. The firefly luciferase of claim 24, further comprising one or more amino acid changes selected from the group consisting of: A450P, I457V, L472V, D475S, D476E, A482G, L487M, H489K, A503N, T507V, T508N, A509H, K511R and V517R.

26. The firefly luciferase of claim 24, further comprising one or more amino acid changes selected from the group consisting of: A450P, I457V, L472V, D475S, D476E, A482G, L487M, H489K, A503N, T507V, T508N, A509H, K511R, V517R, L530I, K534V, 1540K, A542P and K543Q.

27. The firefly luciferase of claim 24, further comprising one or more amino acid changes selected from the group consisting of: I457V, A482G, H489K, A503N and K543Q.

28. The firefly luciferase of claim 27, comprising the amino acid sequence of SEQ ID NO 10.

29. The firefly luciferase of claim 28, wherein SEQ ID NO:10 is encoded by the nucleic acid sequence of SEQ ID NO:9.

30. The firefly luciferase of claim 23, further comprising the amino acid change F465R.

31. The firefly luciferase of claim 23, further comprising an amino acid change I232A/E354K.

32. The firefly luciferase of claim 23, further comprising an amino acid change I351V/E354K.

33. The firefly luciferase of claim 23, further comprising an amino acid change I232A/I351V/E354K.

34. The firefly luciferase of claim 23, further comprising an amino acid change I351V/E354K/F465R.

35. The firefly luciferase of claim 23, further comprising the amino acid change S284T.

36. The firefly luciferase of claim 23, further comprising the amino acid change S284T/F465R.

37. The firefly luciferase of claim 23, further comprising the amino acid change S284T/I351V/E354K.

38. The firefly luciferase of claim 23, further comprising the amino acid change I232A/S284T/I351V/E354I.

39. The firefly luciferase of claim 23, further comprising the amino acid change S284T/I351V/E354K/F465R.

40. The firefly luciferase of claim 23, further comprising the amino acid change I232A/S284T/I351V/E354K/F465R.

41. The firefly luciferase of claim 23, further comprising one or more amino acid changes selected from the group consisting of: F465R, I232A, E354K, I351V, I232A, S284T, E354I, T214A, A215L, and F295L.

42. The firefly luciferase of claim 15, further comprising the amino acid change T214A/A215L/I232A/V241I/G246A/F250S/F295L/E354K.

43. The firefly luciferase of claim 15, further comprising the amino acid change T214A/A215L/I232A/S284T/F295L/E354K.

44. The firefly luciferase of claim 15, further comprising one or more amino acid changes selected from the group consisting of: T214A, A215L, I232A, V241I, G246A, F250S, F295L, E354K, S284T and I351V.

45. A firefly luciferase comprising the amino acid sequence of SEQ ID NO: 22, with one or more amino acid changes selected from the group consisting of: I457V, A482G, H489K, A503N, K543Q and I351V.

46. The firefly luciferase of claim 45, comprising the amino acid sequence of SEQ ID NO:22 with the amino acid change I457V/A482G/H489K/A503N/K543Q/I351V.

47. The firefly luciferase of claim 46, comprising the amino acid sequence of SEQ ID NO 12.

48. The firefly luciferase of claim 47, wherein SEQ ID NO:12 is encoded by the nucleic acid sequence of SEQ ID NO:11.

49. A firefly luciferase comprising the amino acid sequence of SEQ ID NO: 24, with one or more amino acid changes selected from the group consisting of: I457V, A482G, H489K, A503N, K543Q and I351V.

50. The firefly luciferase of claim 49, comprising the amino acid sequence of SEQ ID NO:24 with the amino acid change I457V/A482G/H489K/A503N/K543Q/I351V.

51. The firefly luciferase of claim 50, comprising the amino acid sequence of SEQ ID NO 14.

52. The firefly luciferase of claim 51 wherein SEQ ID NO:14 is encoded by the nucleic acid sequence of SEQ ID NO:13.

53. A firefly luciferase comprising the amino acid sequence of SEQ ID NO: 20, with one or more amino acid changes selected from the group consisting of: I457V, R465F, A482G, H489K, A503N and K543Q.

54. The firefly luciferase of claim 53, comprising the amino acid sequence of SEQ ID NO: 20 with the amino acid change I457V/R465F/A482G/H489K/A503N/K543Q.

55. The firefly luciferase of claim 54, comprising the amino acid sequence of SEQ ID NO:16.

56. The firefly luciferase of claim 55, wherein SEQ ID NO:16 is encoded by the nucleic acid sequence of SEQ ID NO:15.

57. The firefly luciferase of claim 55, wherein the sequence is codon optimized.

58. A codon optimized firefly lucerifase comprising the amino acid sequence of SEQ ID NO:18.

59. The codon optimized firefly luciferase of claim 58, wherein SEQ ID NO:18 is encoded by the nucleic acid sequence of SEQ ID NO:17.

60. The firefly luciferase of claim 1, wherein the thermostability is increased compared to the P. pyralis luciferase.

61. The firefly luciferase of claim 1, wherein the resistance to color shifting is increased compared to the P. pyralis luciferase.

62. The firefly luciferase of claim 1, wherein the flash-height activity, integration specific activity or catalytic efficiency is increased.

63. The firefly luciferase of claim 1, wherein the chimeric luciferase is resistant to red shifting of light emission at low pH.

64. The firefly luciferase of claim 1, wherein the chimeric firefly luciferase has the ability to emit red light at a wavelength of about 607 to 614 nm.

65. The firefly luciferase of claim 1, further comprising an N-terminal peptide extension.

66. An expression vector comprising a nucleic acid sequence encoding the firefly luciferase of claim 1.

67. The expression vector of claim 66, wherein the firefly luciferase is expressed from a mammalian codon optimized gene.

68. The expression vector of claim 66, further comprising a promoter sequence.

69. A cell comprising the expression vector of claim 68.

70. A kit comprising the chimeric luciferase of claim 1.

71. A method for detection of transcriptional activity in a cell comprising introducing the expression vector of claim 66 into a cell, wherein the expression vector comprises a promoter of interest, and detecting the light emission, wherein the detection of light emission indicates transcriptional activity.

72. A method for in vivo imaging comprising introducing the firefly luciferase of claim 1 into a cell of a living animal and detecting the light emission.

73. A method for detecting the amount of ATP in a sample comprising:
contacting a sample with a firefly luciferases of claim 1; and
detecting ATP.


SEQUENCE LISTING (72 sequences)

SEQ ID NO: 1 - DNA, 1643 bp, Luciola italica
atggaaacggaaagggaggaaaatgttgtatatggccctctgccattctaccccattgaagaaggatcagctggaattcagttgcataagtacatgcaacaatatgccaaacttggagcaattgcttttagtaacgcccttactggagtggatatttcttaccaacaatactttgatattacatgtcgtttagctgaggcaatgaaaaactacggtatgaaaccggaaggacatattgctttgtgcagtgaaaattgtgaagaatttttcatccctgtgcttgctggtctttacattggagtaactgtcgcacctactaatgaaatttacacattgcgtgaacttaatcacagtttgggcatcgcacaaccaactattgtattcagctccagaaaaggcttacctaaagttttagaagtgcaaaaaacagttacatgcatcaaaacaattgttattttagatagtaaagtaaactttggaggctacgattgtgtggaaacttttattaagaaacatgtagaattaggttttccagcaactagctttgtacccattgatgtaaaggaccgtaaacatcacattgctttgcttatgaattcttctggctctactggtttacctaaaggtgtagagattacccacgaaggaacagttacaagattctcacacgctaaggatccaatttacggaaaccaagtttcacctggtactgctattttaactgtcgttccgttccatcatggatttggaatgtttaccactttaggatactttgcttgtggataccgtattgtaatgttaacaaaattcgatgaagaactatttttgagaactttgcaagattataagtgtaccagtgttattcttgtaccaacgttatttgctattctcaacaggagtgaattgctcgataagttcgatttatctaatctaactgaaattgcttctggtggagctcctttggcaaaagaaattggtgaagcagtcgctagaagatttaatctacccggtgtccgtcagggttacggattgacagaaacgacatctgcatttattattaccccagaaggtgatgataaacctggagcatctggaaaagtagtacccttattcaaagtaaaaattattgatcttgacactaaaaaaactttgggtgtcaaccgacgaggagagatctgtgtaaaaggtccgagtcttatgttaggctacacaaacaatccggaagcaacaagagaaactattgatgaagagggttggtgcacaccggagatattggatattacgacgaagacgaacatttcttcattgtagatcgtttgaaatcattaatcaaatacaaggggtaccaggtaccacctgctgaattggaatccgttcttttgcaacatccaaatatctttgatgctggtgtggctggtgtccccgattctgaagctggtgaacttccaggggctgtagttgtaatggaaaaaggaaaaactatgactgaaaaggaaattgtggattatgttaatagtcaagtagtgaaccacaaacgtctgcgtggtggcgttcgttttgtggatgaagtacctaaaggtctaactggtaaaattgatgctaaagtaattagagaaattcttaagaaaccacaagccaagatg

SEQ ID NO: 2 - protein, 548 aa, Luciola italica
METEREENVVYGPLPFYPIEEGSAGIQLHKYMQQYAKLGAIAFSNALTGVDISYQQYFDITCRLAEAMKNYGMKPEGHIALCSENCEEFFIPVLAGLYIGVTVAPTNEIYTLRELNHSLGIAQPTIVFSSRKGLPKVLEVQKTVTCIKTIVILDSKVNFGGYDCVETFIKKHVELGFPATSFVPIDVKDRKHHIALLMNSSGSTGLPKGVEITHEGTVTRFSHAKDPIYGNQVSPGTAILTVVPFHHGFGMFTTLGYFACGYRIVMLTKFDEELFLRTLQDYKCTSVILVPTLFAILNRSELLDKFDLSNLTEIASGGAPLAKEIGEAVARRFNLPGVRQGYGLTETTSAFIITPEGDDKPGASGKVVPLFKVKIIDLDTKKTLGVNRRGEICVKGPSLMLGYTNNPEATRETIDEEGWLHTGDIGYYDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAKM

SEQ ID NO: 3 - DNA, 1650 bp, Photinius pyralis
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 4 - protein, 550 aa, Photinius pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 5 - DNA, 1644 bp, Luciola italica
atggaaacggaaagggaggaaaatgttgtatatggccctctgccattctaccccattgaagaaggatcagctggaattcagttgcataagtacatgcaacaatatgccaaacttggagcaattgcttttagtaacgcccttactggagtggatatttcttaccaacaatactttgatattacatgtcgtttagctgaggcaatgaaaaactacggtatgaaaccggaaggacatattgctttgtgcagtgaaaattgtgaagaatttttcatccctgtgcttgctggtctttacattggagtaactgtcgcacctactaatgaaatttacacattgcgtgaacttaatcacagtttgggcatcgcacaaccaactattgtattcagctccagaaaaggcttacctaaagttttagaagtgcaaaaaacagttacatgcatcaaaacaattgttattttagatagtaaagtaaactttggaggctacgattgtgtggaaacttttattaagaaacatgtagaattaggttttccagcaactagctttgtacccattgatgtaaaggaccgtaaacatcacattgctttgcttatgaattcttctggctctactggtttacctaaaggtgtagagattacccacgaaggaacagttacaagattctcacacgctaaggatccaatttacggaaaccaagtttcacctggtactgctattttaactgtcgttccgttccatcatggatttggaatgtttaccactttaggatactttgcttgtggataccgtattgtaatgttaacaaaattcgatgaagaactatttttgagaactttgcaagattataagtgtaccagtgttattcttgtaccaacgttatttgctattctcaacaggagtgaattgctcgataagttcgatttatctaatctaactgaaattgcttctggtggagctcctttggcaaaagaaattggtgaagcagtcgctagaagatttaatctacccggtgtccgtcagggttacggattgacagaaacgacatctgcatttattattaccccagaaggtgatgataaacctggagcatctggaaaagtagtacccttattcaaagtaaaaattattgatcttgacactaaaaaaactttgggtgtcaaccgacgaggagagatctgtgtaaaaggtccgagtcttatgttaggctacacaaacaatccggaagcaacaagagaaactattgatgaagagggttggttgcacaccggagatattggatattacgacgaagacgaacatttcttcattgtagatcgtttgaaatcattaatcaaatacaaggggtaccaggtaccacctgctgaattggaatccgttcttttgcaacatccaaatatctttgatgctggtgtggctggtgtccccgattctgaagctggtgaacttccaggggctgtagttgtaatggaaaaaggaaaaactatgactgaaaaggaaattgtggattatgttaatagtcaagtagtgaaccacaaacgtctgcgtggtggcgttcgttttgtggatgaagtacctaaaggtctaactggtaaaattgatgctaaagtaattagagaaattcttaagaaaccacaagccgggggg

SEQ ID NO: 6 - protein, 548 aa, Luciola italica
METEREENVVYGPLPFYPIEEGSAGIQLHKYMQQYAKLGAIAFSNALTGVDISYQQYFDITCRLAEAMKNYGMKPEGHIALCSENCEEFFIPVLAGLYIGVTVAPTNEIYTLRELNHSLGIAQPTIVFSSRKGLPKVLEVQKTVTCIKTIVILDSKVNFGGYDCVETFIKKHVELGFPATSFVPIDVKDRKHHIALLMNSSGSTGLPKGVEITHEGTVTRFSHAKDPIYGNQVSPGTAILTVVPFHHGFGMFTTLGYFACGYRIVMLTKFDEELFLRTLQDYKCTSVILVPTLFAILNRSELLDKFDLSNLTEIASGGAPLAKEIGEAVARRFNLPGVRQGYGLTETTSAFIITPEGDDKPGASGKVVPLFKVKIIDLDTKKTLGVNRRGEICVKGPSLMLGYTNNPEATRETIDEEGWLHTGDIGYYDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 7 - DNA, 1641 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaggggtaccaggtaccacctgctgaattggaatccgttcttttgcaacatccaaatatctttgatgctggtgtggctggtgtccccgattctgaagctggtgaacttccaggggctgtagttgtaatggaaaaaggaaaaactatgactgaaaaggaaattgtggattatgttaatagtcaagtagtgaaccacaaacgtctgcgtggtggcgttcgttttgtggatgaagtacctaaaggtctaactggtaaaattgatgctaaagtaattagagaaattcttaagaaaccacaagccggggggtaa

SEQ ID NO: 8 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 9 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcggtattgttacaacaccccaacatcttcgacgcgggcgtggcaggtcttcccgacgatgacgccggtgaacttccgggcgccgtggttgttttggagaagggaaagacgatgacggaaaaagagatcgtggattacgtaaacagtcaagtaacaaccgcgaaaaagttgcgcggaggagttgtgtttgtggacgaagtaccgaaaggtcttaccggaaaactcgacgctagaaaaatcagagagatcctcataaaggcccagaagggcggaaagtccaaattg

SEQ ID NO: 10 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESVLLQHPNIFDAGVAGLPDDDAGELPGAVVVLEKGKTMTEKEIVDYVNSQVTTAKKLRGGVVFVDEVPKGLTGKLDARKIREILIKAQKGGKSKL

SEQ ID NO: 11 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatccaatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttggccaaaagtactctgattgacaaatacgatttatctaatttacacgaaattgcttctgggggcgcacctctttcgaaagaagtcggggaagcggttgcaaaacgcttccatcttccagggatacgacaaggatatgggctcactgagactactagcgctattctggtaacacccaagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcggtattgttacaacaccccaacatcttcgacgcgggcgtggcaggtcttcccgacgatgacgccggtgaacttccgggcgccgtggttgttttggagaagggaaagacgatgacggaaaaagagatcgtggattacgtaaacagtcaagtaacaaccgcgaaaaagttgcgcggaggagttgtgtttgtggacgaagtaccgaaaggtcttaccggaaaactcgacgctagaaaaatcagagagatcctcataaaggcccagaagggcggaaagtccaaattg

SEQ ID NO: 12 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESVLLQHPNIFDAGVAGLPDDDAGELPGAVVVLEKGKTMTEKEIVDYVNSQVTTAKKLRGGVVFVDEVPKGLTGKLDARKIREILIKAQKGGKSKL

SEQ ID NO: 13 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatccaatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaactgcgttactagtaccaaccctattttcattcttggccaaaagtactctgattgacaaatacgatttatctaatttacacgaaattgcttctgggggcgcacctctttcgaaagaagtcggggaagcggttgcaaaacgcttccatcttccagggatacgacaaggatatgggctcactgagactactagcgctattctggtaacacccaagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcggtattgttacaacaccccaacatcttcgacgcgggcgtggcaggtcttcccgacgatgacgccggtgaacttccgggcgccgtggttgttttggagaagggaaagacgatgacggaaaaagagatcgtggattacgtaaacagtcaagtaacaaccgcgaaaaagttgcgcggaggagttgtgtttgtggacgaagtaccgaaaggtcttaccggaaaactcgacgctagaaaaatcagagagatcctcataaaggcccagaagggcggaaagtccaaattg

SEQ ID NO: 14 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESVLLQHPNIFDAGVAGLPDDDAGELPGAVVVLEKGKTMTEKEIVDYVNSQVTTAKKLRGGVVFVDEVPKGLTGKLDARKIREILIKAQKGGKSKL

SEQ ID NO: 15 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatccaatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaactgcgttactagtaccaaccctattttcattcttggccaaaagtactctgattgacaaatacgatttatctaatttacacgaaattgcttctgggggcgcacctctttcgaaagaagtcggggaagcggttgcaaaaggcttccatcttccagggatacgccaaggatatgggctcactgagactactagtgctattctggtaacacccatcggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcggtattgttacaacaccccaacatcttcgacgcgggcgtggcaggtcttcccgacgatgacgccggtgaacttccgggcgccgtggttgttttggagaagggaaagacgatgacggaaaaagagatcgtggattacgtaaacagtcaagtaacaaccgcgaaaaagttgcgcggaggagttgtgtttgtggacgaagtaccgaaaggtcttaccggaaaactcgacgctagaaaaatcagagagatcctcataaaggcccagaagggcggaaagtccaaattg

SEQ ID NO: 16 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKGFHLPGIRQGYGLTETTSAILVTPIGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESVLLQHPNIFDAGVAGLPDDDAGELPGAVVVLEKGKTMTEKEIVDYVNSQVTTAKKLRGGVVFVDEVPKGLTGKLDARKIREILIKAQKGGKSKL

SEQ ID NO: 17 - DNA, 1644 bp, Artificial Sequence
  Description of Artificial Sequence Synthetic polynucleotide
atggaggacgccaagaacatcaagaagggaccagcccccttctaccccctggaggacggcacagccggcgagcagctgcacaaggccatgaagcggtacgccctggtgccaggcaccatcgccttcaccgacgcccacatcgaggtgaacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggagccctgttcatcggcgtggccgtggccccagccaacgacatctacaacgagcgggagctgctgaacagcatgaacatcagccagcccaccgtggtgttcgtgagcaagaagggcctgcagaagatcctgaatgtgcagaagaagctgcccatcatccagaagatcatcatcatggacagcaagaccgattaccagggcttccagagcatgtacaccttcgtgaccagccacctgcccccaggcttcaacgagtacgacttcgtgcccgagagcttcgaccgggacaagaccatcgccctgatcatgaacagcagcggcagcaccggcctgcccaagggcgtggccctgccccaccgggccctgtgcgtgcggttcagccacgccagagaccccatcttcggcaaccagatcgcccccgacaccgccatcctgagcgtggtgcccttccaccacggcttcggcatgttcaccaccctgggctacctgatctgcggcttccgggtggtgctgatgtacaggttcgaggaggagctgttcctgcggagcctgcaggactacaagatccagaccgccctgctggtgcccaccctgttcagcttcctggccaagagcaccctgatcgacaagtacgacctgagcaacctgcacgagatcgcctctggcggagccccactgagcaaggaggtgggcgaggccgtggccaagggcttccacctgccaggcatccggcagggctacggcctgaccgagaccaccagcgccatcctggtgacccccatcggcgacgacaagcccggagccgtgggcaaggtggtgcccttcttcgaggccaaggtggtggacctggacaccggcaagaccctgggcgtgaaccagagaggcgagctgtgcgtgagaggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtataaaggctatcaggtcgcccccgccgaactggaaagcgtcctgctgcagcaccctaacatcttcgatgccggagtggctggactgccagacgatgacgcaggagagctgcctggagctgtggtcgtgctggaaaagggcaaaactatgaccgagaaggaaatcgtcgattacgtgaacagccaggtgaccacagccaagaaactgcgaggaggagtcgtgttcgtcgacgaggtgcccaagggactgacaggcaaactggacgcacgcaagattagggagatcctgattaaggcacagaaggggggaaagatt

SEQ ID NO: 18 - protein, 548 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKGFHLPGIRQGYGLTETTSAILVTPIGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESVLLQHPNIFDAGVAGLPDDDAGELPGAVVVLEKGKTMTEKEIVDYVNSQVTTAKKLRGGVVFVDEVPKGLTGKLDARKIREILIKAQKGGKI

SEQ ID NO: 19 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatcctatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaactgcgttactagtaccaaccctattttcattcttggccaaaagtactctgattgacaaatacgatttatctaatttacacgaaattgcttctgggggcgcacctctttcgaaagaagtcggggaagcggttgcaaaaggcttccatcttccagggatacgccaaggatatgggctcactgagactactagtgctattctggtaacacccatcggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatccatattgttacaacaccccaacatccgggacgcgggcgtggcaggtcttcccgacgatgacgccggtgaacttcccgccgccgttgttgttttggagcacggaaagacgatgactgaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 20 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKGFHLPGIRQGYGLTETTSAILVTPIGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIRDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 21 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatccaatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttggccaaaagtactctgattgacaaatacgatttatctaatttacacgaaattgcttctgggggcgcacctctttcgaaagaagtcggggaagcggttgcaaaacgcttccatcttccagggatacgacaaggatatgggctcactgagactactagtgctattctgattacacccaagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 22 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 23 - DNA, 1650 bp, Artificial Sequence
  Description of Artificial Sequence Synthetic polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagagctctctgcgtcagattctcgcacgccagagatccaatatttggcaatcaaatcgctccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaactgcgttactagtaccaaccctattttcattcttggccaaaagtactctgattgacaaatacgatttatctaatttacacgaaattgcttctgggggcgcacctctttcgaaagaagtcggggaagcggttgcaaaaggcttccatcttccagggatacgccaaggatatgggctcactgagactactagtgctattctggtaacacccatcggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatccatattgttacaacaccccaacatccgggacgcgggcgtggcaggtcttcccgacgatgacgccggtgaacttcccgccgccgttgttgttttggagcacggaaagacgatgactgaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 24 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 25 - protein, 5 aa, Artificial Sequence
  Description of Artificial Sequence Synthetic peptide
GPLGS

SEQ ID NO: 26 - protein, 6 aa, Artificial Sequence
  Description of Artificial Sequence Synthetic 6xHis tag
HHHHHH

SEQ ID NO: 27 - protein, 12 aa, Artificial Sequence
  Description of Artificial Sequence Synthetic peptide
MRGSHHHHHHGS

SEQ ID NO: 28 - DNA, 36 bp, Artificial Sequence
  Description of Artificial Sequence Synthetic primer
agaaaccacaagccggggggtaaatcggtcaaaatg

SEQ ID NO: 29 - DNA, 43 bp, Artificial Sequence
  Description of Artificial Sequence Synthetic primer
gtagatcgtttgaaatcattaattaaatacaaggggtaccagg

SEQ ID NO: 30 - DNA, 50 bp, Artificial Sequence
  Description of Artificial Sequence Synthetic primer
gacgaagtaccgaaaggtcttaccggtaaactcgacgcaagaaaaatcag

SEQ ID NO: 31 - DNA, 46 bp, Artificial Sequence
  Description of Artificial Sequence Synthetic primer
gtggatgaagtacctaaaggtctaaccggtaaaattgatgctaaag

SEQ ID NO: 32 - DNA, 39 bp, Artificial Sequence
  Description of Artificial Sequence Synthetic primer
acgaggccctttcgtcttcacctggagaaatcataaaaa

SEQ ID NO: 33 - DNA, 39 bp, Artificial Sequence
  Description of Artificial Sequence Synthetic primer
cacggatccgcatgcgagctcgagaccccgggtcgacct

SEQ ID NO: 34 - DNA, 44 bp, Artificial Sequence
  Description of Artificial Sequence Synthetic primer
gccagagatccaatatttggcaatcaaatcgctccggatactgc

SEQ ID NO: 35 - DNA, 42 bp, Artificial Sequence
  Description of Artificial Sequence Synthetic primer
cttcaggattacaaaattcaaactgcgttactagtaccaacc

SEQ ID NO: 36 - DNA, 45 bp, Artificial Sequence
  Description of Artificial Sequence Synthetic primer
gggctcactgagactactagcgctattctggtaacacccaagggg

SEQ ID NO: 37 - DNA, 46 bp, Artificial Sequence
  Description of Artificial Sequence Synthetic primer
gagactactagtgctattctggtaacacccatcggggatgataaac

SEQ ID NO: 38 - DNA, 45 bp, Artificial Sequence
  Description of Artificial Sequence Synthetic primer
ctgagactactagtgctattctgattacacccaagggggatgata

SEQ ID NO: 39 - DNA, 49 bp, Artificial Sequence
  Description of Artificial Sequence Synthetic primer
gaacacttcttcatagttggccgcttgaagtctttgattaaatacaaag

SEQ ID NO: 40 - DNA, 54 bp, Artificial Sequence
  Description of Artificial Sequence Synthetic primer
aaaggatatcaggtggcccccgctgaattggaatcggtattgttacaacacccc

SEQ ID NO: 41 - DNA, 30 bp, Artificial Sequence
  Description of Artificial Sequence Synthetic primer
catccaaatatgcgtgatgctggtgtggct

SEQ ID NO: 42 - DNA, 54 bp, Artificial Sequence
  Description of Artificial Sequence Synthetic primer
gccggtgaacttccgggcgccgttgttgttatggagaagggaaagacgatgacg

SEQ ID NO: 43 - DNA, 39 bp, Artificial Sequence
  Description of Artificial Sequence Synthetic primer
cttccgggcgccgtggttgttttggagaagggaaagacg

SEQ ID NO: 44 - DNA, 51 bp, Artificial Sequence
  Description of Artificial Sequence Synthetic primer
ccgaaaggtctcaccggaaaaatcgacgcaagagtaatcagagagatcctc

SEQ ID NO: 45 - DNA, 48 bp, Artificial Sequence
  Description of Artificial Sequence Synthetic primer
gagatcgtggattacgtaaacagtcaagtaacaaccgcgaaaaagttg

SEQ ID NO: 46 - DNA, 53 bp, Artificial Sequence
  Description of Artificial Sequence Synthetic primer
aaaatcgacgctagagtaatcagagagatcctcaaaaagccaaagaagggcgg

SEQ ID NO: 47 - DNA, 53 bp, Artificial Sequence
  Description of Artificial Sequence Synthetic primer
aaactcgacgctagaaaaatcagagagatcctcataaaggcccagaagggcgg

SEQ ID NO: 48 - DNA, 53 bp, Artificial Sequence
  Description of Artificial Sequence Synthetic primer
aaaatcgacgcaagagtaatcagagagatcctcaaaaagccacagaagggcgg

SEQ ID NO: 49 - DNA, 39 bp, Artificial Sequence
  Description of Artificial Sequence Synthetic primer
cggctgaagagcttaattaaatacaagggctaccaggtg

SEQ ID NO: 50 - DNA, 42 bp, Artificial Sequence
  Description of Artificial Sequence Synthetic primer
gtggaccggctgaagagcctgatcaagtataaaggctatcag

SEQ ID NO: 51 - protein, 546 aa, Artificial Sequence
  Description of Artificial Sequence Synthetic consensus polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKLDAKVIREILKKPQAKX

SEQ ID NO: 52 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVXPAELESXLLQHPNIFDAGVAGXPDXXAGELPXAVVVXEXGKTMTEKEIVDYVXSQVXXXKXLRGGVXFVDEVPKGLTGKLDARKIREILIKAKKGGKSKL

SEQ ID NO: 53 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVXPAELESXLLQHPNIFDAGVAGXPDXXAGELPXAVVVXEXGKTMTEKEIVDYVXSQVXXXKXLRGGVXFVDEVPKGLTGKXDARXIREILXKXXKGGKSKL

SEQ ID NO: 54 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESXLLQHPNIFDAGVAGLPDDDAGELPXAVVVLEXGKTMTEKEIVDYVXSQVTTAKKLRGGVVFVDEVPKGLTGKLDARKIREILIKAXKGGKSKL

SEQ ID NO: 55 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIRDAGVAGVPDSEAGELPGAVVVMEKGKTMTEKEIVDYVNSQVVNHKRLRGGVRFVDEVPKGLTGKIDAKVIREILKKPQAGG

SEQ ID NO: 56 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIAPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 57 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 58 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIAPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 59 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIRDAGVAGVPDSEAGELPGAVVVMEKGKTMTEKEIVDYVNSQVVNHKRLRGGVRFVDEVPKGLTGKIDAKVIREILKKPQAGG

SEQ ID NO: 60 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 61 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIRDAGVAGVPDSEAGELPGAVVVMEKGKTMTEKEIVDYVNSQVVNHKRLRGGVRFVDEVPKGLTGKIDAKVIREILKKPQAGG

SEQ ID NO: 62 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 63 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIAPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILVTPIGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAGG

SEQ ID NO: 64 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIRDAGVAGVPDSEAGELPGAVVVMEKGKTMTEKEIVDYVNSQVVNHKRLRGGVRFVDEVPKGLTGKIDAKVIREILKKPQAGG

SEQ ID NO: 65 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIAPDTAILSVVPFHHGFGMFTTLGYLICGFRVVLMYRFEEELFLRSLQDYKIQTALLVPTLFSFFAKSTLIDKYDLSNLHEIASGGAPLSKEVGEAVAKRFHLPGIRQGYGLTETTSAILVTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIRDAGVAGVPDSEAGELPGAVVVMEKGKTMTEKEIVDYVNSQVVNHKRLRGGVRFVDEVPKGLTGKIDAKVIREILKKPQAGG

SEQ ID NO: 66 - protein, 546 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRXXCVRFSHARDPIFGNQIXPDTAILSVVPFHHGFGMFTTLGYLICGFRVVLMYRFEEELFLRSLQDYKIQXALLVPTLFSFXAKSTLIDKYDLSNLHEIASGGAPLSKEVGEAVAKRFHLPGIRQGYGLTETTSAILXTPXGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVPPAELESVLLQHPNIXDAGVAGVPDSEAGELPGAVVVMEKGKTMTEKEIVDYVNSQVVNHKRLRGGVRFVDEVPKGLTGKIDAKVIREILKKPQAGG

SEQ ID NO: 67 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVIPFHHAFGMSTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 68 - protein, 550 aa, Artificial Sequence; identical to SEQ ID NO: 24
  Description of Artificial Sequence Synthetic polypeptide

SEQ ID NO: 69 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRXXCVRFSHARDPIFGNQIXPDTAILSVXPFHHXFGMXTTLGYLICGFRVVLMYRFEEELFLRSLQDYKIQXALLVPTLFSFXAKSTLIDKYDLSNLHEIASGGAPLSKEVGEAVAKRFHLPGIRQGYGLTETTSAILXTPXGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 70 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILXTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESXLLQHPNIFDAGVAGLPDDDAGELPXAVVVLEXGKTMTEKEIVDYVXSQVTTAKKLRGGVVFVDEVPKGLTGKLDARKIREILIKAXKGGKSKL

SEQ ID NO: 71 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKRFHLPGIRQGYGLTETTSAILXTPKGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESXLLQHPNIFDAGVAGLPDDDAGELPXAVVVLEXGKTMTEKEIVDYVXSQVTTAKKLRGGVVFVDEVPKGLTGKLDARKIREILIKAXKGGKSKL

SEQ ID NO: 72 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKGFHLPGIRQGYGLTETTSAILVTPIGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESXLLQHPNIXDAGVAGLPDDDAGELPXAVVVLEXGKTMTEKEIVDYVXSQVTTAKKLRGGVVFVDEVPKGLTGKLDARKIREILIKAXKGGKSKL